(19) United States
(12) Patent Application Publication
(10) Pub. No.: US 2015/0190501 A1
(43) Pub. Date: Jul. 9, 2015

(54) METHODS AND COMPOSITIONS FOR RAISING AN IMMUNE RESPONSE TO HIV

(71) Applicants: Jonathan Norden Weber, London (GB); Robin John Shattock, London (GB); Paul Francis James Mckay, London (GB); Sheena Mary Geraldine Mccormack, London (GB)

(72) Inventors: Jonathan Norden Weber, London (GB); Robin John Shattock, London (GB); Paul Francis James Mckay, London (GB); Sheena Mary Geraldine Mccormack, London (GB)

(73) Assignee: IMPERIAL INNOVATIONS LIMITED, London (GB)

(21) Appl. No.: 14/344,088

(22) PCT Filed: Sep. 12, 2012
(86) PCT No.: PCT/GB2012/052261

§ 371 Date: Jun. 26, 2014

Related U.S. Application Data
(60) Provisional application No. 61/533,619, filed on Sep. 12, 2011.
(60) Provisional application No. 61/533,635, filed on Sep. 12, 2011.

(51) Int. Cl. A61K 39/21; C12N 7/00

(52) U.S. Cl. CPC A61K 39/21; C12N 7/00; A61K 2039/53

(57) ABSTRACT

A method of raising an immune response in an individual against human immunodeficiency virus (HIV) the method comprising: (a) administering to the individual DNA or SFV encoding an HIV Env protein and optionally nucleic acid encoding one, any two or all three of the HIV Gag, Pol and Nef proteins; (b) subsequently administering to the individual viral vector encoding an HIV Env protein and optionally viral vector encoding one, any two or all three of the HIV Gag, Pol and Nef proteins; and (c) administering to the individual oligomeric HIV Env protein; and an adjuvant.

The present invention relates to methods and compositions for raising an immune response in an individual to human immunodeficiency virus (HIV).

The listing or discussion of an apparently prior-published document in this specification should not necessarily be taken as an acknowledgment that the document is part of the state of the art or is common general knowledge.

20th century vaccine history taught us that viral epidemics are best controlled through the widespread use of safe and specific vaccines (1). Although combination antiretroviral therapy (ART) has transformed life expectancy, and global initiatives such as PEPFAR and the Global Fund to fight AIDS, TB and Malaria have made these drugs widely available, treatment has had limited impact on the number of new infections. Whilst we hope that the strategic use of ART to prevent infection will increase, the need for a safe and effective preventive HIV vaccine remains one of the highest priorities for international public health, but progress is slow. After 25 disappointing years, including four negative efficacy trials, the recent phase III trial Sanofi-PasteurNaxGen ALVAC-rGP120 (RV144) “Thai” trial demonstrated significant protection against HIV infection through a prime-boost strategy with a canary-pox viral vector (ALVAC), followed by an ENV (GP120) protein boost (2). There were previously no correlates of protection reported, but it has been assumed that both broadly specific and durable cellular and humoral immune responses will be required, both of which have been shown to contribute towards protection during natural infections and in animal models. More recently, it has been reported from the RV144 trial that IgA binding to envelope is associated with increasing risk of infection whilst IgG binding to p70V1V2 was associated with reduced risk. See, for example, http://www.eurekalert.org/pub_releases/2012-04/dumc-fst040212.php. Whilst lymphoproliferative responses were seen in 89-90% of vaccinees assessed to ENV peptides (n=142) and 35% to peptides derived from p24, only 17% demonstrated γ-IFN Elispot responses to any HIV antigen. Cytotoxic T-cell responses were not assessed, neutralising antibodies (Nab) were negligible but GP120 binding antibodies to either gp120 (B/E) were seen in 99% of those assessed, leading to speculation that non-neutralising GP120 binding antibodies were protective, perhaps via antibody-dependent cell-mediated cytotoxicity (ADCC).

The failure of the HIV ENV GP120 trials (3, 4) lead to the development of T-cell-inducing vaccines based on genetically modified viral vectors (adenovirus, pox virus) which induce variable T-cell immunogenicity (5). Unfortunately, a phase III trial of MRK Ad5 HIV-1 gag-pol-nef was stopped prematurely due to futility and the possibility that the Adeno-5 vector increased susceptibility to infection could not be excluded (6, 7).

The EuroVacc EV02 trial compared the immune responses of healthy volunteers randomised to receive 2×4 mg priming DNA 4 weeks apart before 2×NYVAC-C to a group with no DNA priming receiving only 2×NYVAC-C (8, 9). EV03 showed that an additional priming injection with DNA-C broadened the specificity of the T-cell response and increased its magnitude, as well as confirming the immunogenicity of the combination in a larger and more diverse population (10). Multiclade vaccine candidates are being developed (11). Trimeric rGP140 is considered to be more immunogenic than the homologous monomer (GP120) (13-15). See also Wijesundara et al (2011) Immunol. Cell Biology 89, 367-374 for a review of HIV vaccination strategies.

A successful and safe HIV vaccine would be of global benefit but would also have considerable application across the general populations and high risk groups in well resourced countries. It has been suggested that an effective vaccine has the potential to prevent over 70 million infections over the course of 15 years. There would be strong political support for the purchase of such a vaccine in less well resourced settings.

A first aspect of the invention provides a method of raising an immune response in an individual against human immunodeficiency virus (HIV) the method comprising:

(a) administering to the individual DNA or Semliki Forest virus vector (SFV) encoding an HIV Env protein and optionally nucleic acid encoding one, any two or all three of the HIV Gag, Pol and Nef proteins;
(b) subsequently administering to the individual viral vector encoding an HIV Env protein and optionally viral vector encoding one, any two or all three of the HIV Gag, Pol and Nef proteins; and
(c) administering to the individual oligomeric HIV Env protein combined with an adjuvant.

Step (a) may be considered to be a priming step and step (b) and step (c) boosting steps. The immune response may be a humoral immune response, but typically includes both a humoral and cell-mediated immune response.

Preferably the individual is a human individual. Typically, the human is a person who is at risk of HIV infection, such as a sex worker, a man who has sex with a man or an injector of drugs, although in some countries the general population is at risk. In any event, it may be useful to use the method in relation to the general population as is the case with other strategies for preventing viral infection. The method may also be considered a method of vaccinating against HIV infection, or a method of preventing HIV infection in an individual. It may also be considered to be a method of raising antibodies against HIV. It may also be considered to be a method of raising a T-cell response to HIV.

Preferably the HIV is HIV-1. Typically the HIV Env protein sequence, and the HIV Gag, Pol and Nef protein sequences are selected as being from an HIV clade appropriate to the individual. HIV variants are divided into three groups: M, for major, N, and O, for other or outlier. Within the M-group there are at least ten subtypes or clades: A, B, C, D, E, F, G, H, I, J, and K. The B-clade is dominant in US, Europe, Southeast Asia, and South America. Clades E and C are dominant in Asia and A, C, and D are dominant in Africa. Each of the five clades differs from each other by as much as 35%.

| Clade | Occurrence Location |
|---|---|
| B | US, Europe, South America, |
| | Southeast Asia, Australia |
| C | Asia, Africa, India |
| D | Africa |
| E | Asia |

It is preferred, for example, that if the individual resides in Africa, the Env, Gag, Pol and Nef protein sequences used are from clade C, whereas if the individual resides in Europe or the USA, the Env, Gag, Pol and Nef protein sequences used are from clade B. Suitable sequences of the Env, Gag, Pol and Nef proteins are known, for example from the HIV sequence database (http://hiv-web.lanl.gov, incorporated herein by reference) (see also The 2011 Nucleic Acids Research Database Issue and the online Molecular Biology Database Collection, Galperin & Cochrane (2011) Nucl. Acies Res. 39, D1-D6, incorporated herein by reference. US Patent Application Publication No US2006/0275897, incorporated herein by reference, includes details of plasmid vectors that encode HIV Env, Gag, Pol and Nef proteins.

It is particularly preferred that the Env, and if present, the Gag, Pol, and Nef proteins are from the same HIV clade, and preferably the same HIV variant. It is also particularly preferred if in each of steps (a), (b) and (c), the DNA and viral vector encode HIV proteins from the same, single clade (and preferably the same, single HIV variant), and that the Env protein is also from the same, single clade. Thus, it is particularly preferred if the method makes use of components from a single HIV clade such as clade B or clade C or clade D or clade E.

In step (a) the DNA may be naked DNA, such as one or more plasmid DNAs or PCR products, or the DNA may be comprised in one or more adenoviral vectors. By “naked DNA” we include the meaning that the DNA is substantially free of non-DNA components, such as proteins and lipids. In particular, naked DNA does not include viral coat proteins and it is not packaged in a viral particle or present in a liposome or other vehicle that coats or encapsulates the DNA. Plasmid DNA is preferred naked DNA.

Suitable adenoviral vectors, or adeno-associated viral vectors are known in the art, such as those described in Lin et al (2009) J. Virol. 83, 12738-12750, and Rollier et al (2011) Current Opinion in Immunology 23, 377-382, both of which are incorporated herein by reference. It is preferred that the adenovirus or adeno-associated virus on which the vector is based is not one that is found in the individual. For example, it is preferred that the adenoviral vector of adeno-associated viral vector is a simian vector (SAd), such as a chimpanzee vector (eg an AdC based vector), which are not generally found in humans. Human adenovirus or adeno-associated virus vectors may be used, and in that case it is preferred if they are from a rare human serotype, such as AdHu35, AdHu28 or modification of the AdHu5 capsid. A preferred vector is a SAd which encodes the Env protein, and optionally also the Gag Pol and Nef proteins. Suitable CN54 Gap Pol Nef and Env gene sequences are described in Reference 9, incorporated herein by reference. The adenovirus or adeno-associated virus vector is one that is suitable for human use, and can act as a delivery system for genes expressing the polypeptides.

Semliki Forest virus vectors are well known in the art, for example see Liljeström & Garoff (1991) A new generation of animal cell expression vectors based on the Semliki Forest virus replicon Biotechnology 9: 1356-1361; Piver et al (2005) Gene Therapy 12, S11-S117; and Quetglas et al (2011) Gene Therapy, 7 July Semliki Forest virus vector engineered to express IFNα induces efficient elimination of established tumors, each of which is incorporated herein by reference.

In step (a) DNA or SFV encoding one, any two or all three of the HIV Gag, Pol and Nef proteins, in addition to DNA or SFV encoding the HIV Env protein, may usefully be present and may be on the same DNA molecule or different DNA molecules. Thus, in step (a) DNA or SFV encoding Env alone or Env+Gag or Env+Pol or Env+Nef or Env+Gag+Pol or Env+Gag+Nef and so one may be used. Typically in step (a) nucleic acid encoding each of HIV Env, Gag, Pol and Nef proteins is administered to the individual. In step (a) one, two, three or four separate DNA molecules may be used, typically administered simultaneously, but it is preferred if only one or two are used. In a preferred embodiment, the DNA or SFV encoding Env is present on one DNA molecule (such as a plasmid), and DNA or SFV encoding Gag, Pol and Nef is present on another DNA molecule (such as a plasmid). It may be useful to include in the one or more of the DNA molecules, particularly naked DNA molecules, an enhancer and/or promoter such as the CMV enhancer/promoter and/or the HTLV-1R promoter which has been shown to enhance the immunogenicity of DNA vaccines encoding gag, pot, nef and env in mice and cynomologous monkeys (Reference 16, incorporated herein by reference).

Typically, the coding region for the one or more of the HIV proteins is codon optimised so that the codons used are appropriate for expression in the individual, for example the codons may be optimised for expression of the Env, Gag, Pol and/or Nef polypeptides in a human cell. Codon optimisation of the genes encoding Env, Gag, Pol and Nef is described in Gao et al (2003) AIDS Res. Human Retroviruses 19, 817-823, incorporated herein by reference. Codon optimisation may be used in the DNA administered in step (a) or in the viral vector in step (b) or both. Different codon optimisation may be used in the coding regions of the DNA in step (a) and the viral vector of step (b).

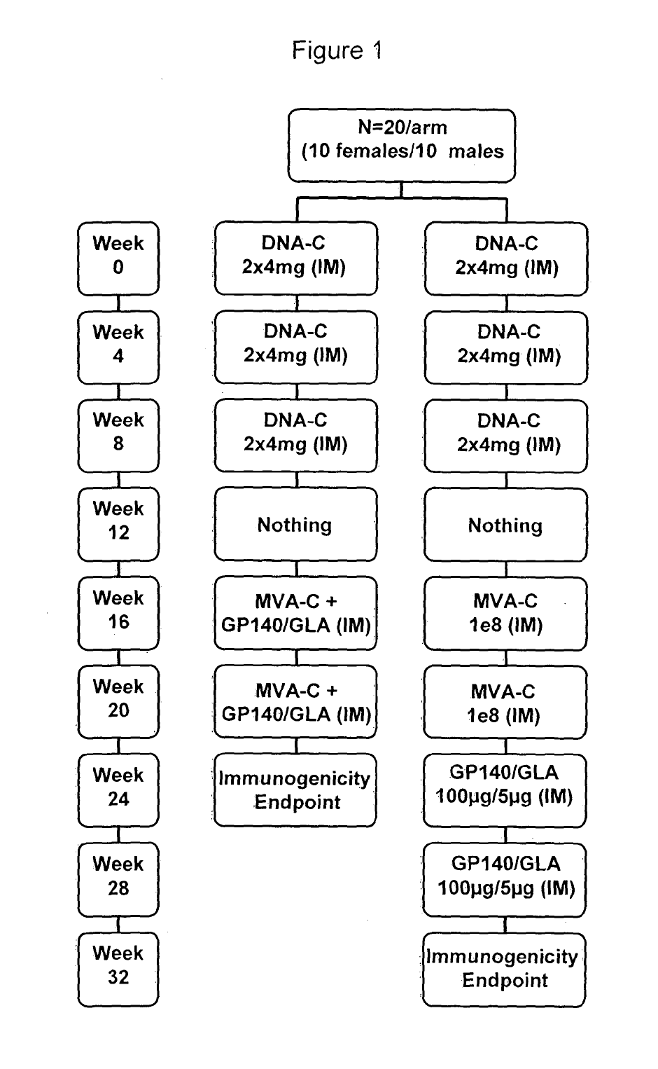

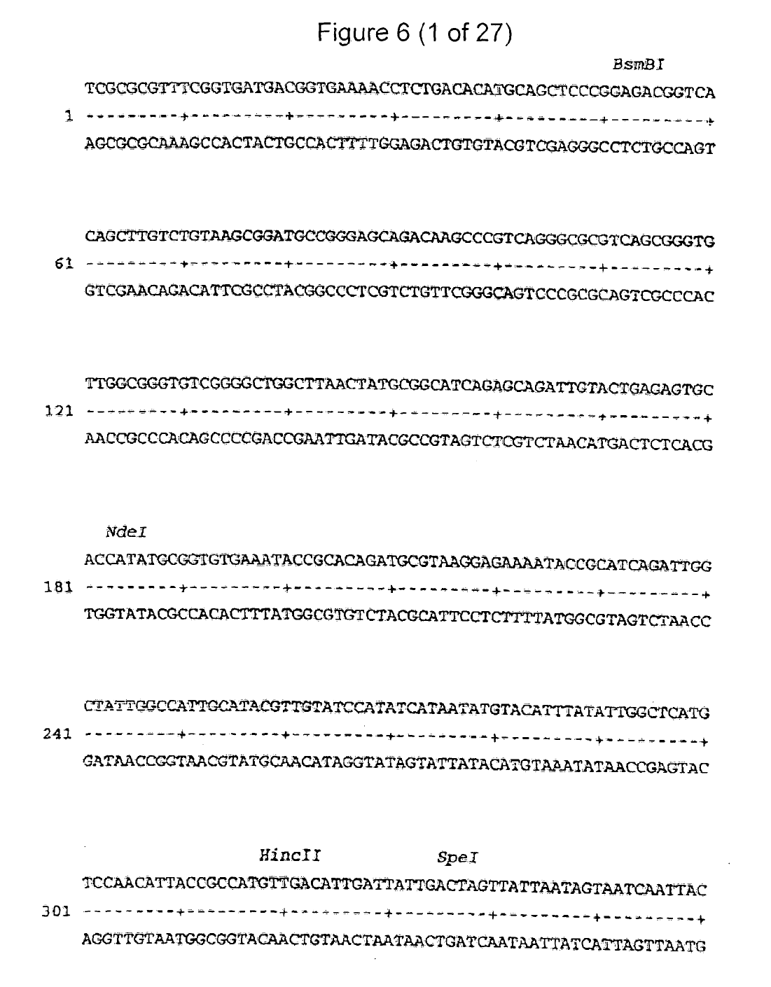

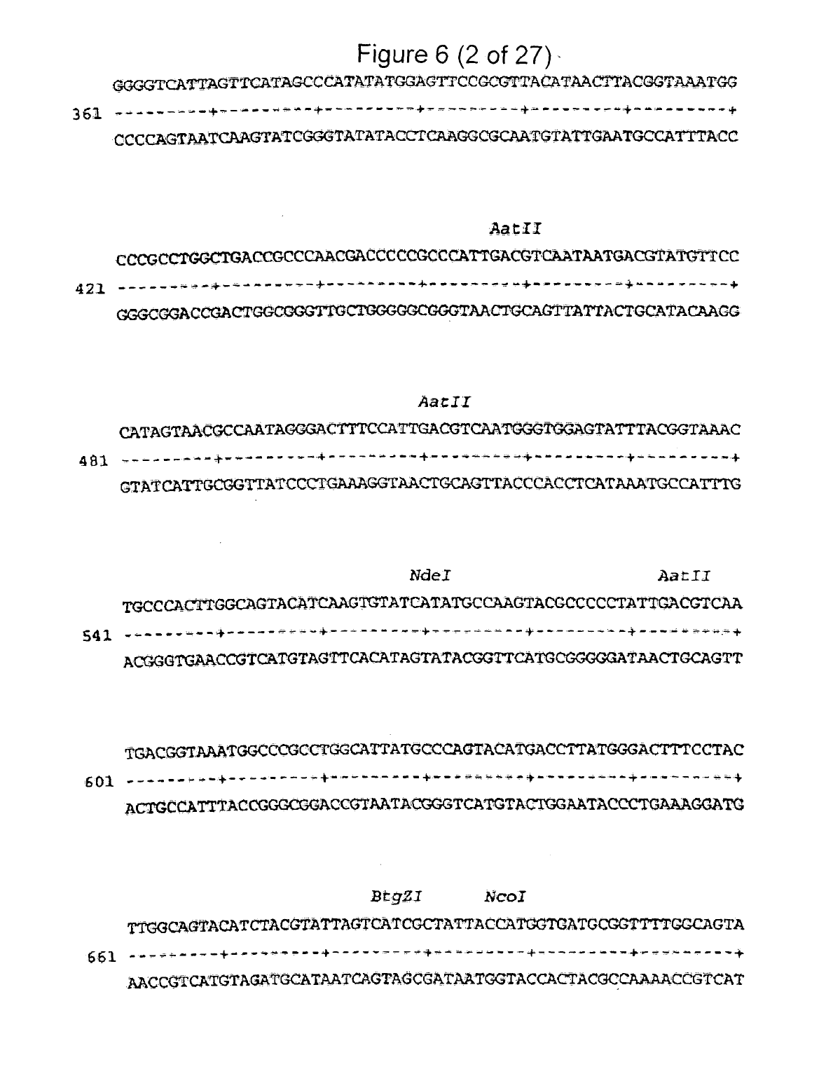

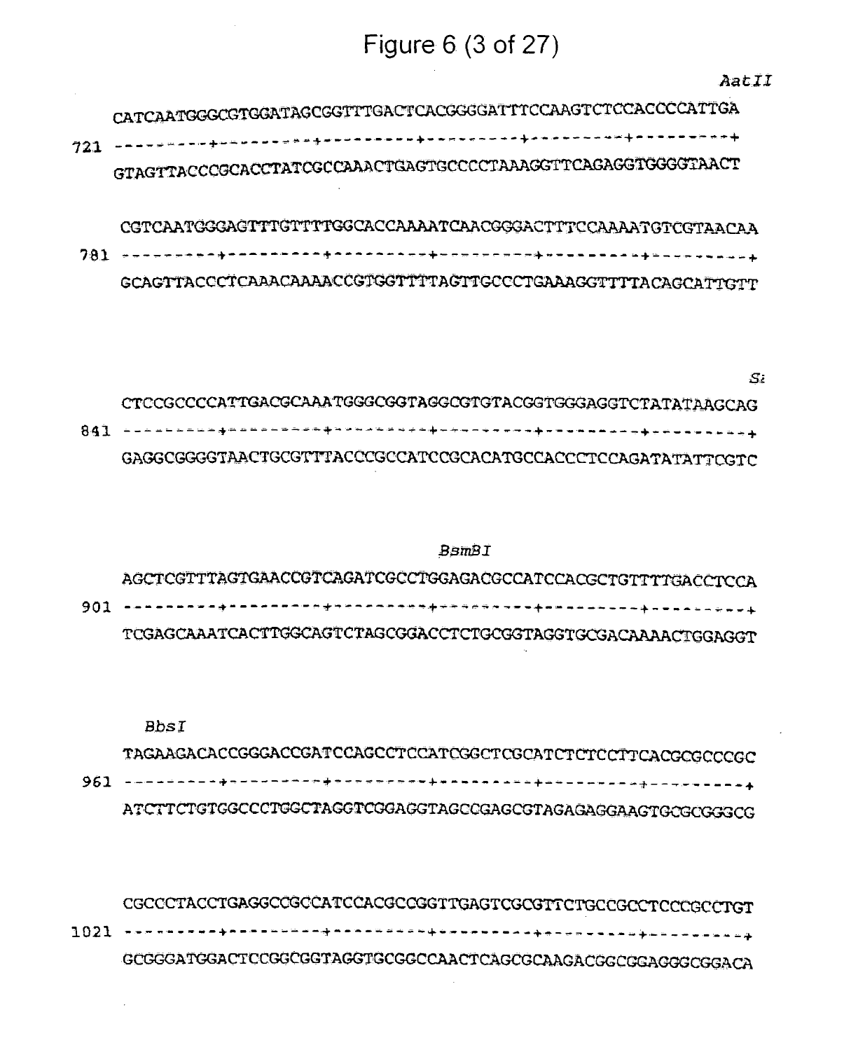

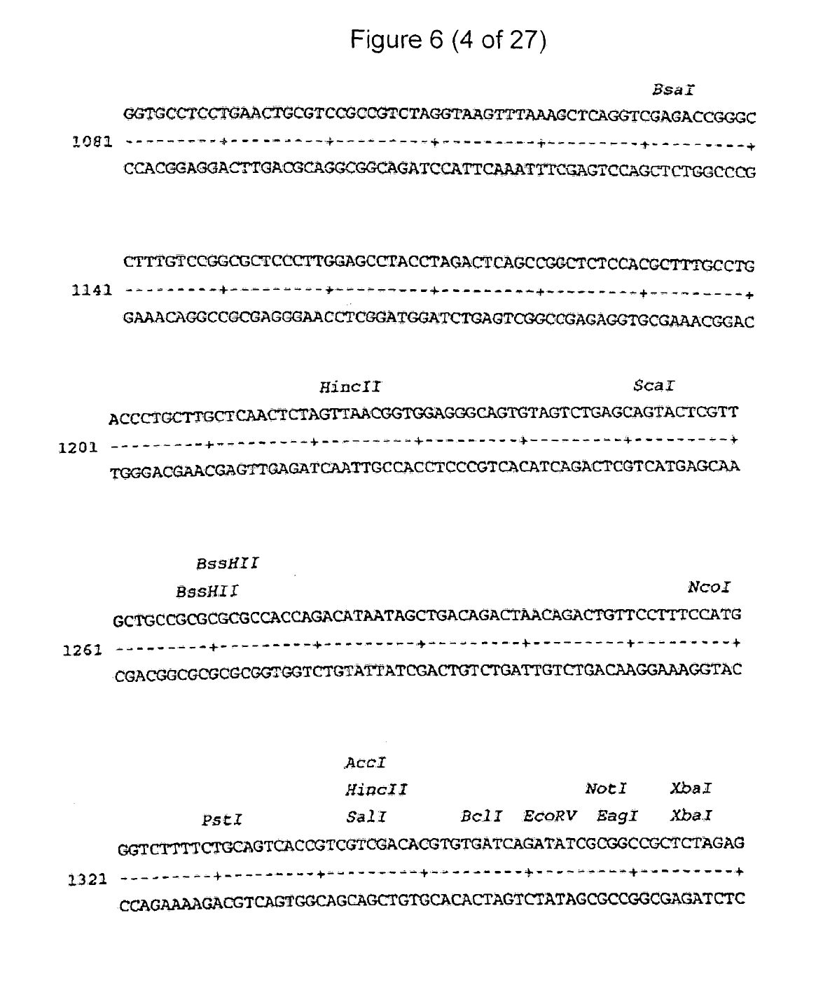

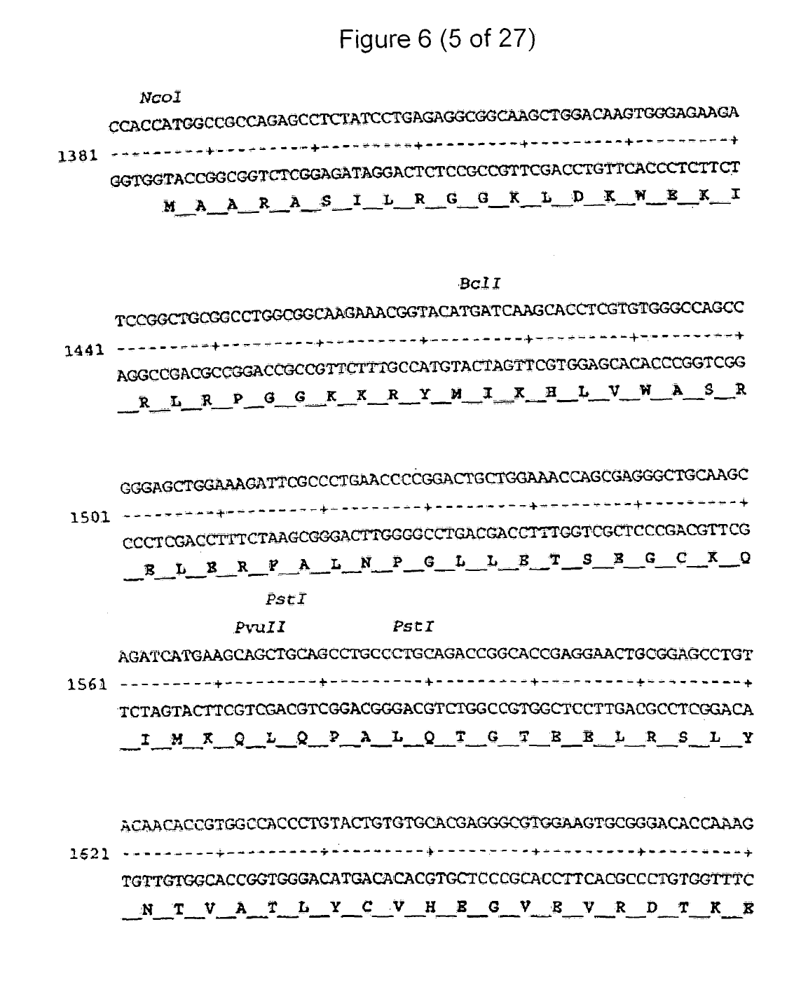

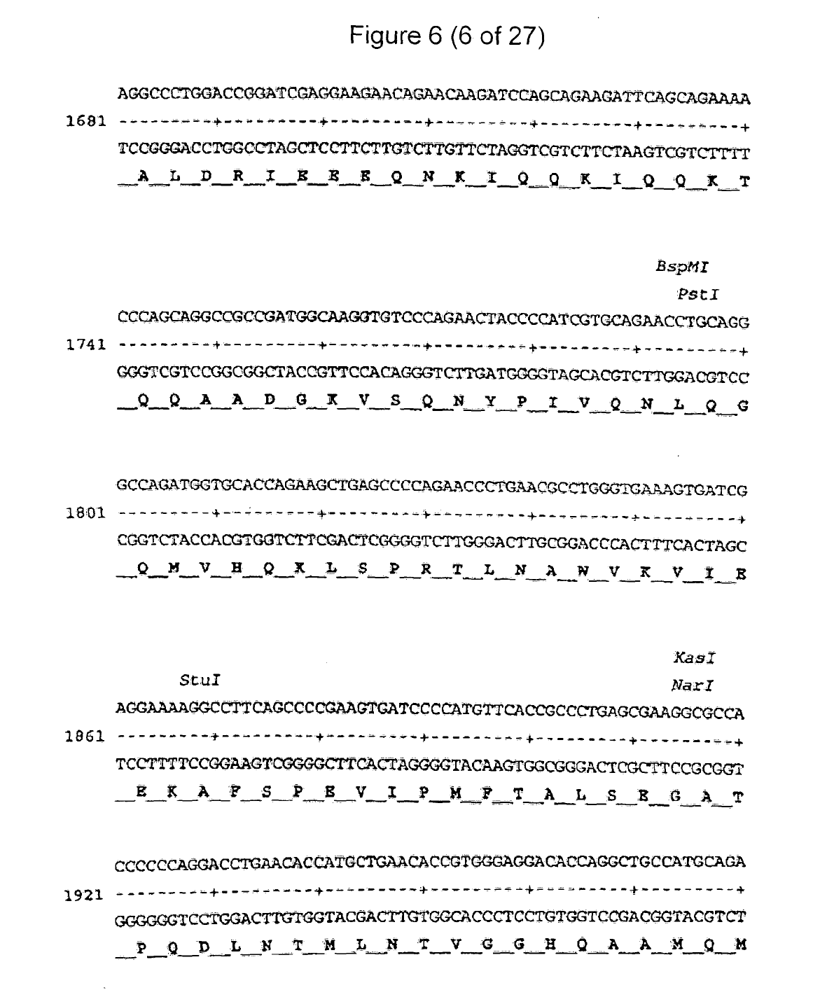

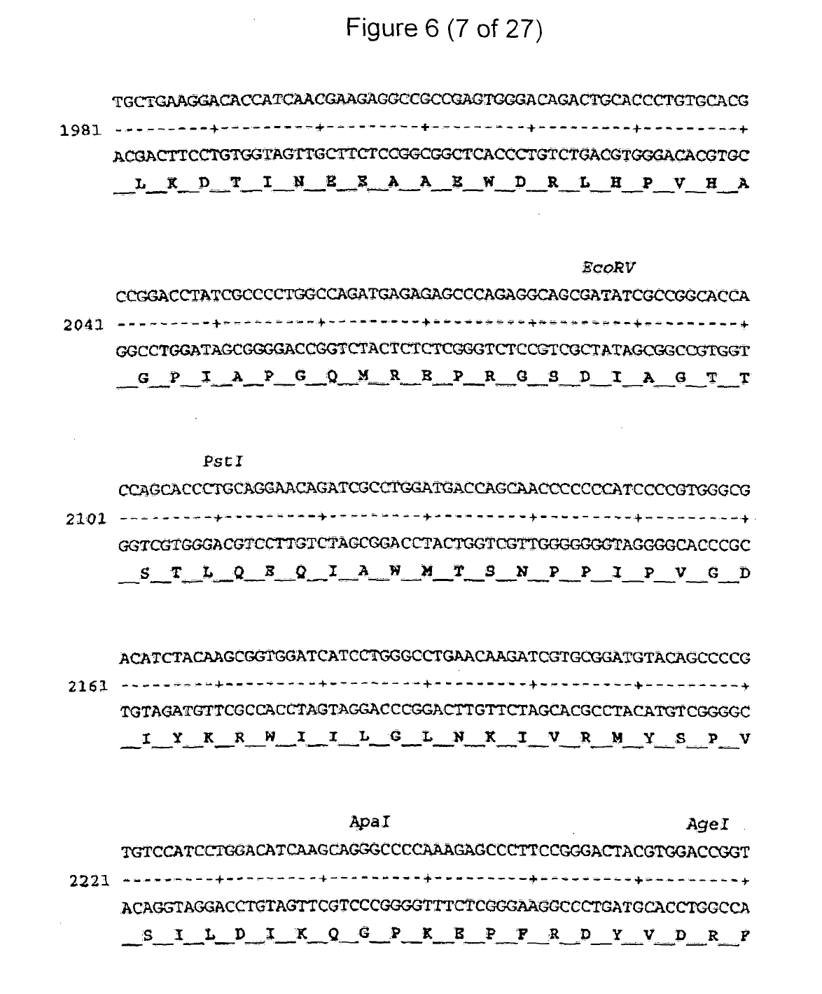

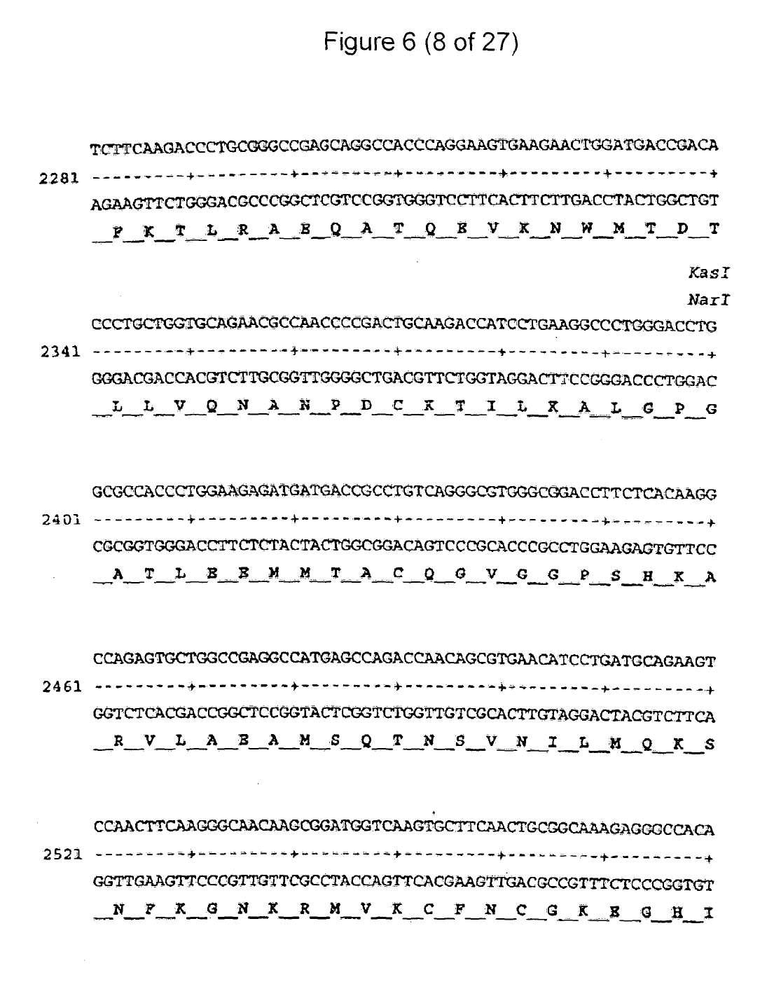

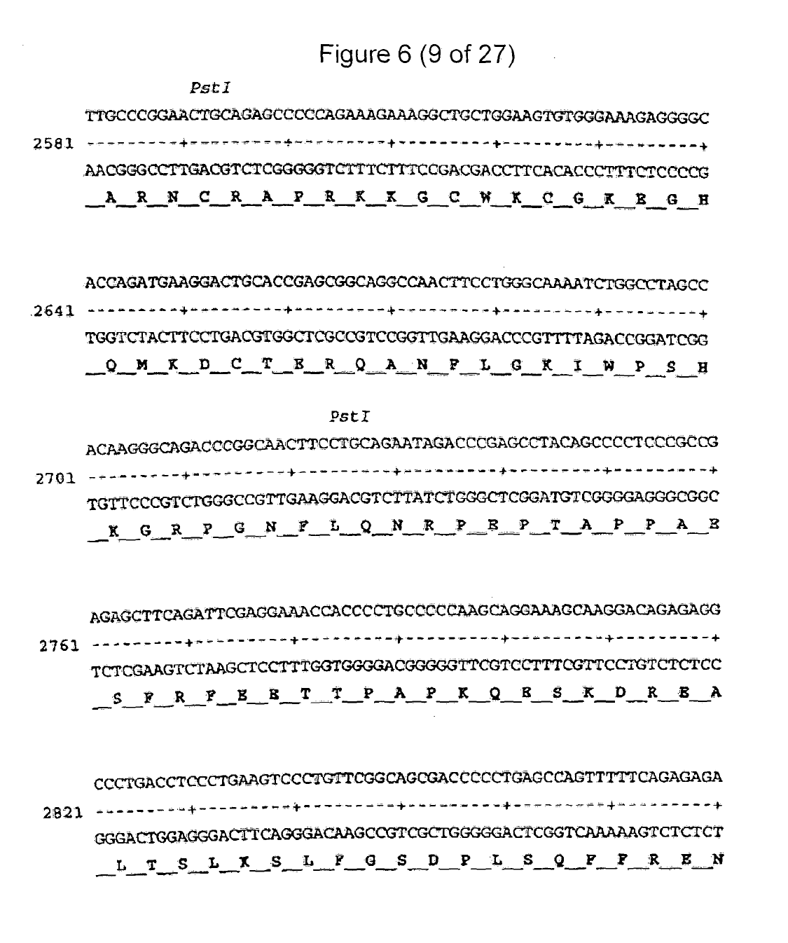

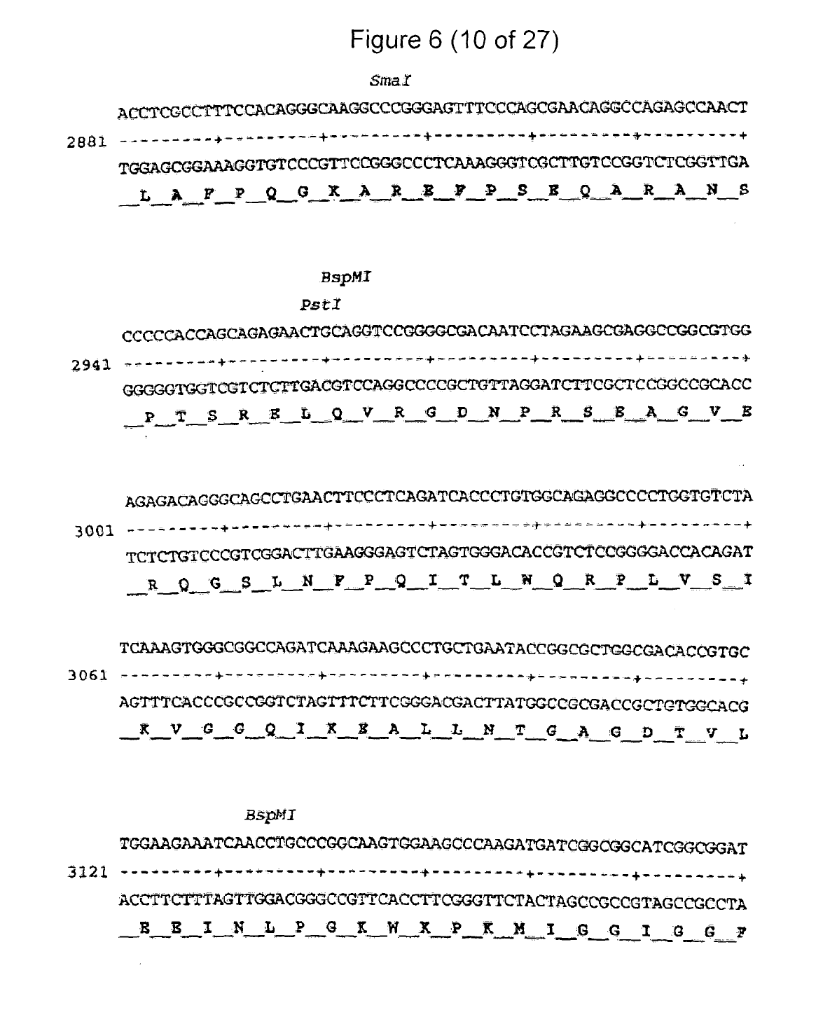

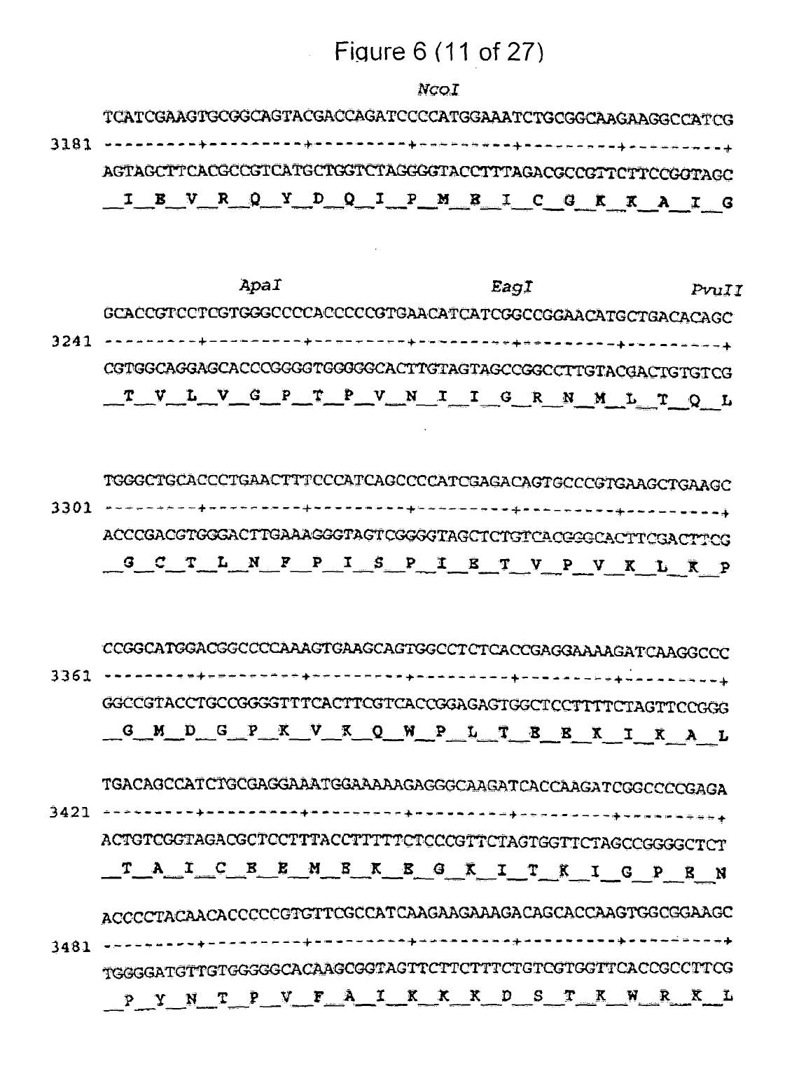

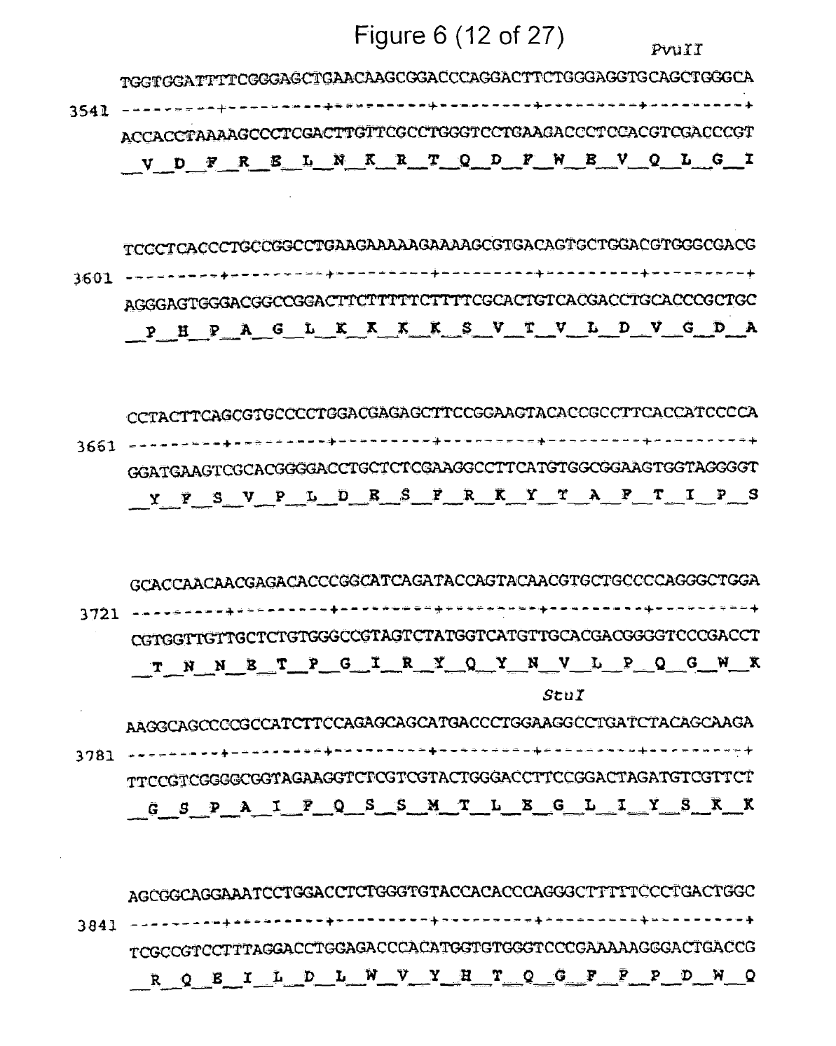

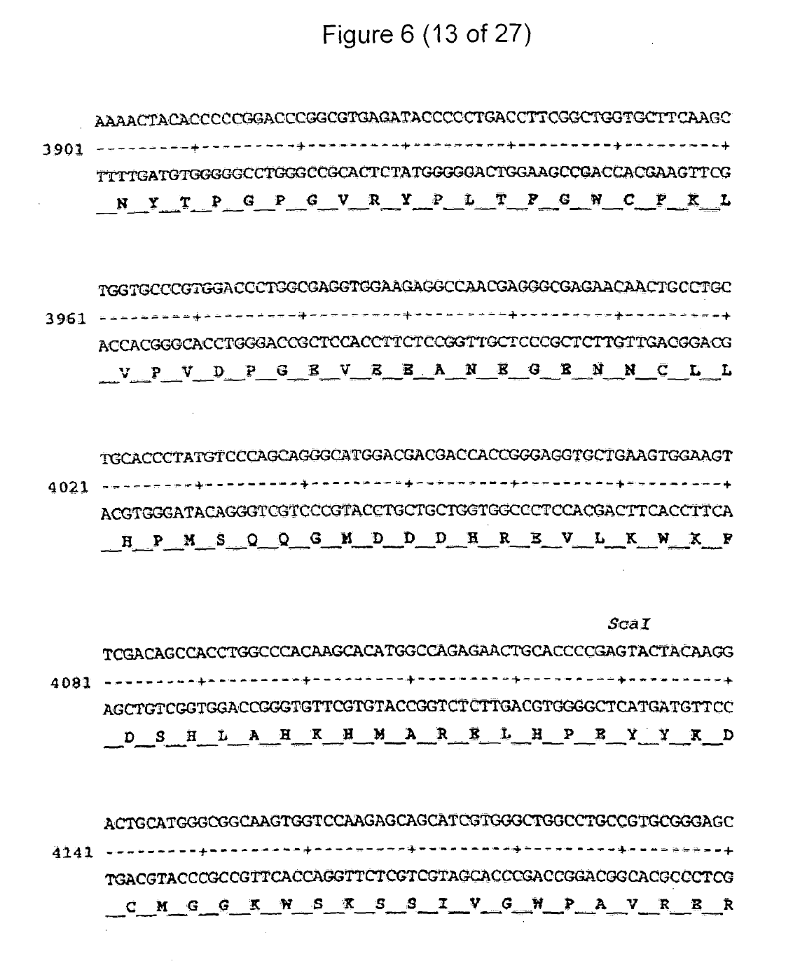

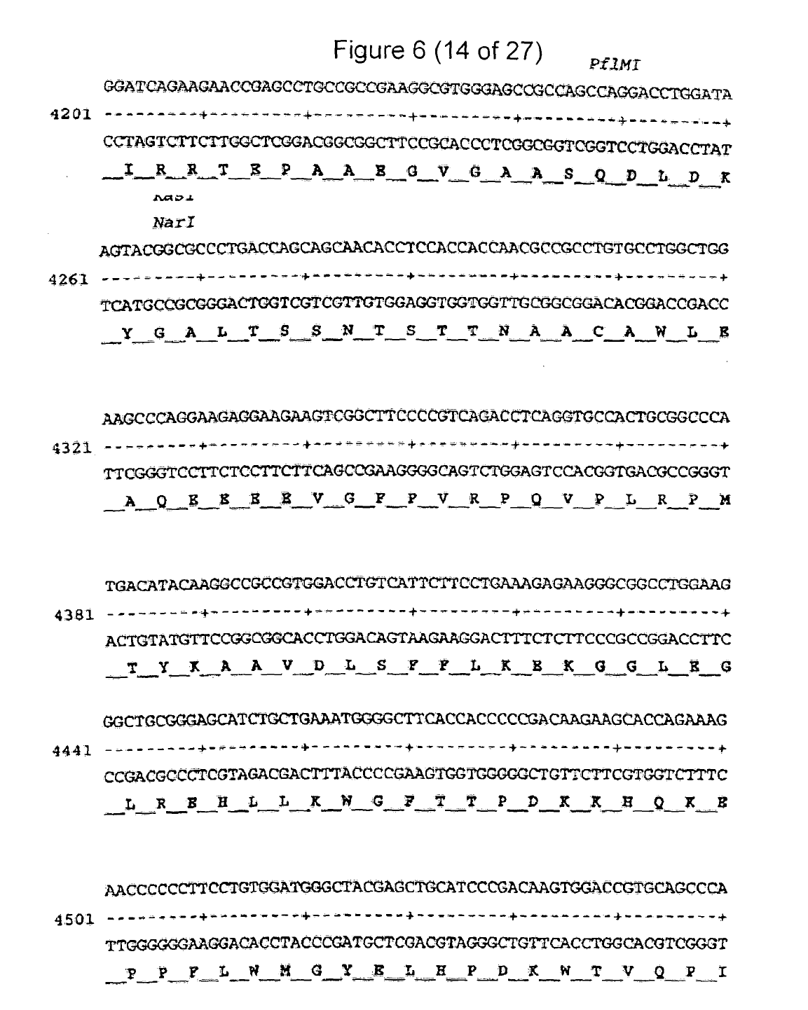

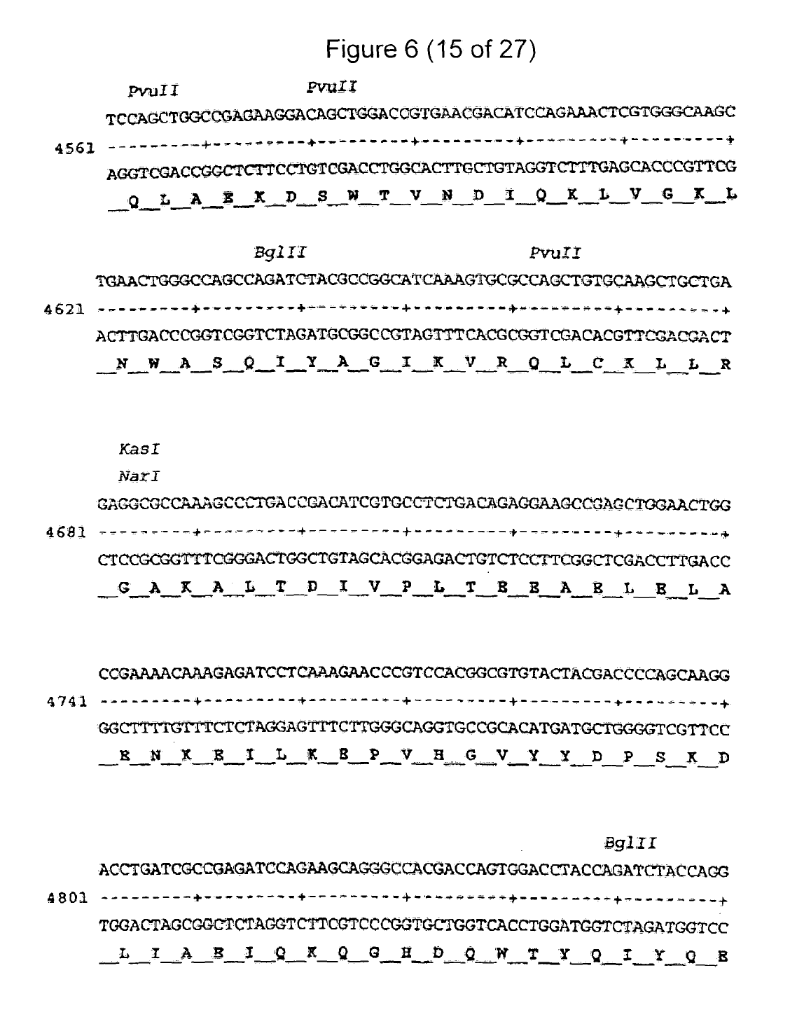

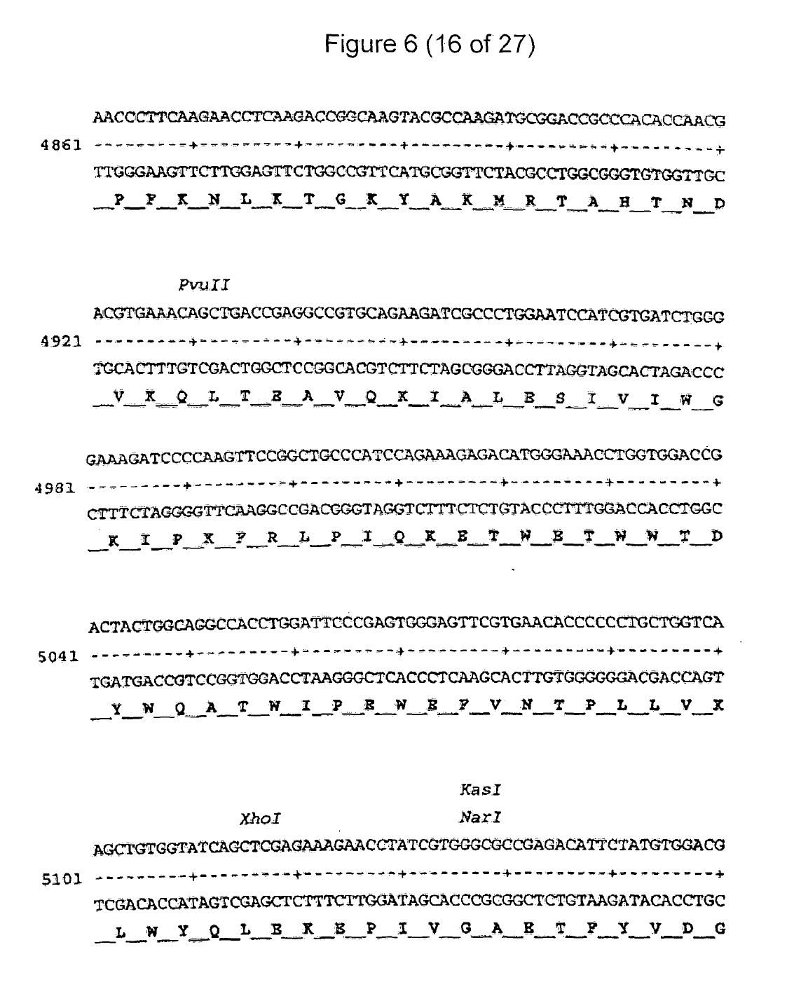

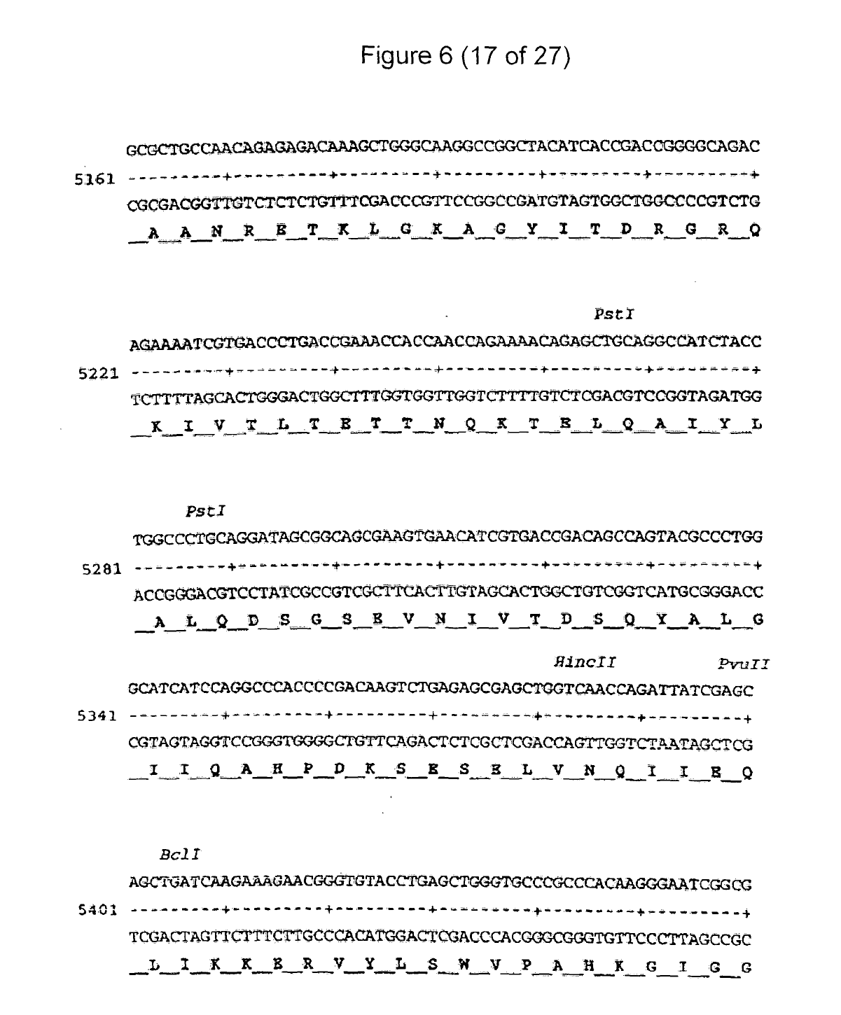

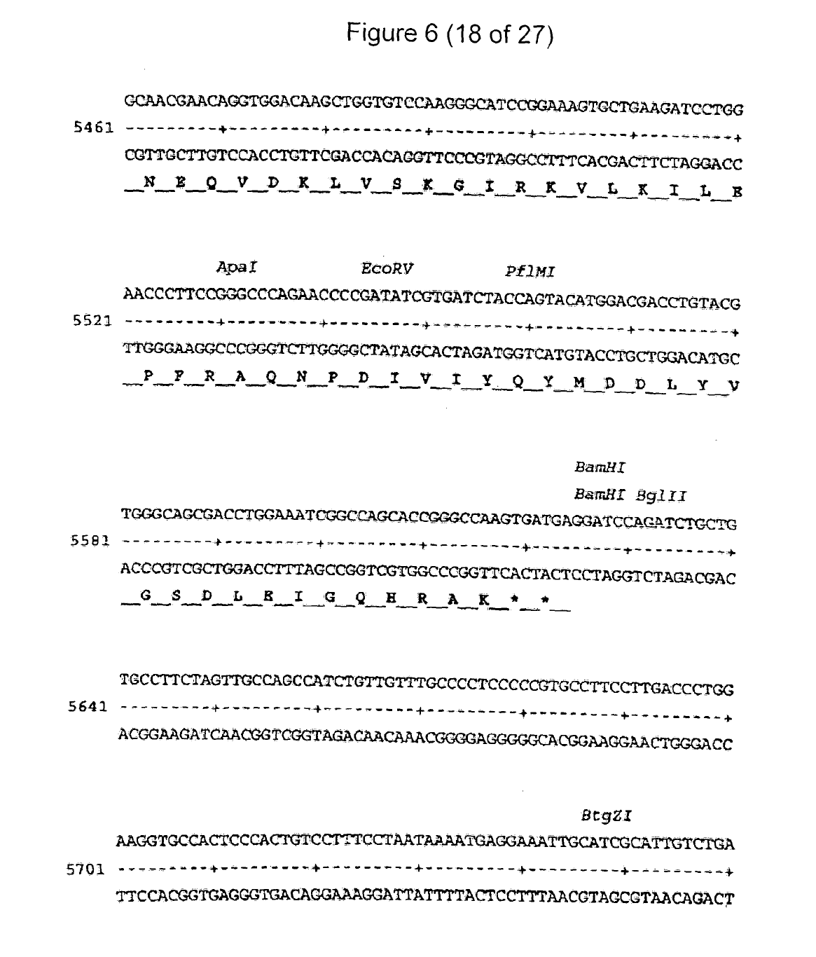

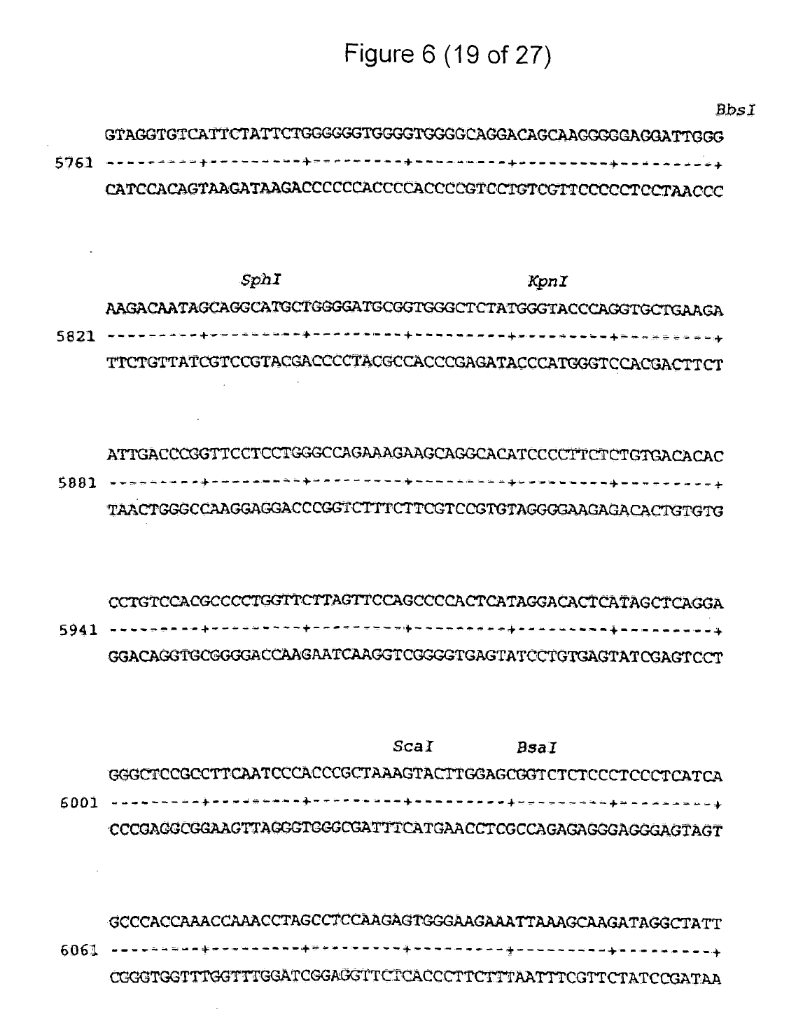

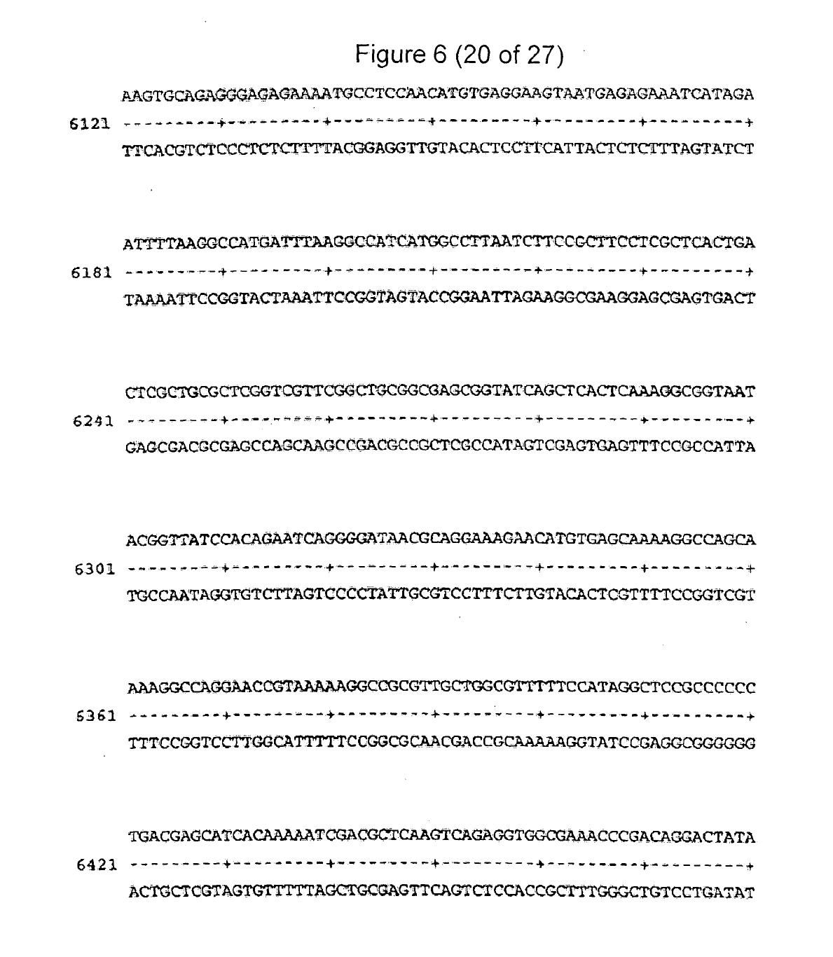

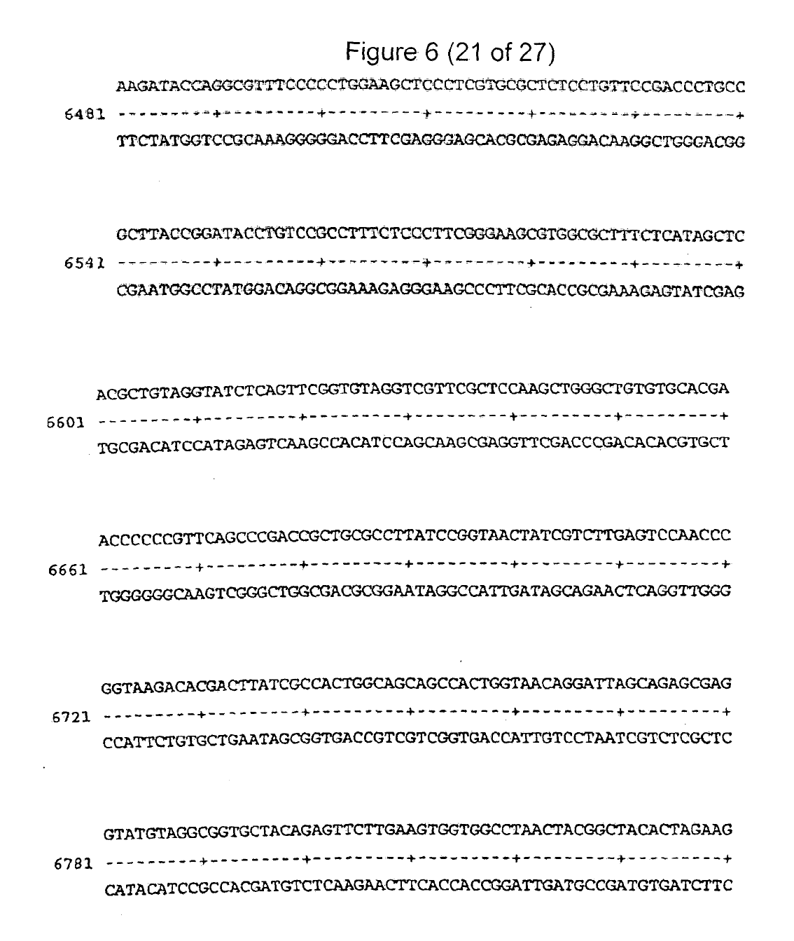

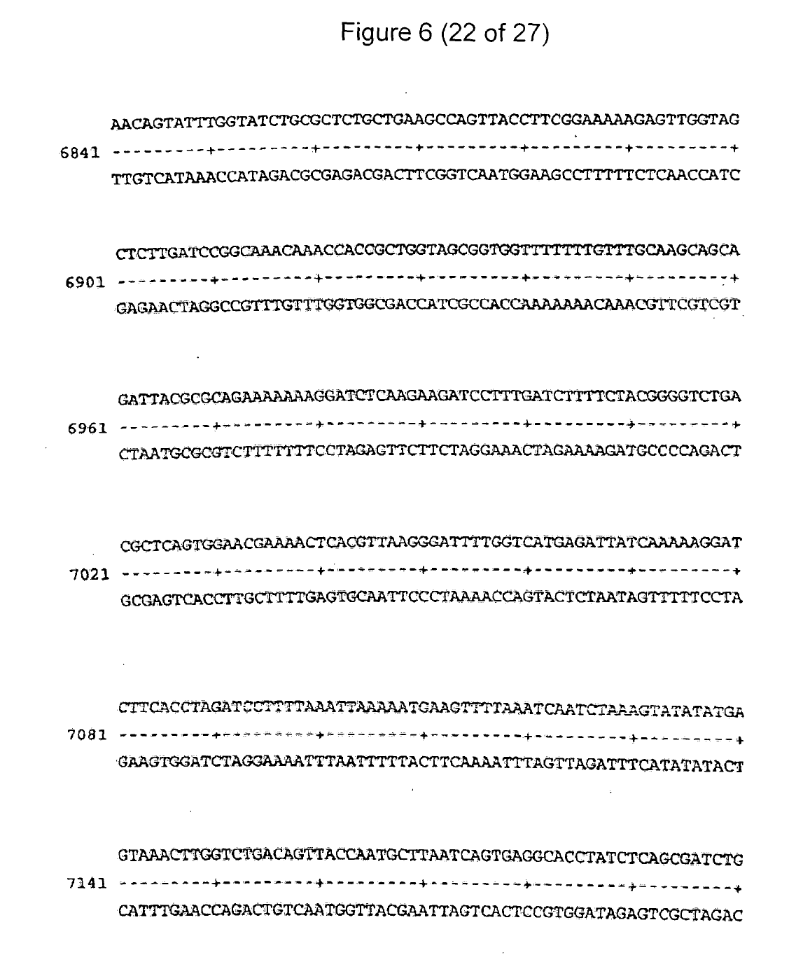

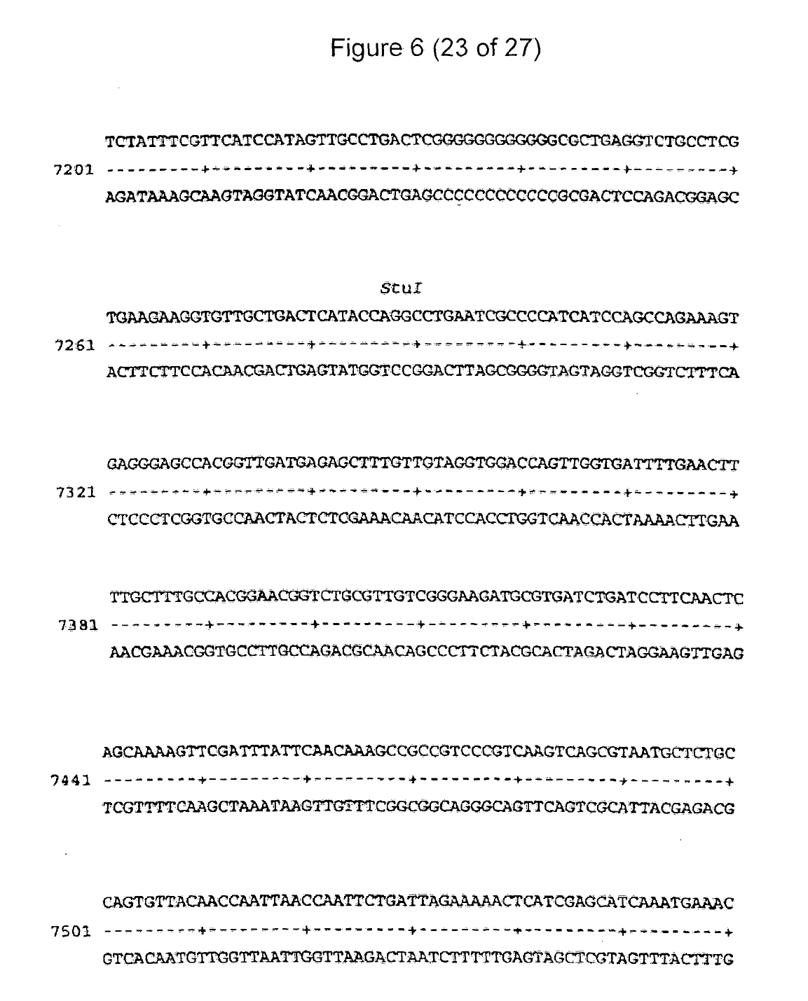

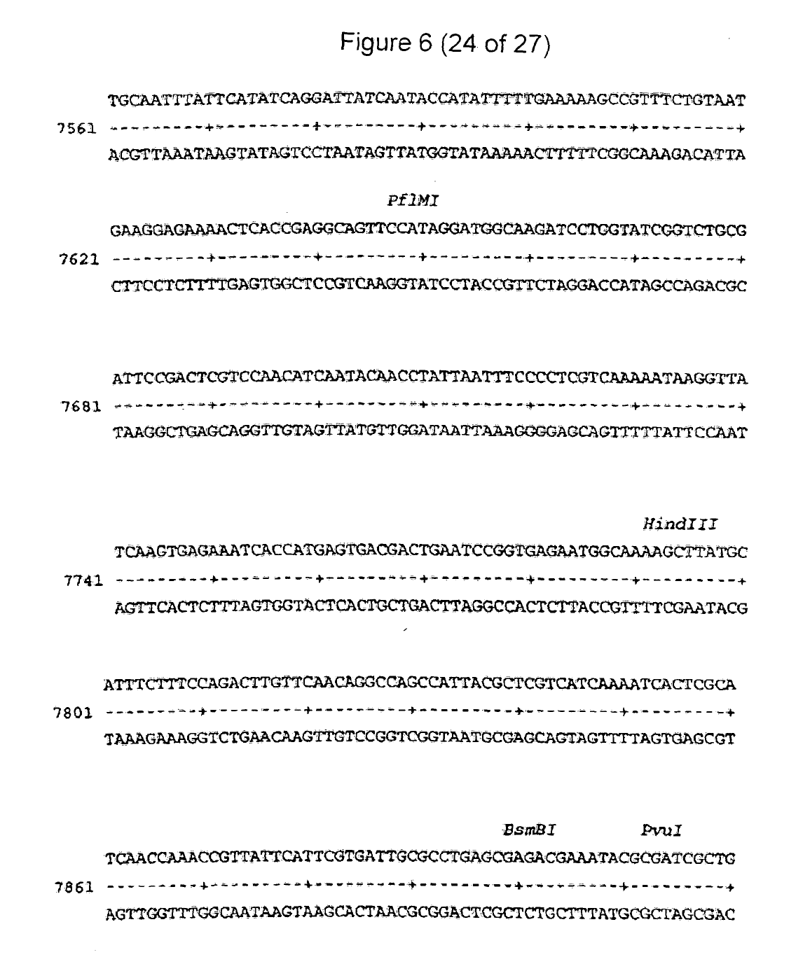

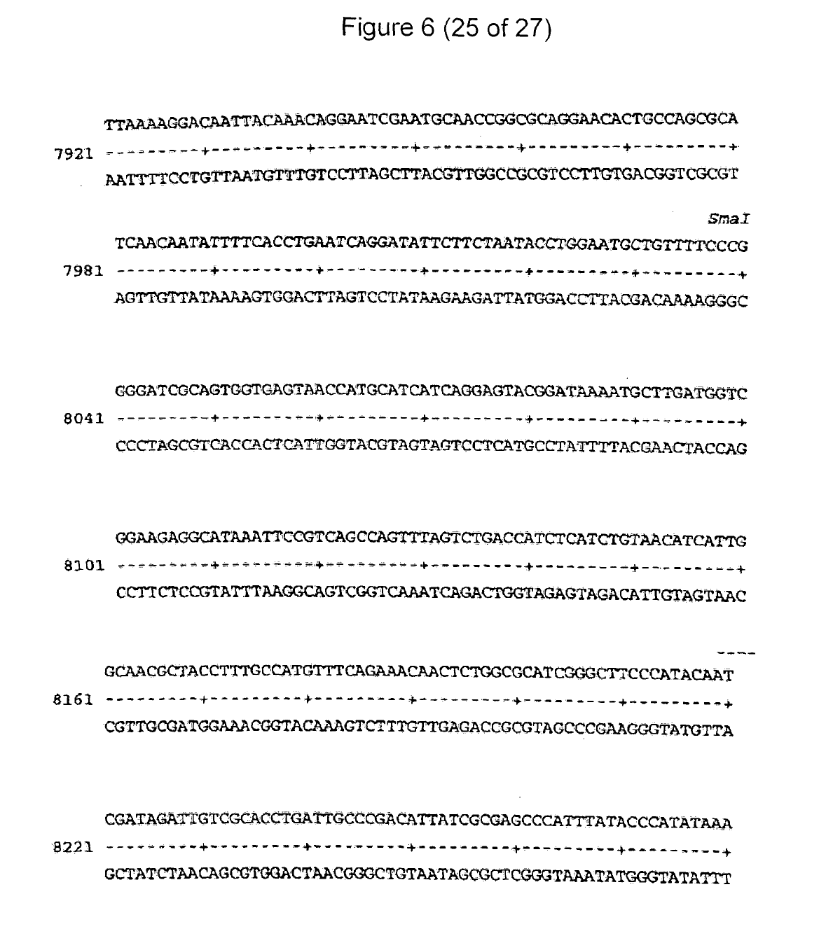

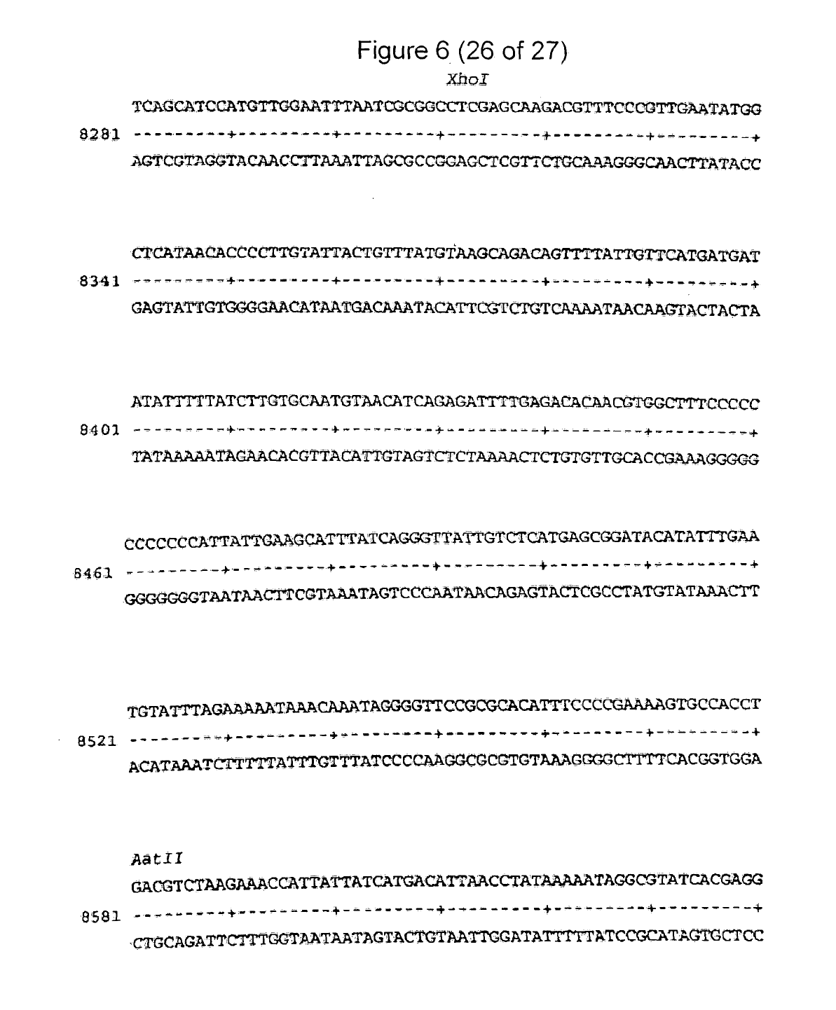

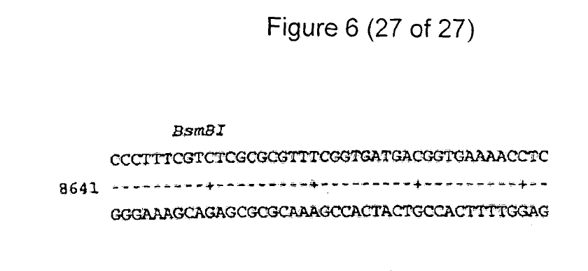

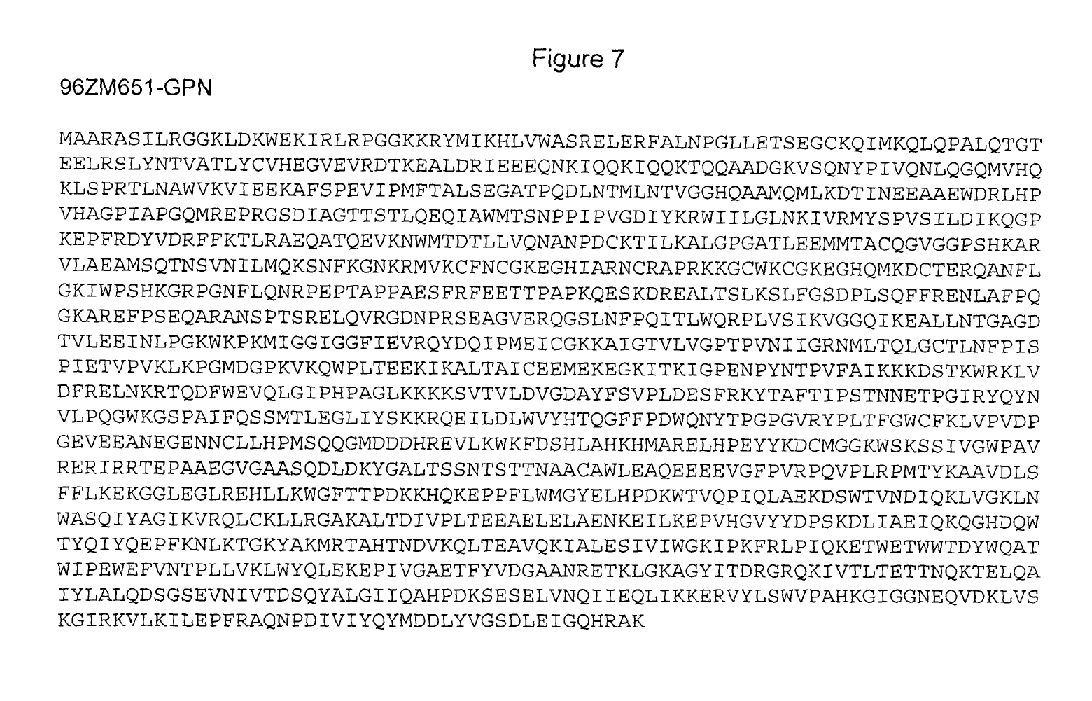

In a preferred embodiment, in step (a) naked DNA is administered two, three, four or more times to the individual with at least three weeks between each administration. Three weeks appears to be the minimum time for the naked DNA to prime an antibody response and a T cell response which is manifested after the boost with the viral vector as in step (b). The timing between administrations may be four or five or six or more weeks, and may be up to one year. It is preferred if the time between administrations is three or four weeks. It is preferred that when naked DNA is used in step (a) three separate administrations are used at least three weeks apart. A particularly preferred method of carrying out step (a) is to administer three separate doses of plasmid DNA with four weeks between the doses, wherein each dose contains a plasmid encoding the Env protein (in particular gp140) and a plasmid encoding the Gag, Pol and Nef proteins (see FIG. 1). Particularly preferred naked DNA which encodes HIV Env, and HIV Gag, Pol and Nef proteins is described in Example 1 and FIGS. 6 and 7.

When the DNA used in step (a) is naked DNA it is preferred that each individual administration event contains up to about 20 mg, and typically each dose contains >1 mg of naked DNA, typically between 1 and 5 mg. Conveniently 1 or 2 or 3 or 4 or 5 mg naked DNA may be administered in each administration event. Typically, for each administration event (ie when the individual presents on a day for administration) 2×4 mg of naked DNA is used, typically administered in different sites, such as each arm. The amount of DNA (naked DNA or DNA comprised in a viral vector) used may be selected to have a priming effect on the immune system, and the amount may vary depending on the route of administration. For example, if electroporation is used (see below) a lower dose may be required than if it is injected intra-muscularly. The concentration of naked DNA that is administered is such that the solution is not too viscous for administration, and typically is less that 4 mg DNA/ml.

For the avoidance of doubt, when we refer to dose we mean the amount (of DNA, viral vector or oligomeric gp140 protein, as the case may be) administered to the individual at substantially the same time. For example, the individual may be administered separately (eg in separate areas of the body such as in each arm) 2×4 mg within a short period of time of 15 minutes. In these circumstances the dose is deemed to be 8 mg.

When the DNA is comprised in an adenovirus vector, it is convenient that between 10⁹ and 10¹¹ viral particles (for example plaque forming units; PFU) are administered in a dose, preferably in a dose of around 10¹⁰ viral particles. As for the administration of naked DNA it may be convenient to administer the dose at different sites.

The use of DNA, particularly naked DNA, such as plasmid DNA is preferred in step (a).

Although the DNA in step (a) may be administered to the individual by any convenient route such as intra-muscularly, intra-dermally, trans-cutaneously or mucosally, or by electroporation, it is preferred if the DNA is administered intra-muscularly on each occasion.

Typically the time between the last administration to the individual of DNA or SFV encoding an HIV Env protein and optionally nucleic acid encoding one, any two or all three of the HIV Gag, Pol and Nef proteins (step (a)), and the first administration to the individual of viral vector encoding an HIV Env protein and optionally viral vector encoding one, any two or all three of the HIV Gag, Pol and Nef proteins (step (b)) is around one month, but may be longer.

In step (b) it is preferred if the viral vector is a pox virus vector, such as a vaccinia vector or fowlpox viral vector. Preferably, the viral vector is a non-replicating viral vector. Suitable viral vectors include the attenuated vaccinia virus strains MVA and NYVAC as described in Gómez et al (2007) Vaccine 25, 1969-1972, incorporated herein by reference.

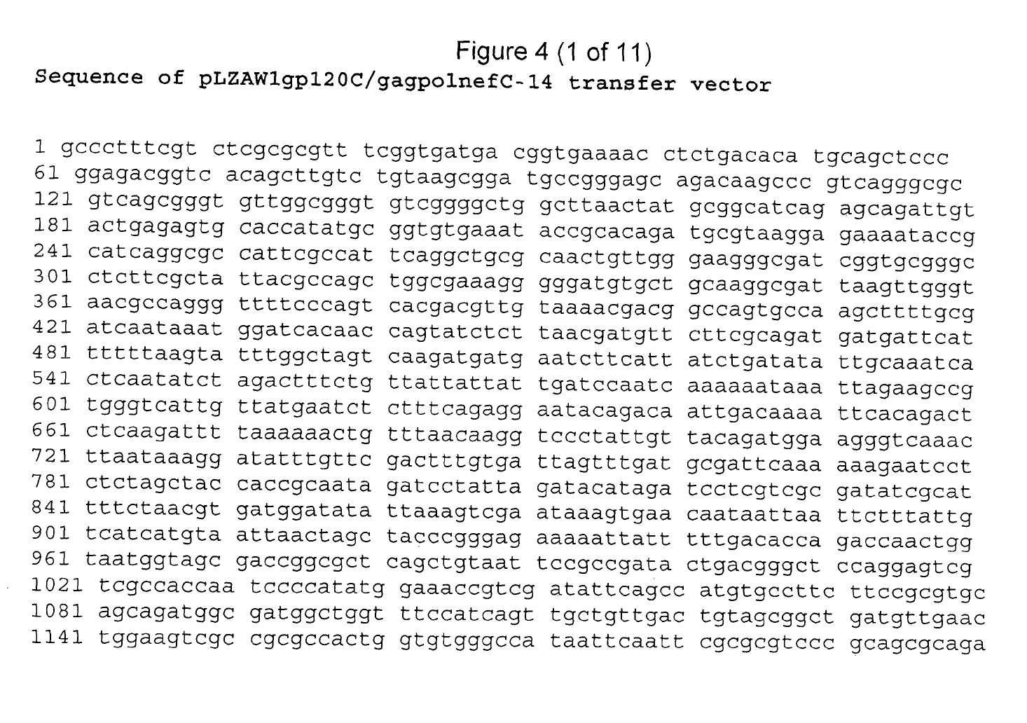

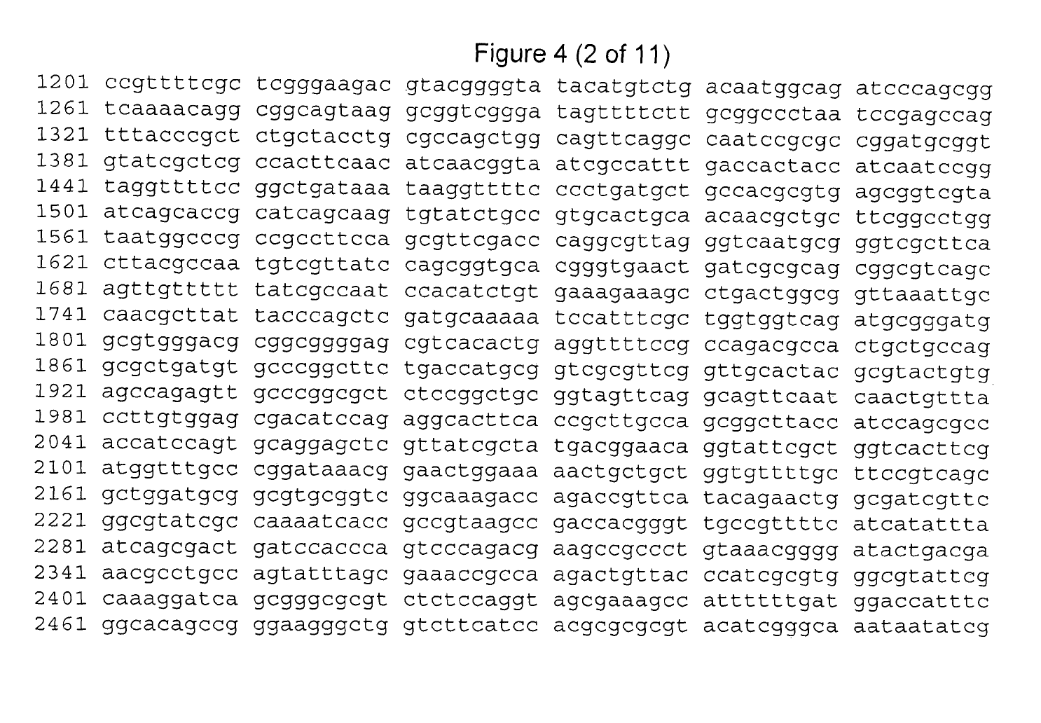

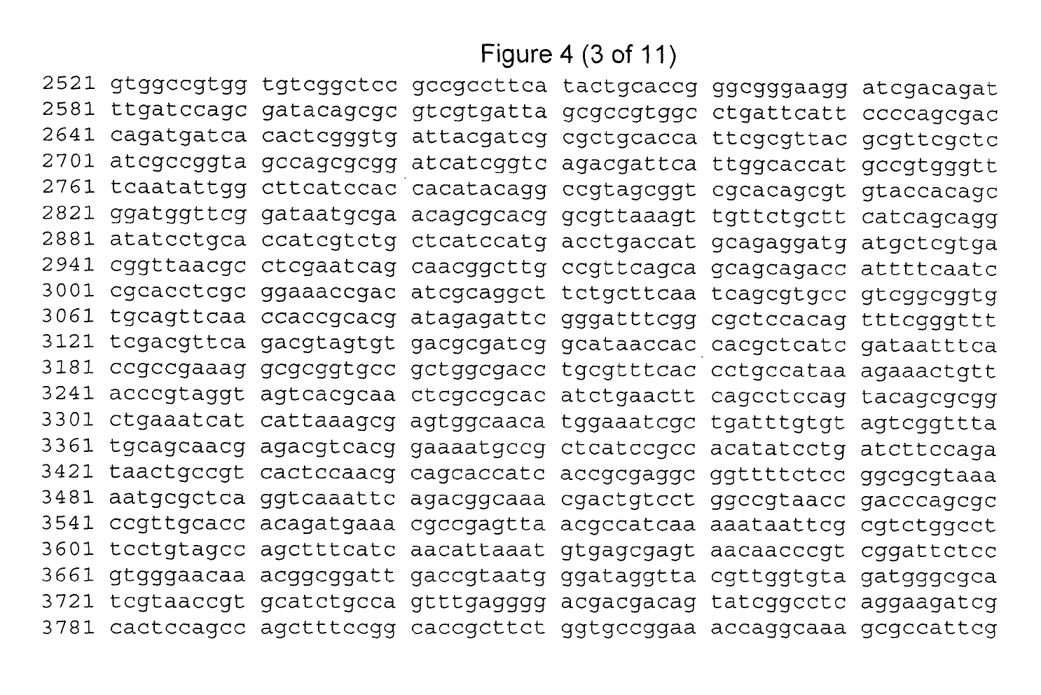

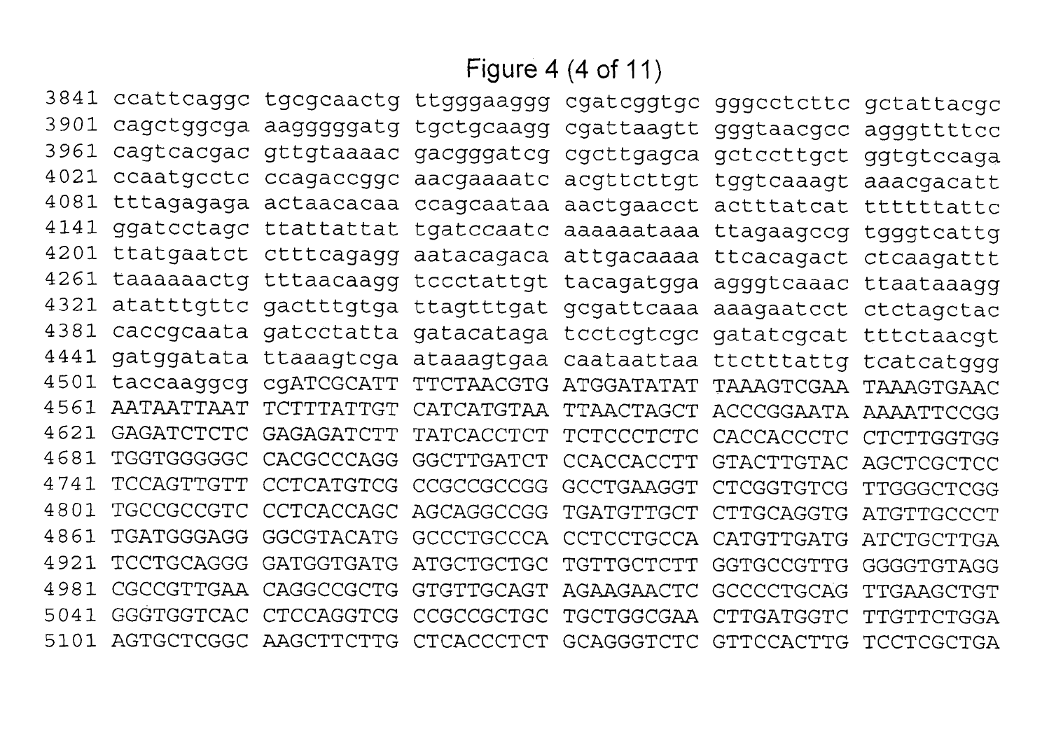

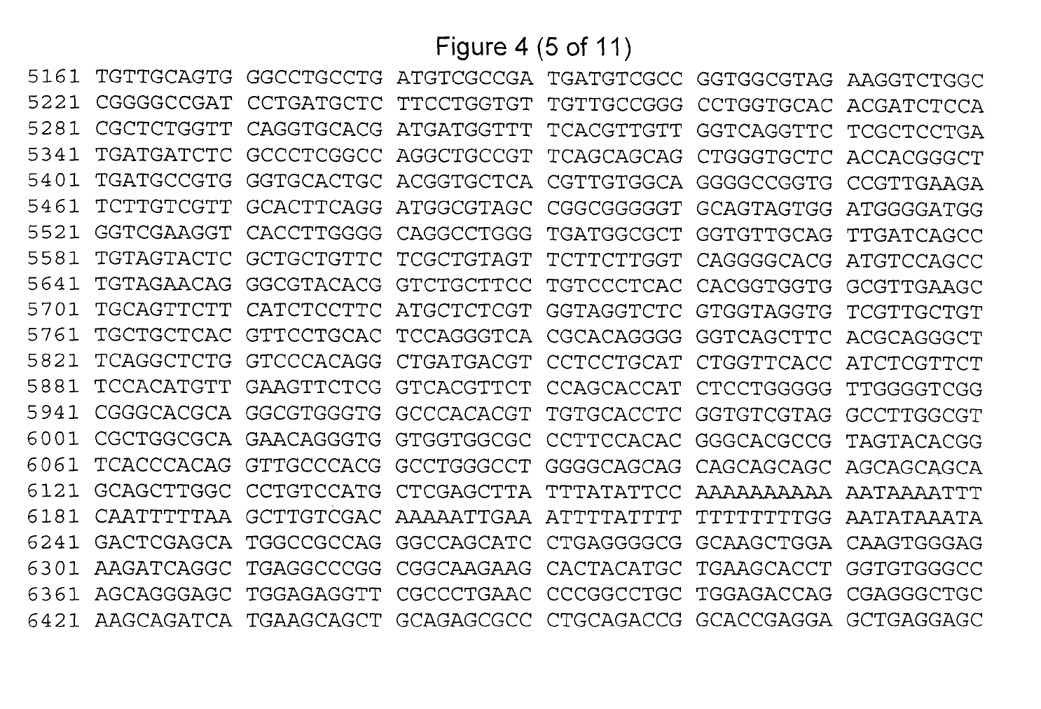

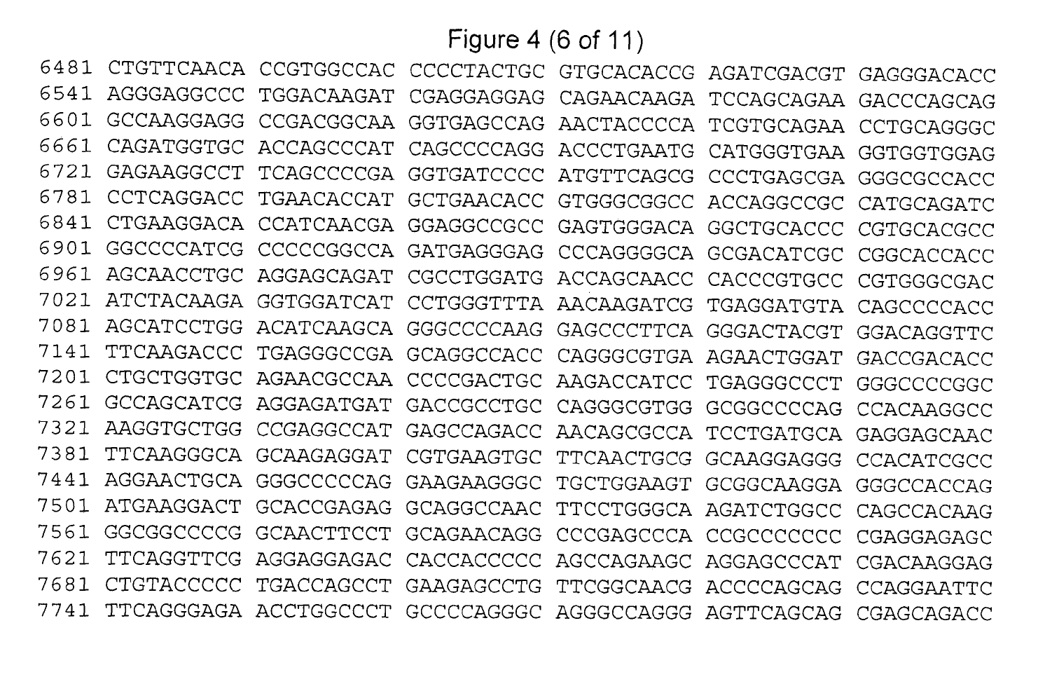

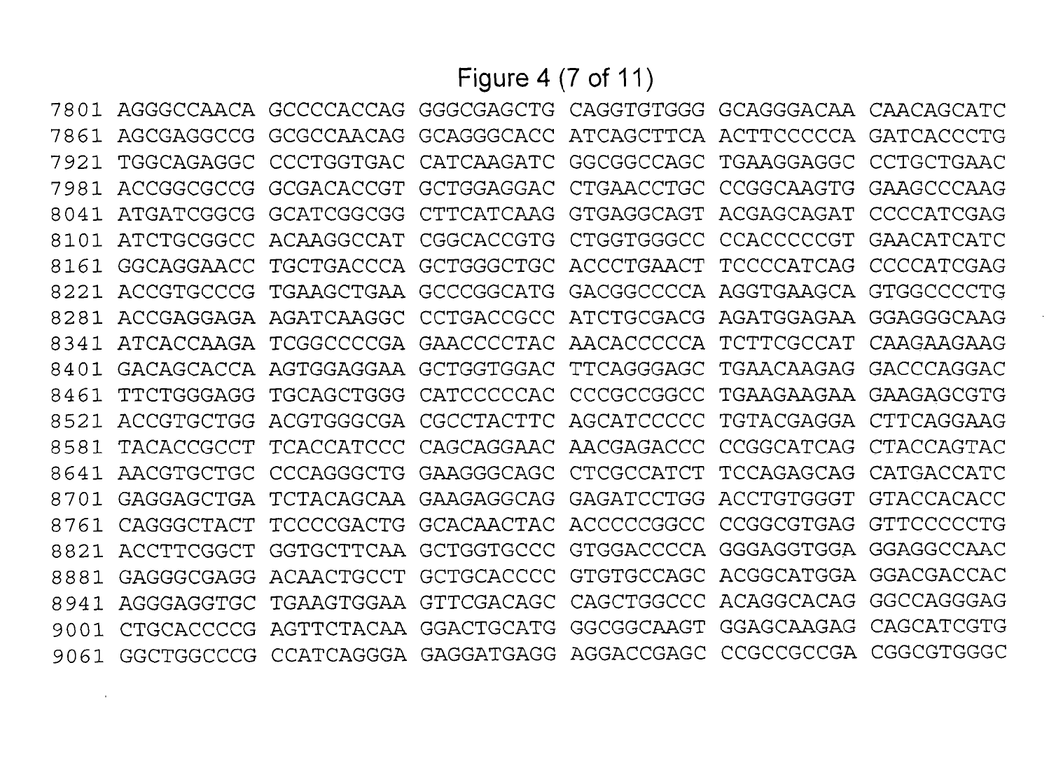

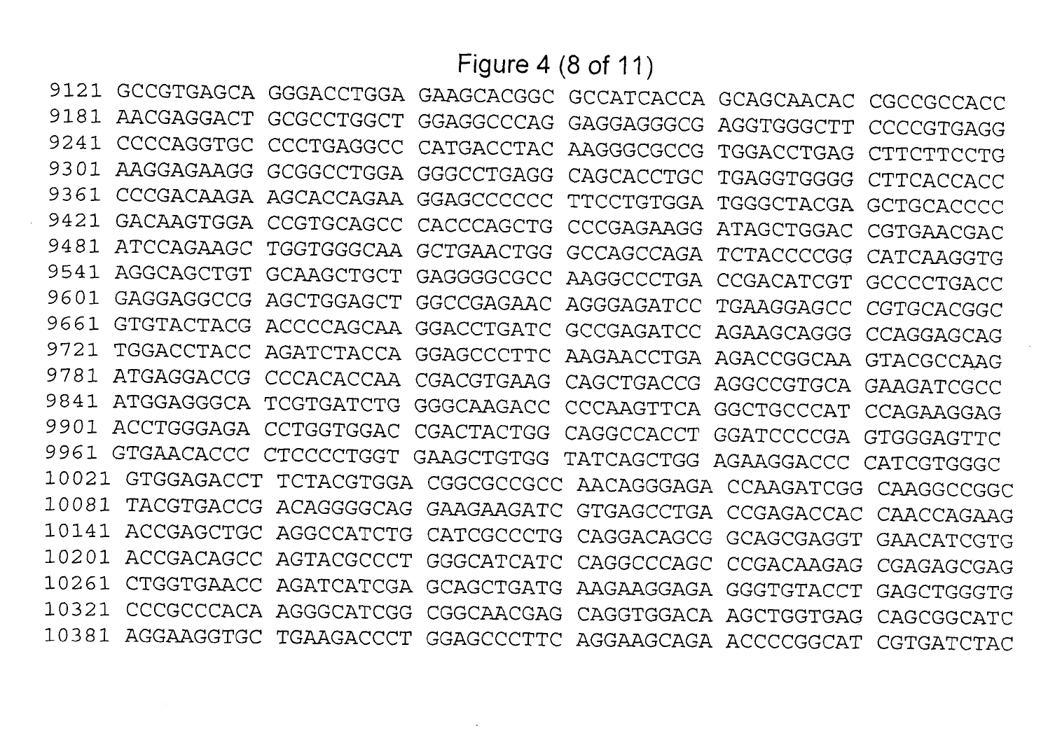

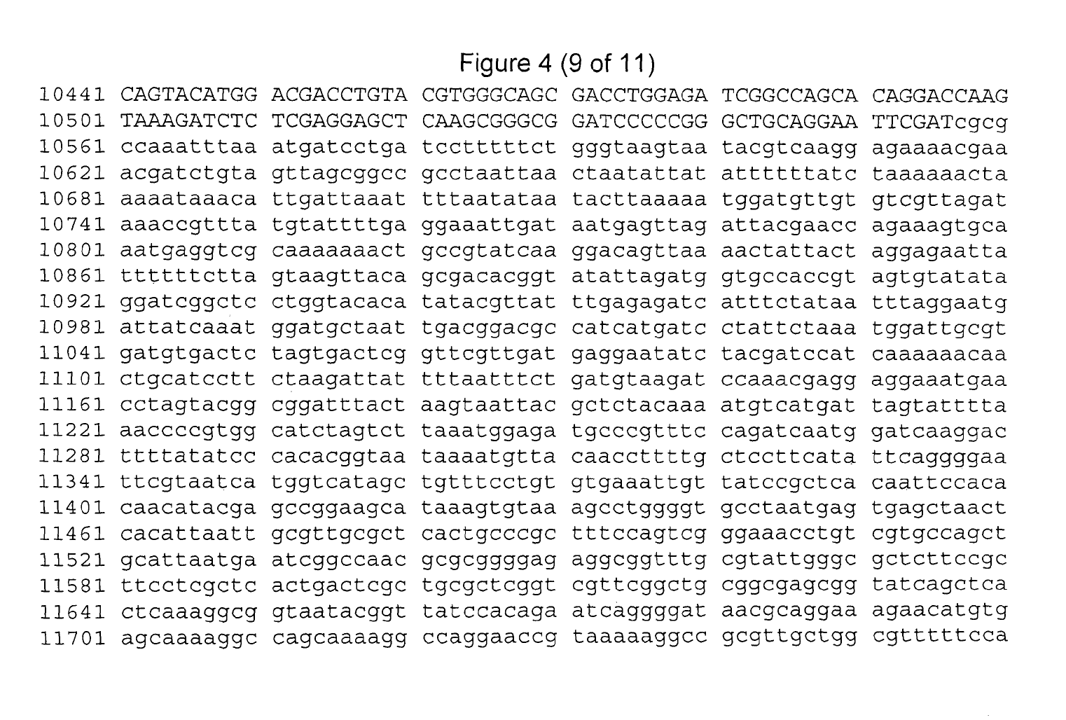

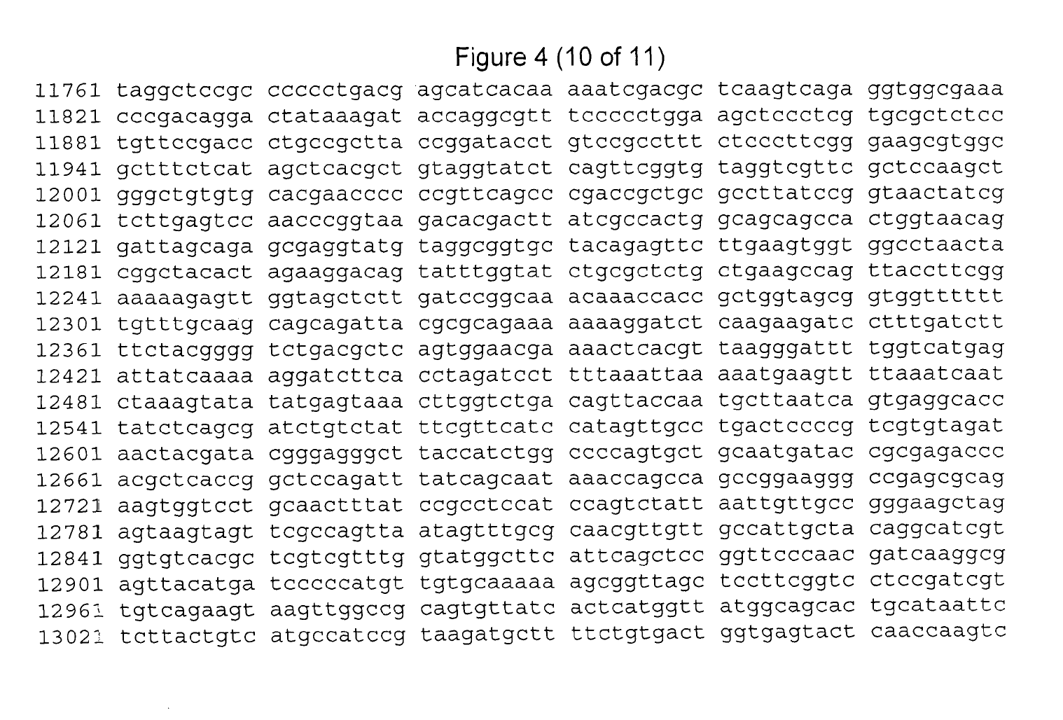

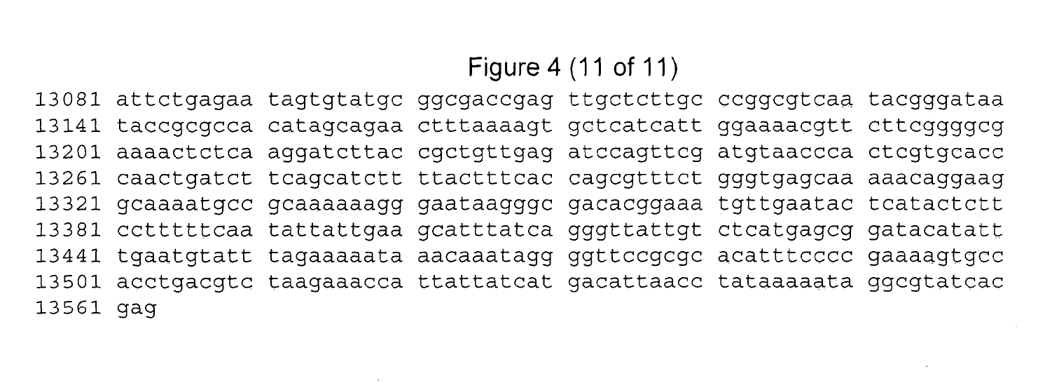

In step (b) viral vector encoding one, any two or all three of the HIV Gag, Pol and Nef proteins, as well as the HIV Env protein, may usefully be present and may be on the same or different viral vectors. Thus, in step (b) viral vector encoding Env alone or Env+Gag or Env+Pol or Env+Nef or Env+Gag+Pol or Env+Gag+Nef and so one may be used. In step (b) one, two, three or four separate viral vectors may be used, typically simultaneously. It is preferred that a single viral vector encodes all of the HIV proteins whose coding region is to be introduced into the individual in step (b). It is particularly preferred that a single viral vector that encodes each of the Env, Gag, Pol and Nef proteins is administered to the individual. It is preferred if this vector is a pox virus vector, particularly a vaccinia vector. A particular preferred vector that encodes each of Env, Gag, Poi and Nef is described in Example 1 and FIGS. 4, 5 and 16. The vector is one that is suitable for human use, and can act as a delivery system for genes expressing the polypeptides.

In a further embodiment, in step (b) the viral vector is an adenovirus or adeno-associated virus vector. The preferences for the adenovirus or adeno-associated virus vector in step (b) are the same as in step (a).

In a preferred embodiment, in step (b) viral DNA is administered one, two, or three or more times to the individual with at least three weeks, and preferably at least four weeks between each administration (if administered more than once), for example up to 12 weeks. Preferable the time between each administration is 3 or 4 weeks. It is preferred that in step (b) two separate administrations are used with at least three weeks apart. A particularly preferred method of carrying out step (b) is to administer two separate doses of pox virus vector with four weeks between the doses, wherein each dose contains a pox virus vector encoding the Env, Gag, Pol and Nef proteins (see FIG. 1). In a preferred embodiment, this follows a step (a) in which three doses of naked DNA have been administered, preferably with at least three weeks between the doses (see FIG. 1).

When the viral vector in step (b) is a pox virus vector (such as a vaccinia vector, including MVA), it is convenient that between 10⁷ and 10⁹ TCIDs (tissue culture infection doses) are administered in a dose. A preferred dose is around 10⁸ TCIDs. When the viral vector in step (b) is an adenoviral vector, it is convenient that the dose is between 10⁹ and 10¹¹, preferably around 10¹⁰ viral particles (for example PFUs). As for the administration of DNA in step (a) it may be convenient to administer the dose of viral vector instep (b) at different sites.

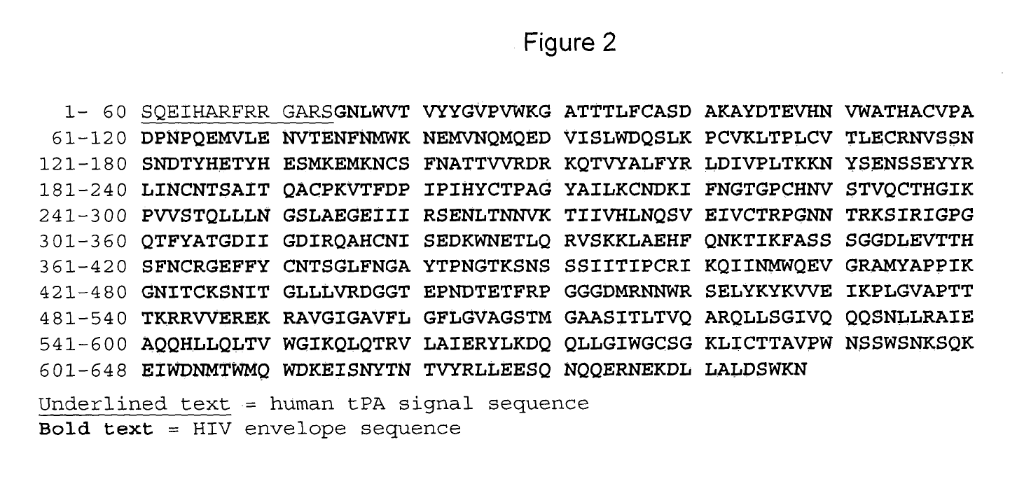

In step (c) the HIV Env protein is oligomeric, and typically, the Env protein has a trimeric structure. An oligomeric structure of Env protein may be obtained by retention of the Env oligomerisation domain. Advantageously, the oligomeric HIV Env protein retains the same configuration as, or a configuration that is immunologically similar to, the Env protein in native HIV. The oligomeric (eg trimeric) form of the HIV Env protein can be assessed by polyacrylamide gel electrophoresis. Conveniently, the oligomeric HIV Env protein is post-translationally modified, such as glycosylated, for example by recombinant expression in CHO cells. Typically, the glycosylation of the oligomeric Env protein is similar to that of native HIV. Particularly preferred HIV Env protein is described in Example 1 and in FIG. 2.

It is preferred that in each of steps (a), (b) and (c) the HIV Env protein, and when it is present, the HIV Gag, Pol and Nef proteins have substantially the same amino acid sequence. For example, there may be complete identity or 1, 2, 3, 4, 5 or 6 amino acid differences. For example, up to 5 or 10% of the amino acids may be different. In other words, it is preferred that the Env protein is homologous in each of steps (a), (b) and (c), and it is also preferred if the Gag, Pol and Nef proteins (if present) are homologous in steps (a) and (b). It is not necessary for the HIV proteins to be identical to each other; for example, in steps (a) and (b) gp120, which cannot trimerise because it lacks the relevant protein domain, may be used and gp140 used in step (c), but the overlapping sequence is preferably identical. However, it is preferred that at least the Gag, Pol and Nef proteins encoded by the DNA in step (a) and the viral vector in step (b) are identical.

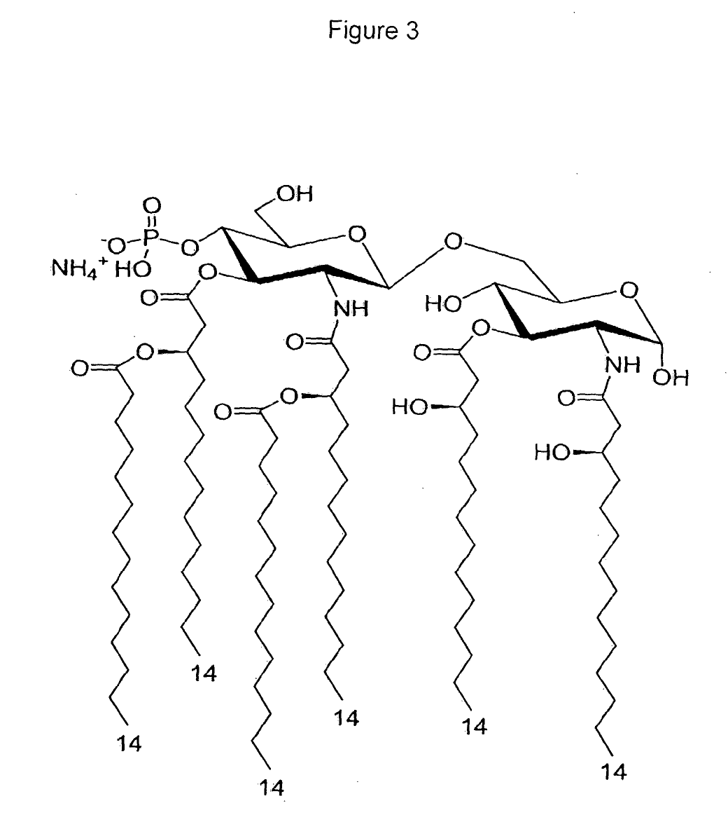

Alum alone is not a preferred adjuvant for use in step (c) because it may not be sufficiently potent in stimulating an immune response. Preferably, the adjuvant is one which is more potent than alum. Typically, it has a potency of at least 1.5 or 2 or 3 or 4 or more times that of alum on a weight basis. Suitable adjuvants may be selected from the group consisting of glucopyranosyl lipid A (such as GLA), AS02 and AS04. AS02 and AS04 are proprietary adjuvants of GlaxoSmithKline (GSK). AS02 contains MPL™ and QS-21 in an oil-in-water emulsion and is described in EP 0 671 948, EP 0 761 231 and U.S. Pat. No. 5,750,110, incorporated herein by reference. AS04 also is composed of MPL, but in combination with alum. It is described in EP 1 126 876 and U.S. Pat. No. 7,357,936, incorporated herein by reference. MPL is composed of a series of 4′-monophosphoryl lipid A species that vary in the extent and position of fatty acid substitution. It is prepared from lipopolysaccharide (LPS) of Salmonella minnesota R595 by treating LPS with mild acid and base hydrolysis followed by purification of the modified LPS. MPL is described in EP 0 971 739, EP 1 194 166 and U.S. Pat. No. 6,491,919, incorporated herein by reference. QS-21 is a natural product of the bark of the Quillaja saponaria tree species, and is described in EP 0 606 317 and U.S. Pat. No. 5,583,112, incorporated herein by reference. GLA, and in particular the aqueous formulation GLA-AF, is a preferred adjuvant and is described in Example 1 and FIG. 3. In one embodiment the adjuvant contains a component that binds to the Toll-like receptor 4 (TLR4).

Preferably, between 10 μg and 1 mg of the Env protein is used, for example between 50 μg and 500 μg such as 100 μg. Preferably between 1 μg and 50 μg of GLA-AF adjuvant is used, for example between 3 μg and 10 μg such as 5 μg. In a preferred embodiment 100 μg of gp140 and 5 μg of GLA-AF are used. The amounts of Env protein and adjuvant used are suitable for human administration.

Steps (b) and (c) may be carried out simultaneously. This is particularly preferred. For the avoidance of doubt, if steps (b) and (c) are carried out simultaneously (typically by administering the viral vector and Env/adjuvant components essentially at the same time but in two different sites in the individual, for example in each arm), the combined step may be repeated after a suitable time period. Thus, it is particularly preferred if the viral vector encoding an HIV Env protein and optionally nucleic acid encoding one, any two or all three of the HIV Gag, Pol and Nef proteins, is administered at one site in the individual, and that the oligomeric Env protein and the adjuvant are combined and administered at another site in the individual, typically with around 15 minutes of each other, and that this regime of administration may be repeated more than once after step (a). However, it is preferred if the individual is administered only one dose of viral vector and Env/adjuvant in steps (b) and (c). Typically, the adjuvanted Env protein is stored before use at a temperature of no more than 4° C. For example, the GP140-GLA mix can be stored at room temperature for up to 8 hours, though we recommend no more than 2 hours. GLA and gp140 can typically be stored unmixed between 2-8° C. This is necessary for GLA, though gp140 can be stored at −20° C. or <=−55° C. Typically the viral vector is stored before use as a temperature of no more than −20° C. Clearly, if frozen, the components are thawed before use.

We have found that when combining Env protein with adjuvant (eg gp140+GLA-AF) with immunisation using the viral vector (eg MVA-C) the adjuvant not only provides an adjuvant effect for the Env protein (eg gp140) but also an adjuvant effect for the viral vector (eg MVA-C) when administered at a different site in the individual. The invention therefore includes the simultaneous use of Env protein plus adjuvant (eg gp140 plus GLA-AF) and viral vector (eg MVA-C) administered at separate sites that essentially at the same time, as well as the simultaneous use of Env protein plus adjuvant (eg gp140 plus GLA-AF) and viral vector plus adjuvant (eg MVA-C plus GLA-AF) administered at separate sites but essentially at the same time. This takes place after step (a).

A particularly preferred method of the invention is a method of raising an immune response in an individual against HIV the method comprising:
(1) administering to the individual a naked DNA molecule (such as a plasmid) encoding an HIV Env protein, and a naked DNA molecule (such as a plasmid) encoding all three of the HIV Gag, Pol and Nef proteins; and optionally repeating the administration up to two more times with three to four weeks between each administrations;
(2) after a period of at least four weeks (for example up to 12 weeks, suitably eight weeks) administering to the individual vaccinia vector encoding a HIV Env Gag, Pot and Nef proteins, and oligomeric HIV Env protein adjuvanted with an adjuvant (preferably other than alum, such as GLA-AF); and optionally repeating the administration once more with around three to four weeks between the administrations. In this way, a significant reduction in the overall length of the treatment regime may be achieved.

A further particularly preferred method of the invention is a method of raising an immune response in an individual against HIV the method comprising:
(1) administering to the individual an adenovirus or adeno-associated virus vector encoding an HIV Env protein, and optionally (and preferably) also encoding all three of the HIV Gag, Pol and Nef proteins; and optionally repeating the administration up to one more time with three to four weeks between each administrations;
(2) after a period of at least four weeks (for example up to 12 weeks, suitably eight weeks) administering to the individual an adenovirus or adeno-associated virus vector encoding an HIV Env protein, and optionally (and preferably) also encoding all three of the HIV Gag, Pol and Nef proteins, and oligomeric HIV Env protein adjuvanted with an adjuvant (preferably other than alum, such as GLA-AF); and optionally repeating the administration once more with around three to four weeks between the administrations. Preferably, in step (1) the administration is not repeated. Preferably, in step (2) the administration is not repeated. In this way, a significant reduction in the overall length of the treatment regime may be achieved.

A further particularly preferred method of the invention is a method of raising an immune response in an individual against HIV the method comprising:
(1) administering to the individual a SFV encoding an HIV Env protein, and optionally (and preferably) also encoding all three of the HIV Gag, Pol and Nef proteins; and optionally repeating the administration up to one more time with three to four weeks between each administrations;
(2) after a period of at least four weeks (for example up to 12 weeks, suitably eight weeks) administering to the individual vaccinia vector encoding a HIV Env Gag, Pol and Nef proteins, and oligomeric HIV Env protein adjuvanted with an adjuvant (preferably other than alum, such as GLA-AF); and optionally repeating the administration once more with around three to four weeks between the administrations. Preferably, in step (1) the administration is not repeated. Preferably, in step (2) the administration is not repeated. In this way, a significant reduction in the overall length of the treatment regime may be achieved.

It will be appreciated that in the above three preferred methods, step (1) is a step (a), and step (2) is a combination of steps (b) and (c).

Preferred treatment protocols are shown in the following table 1, with reference to the steps of the method set forth in the first aspect of the invention:

TABLE 1

| Step (a) | Step (b) | Step (c) |
|---|---|---|
| DNA | MVA | gp140/adjuvant |
| DNA | MVA | gp140/adjuvant |
| DNA | | |
| DNA | MVA-gp140/adjuvant | |
| DNA | MVA-gp140/adjuvant | |
| DNA | (ie steps (b) and (c) combined | |
| DNA | SAd | gp140/adjuvant |
| DNA | SAd | gp140/adjuvant |
| DNA | | |
| DNA | SAd-gp140/adjuvant | |
| DNA | SAd-gp140/adjuvant | |
| DNA | (ie steps (b) and (c) combined) | |
| SAd | SAd | gp140/adjuvant |
| | | gp140/adjuvant |

| | |
|---|---|
| SAd | SAd-gp140/adjuvant |
| | (ie steps (b) and (c) combined) |
| SAd | MVA-gp140/adjuvant |
| | (ie steps (b) and (c) combined) |
| SFV | MVA-gp140/adjuvant |
| | (ie steps (b) and (c) combined) |

In the above table, “DNA” means naked DNA; “SAd” means DNA comprised in an adenovirus or adeno-associated virus vector, especially of simian origin; SFV, means Semliki Forest virus vector; MVA means a pox viral vector especially a vaccinia vector such as the MVA vector described in the examples; and gp140 means an oligomeric Env protein, especially gp140 such as described in the examples. As can be seen SAd may be used in step (a) or step (b) or both steps. In step (b) the SAd may be combined with the gp140/adjuvant.

The following regimes are particularly preferred: DNA×3, MVA×2, gp140×2 sequentially; DNA×3, MVA/gp140×2 sequentially; SAd×1 (or ×2), MVA×1 (or ×2), gp140×2; SAd×1, MVA/gp140×1 (or ×2); and SFV×1, MVA/gp140×1 (or ×2).

Of course, other options are possible, for example SFV-Ad-gp140; DNA-SFV-gp140.

It is preferred that in all embodiments, unless expressly stated, that only one or two different DNAs (step (a)) or viral vectors (step (b)) are used, and it is preferred that they encode HIV proteins from the same clade, and preferably from the same variant, as discussed above. In other words, the preferred methods do not require the administration of multiple different DNA or viral vectors, and the preferred methods do not require the administration of DNA or viral vector or proteins relating to multiple clades.

It is preferred that no other HIV proteins, or DNA or viral vector with coding regions other than Env and optionally one, any two or all three of the HIV Gag, Pol and Nef proteins, are administered to the patient. It is preferred if reverse transcriptase (RT) protein or DNA or viral vector with a coding region for RT are not administered. The presence of Nef protein or coding regions is less preferred.

A second aspect of the invention provides DNA or SFV encoding an HIV Env protein and optionally nucleic acid encoding one, any two or all three of the HIV Gag, Pol and Nef proteins for use in raising an immune response in an individual against human immunodeficiency virus (HIV) wherein the individual is also administered viral vector encoding an HIV Env protein and optionally viral vector encoding one, any two or all three of the HIV Gag, Pol and Nef proteins; and is also administered oligomeric HIV Env protein combined with an adjuvant.

A third aspect of the invention provides viral vector encoding an HIV Env protein and optionally viral vector encoding one, any two or all three of the HIV Gag, Pol and Nef proteins for use in raising an immune response in an individual against human immunodeficiency virus (HIV) wherein the individual is also administered DNA or SFV encoding an HIV Env protein and optionally nucleic acid encoding one, any two or all three of the HIV Gag, Pol and Nef proteins; and is also administered oligomeric HIV Env protein combined with an adjuvant. The treatment regime is in accordance with the method of the first aspect of the invention.

A fourth aspect of the invention provides oligomeric HIV Env protein combined with an adjuvant for use in raising an immune response in an individual against human immunodeficiency virus (HIV) wherein the individual is also administered DNA or SFV encoding an HIV Env protein and optionally nucleic acid encoding one, any two or all three of the HIV Gag, Pol and Nef proteins; and is also administered viral vector encoding an HIV Env protein and optionally viral vector encoding one, any two or all three of the HIV Gag, Pol and Nef proteins. The treatment regime is in accordance with the method of the first aspect of the invention.

A fifth aspect of the invention provides DNA or SFV encoding an HIV Env protein and optionally nucleic acid encoding one, any two or all three of the HIV Gag, Pol and Nef proteins in the manufacture of a medicament for raising an immune response in an individual against human immunodeficiency virus (HIV) wherein the individual is also administered viral vector encoding an HIV Env protein and optionally viral vector encoding one, any two or all three of the HIV Gag, Pol and Nef proteins; and is also administered oligomeric HIV Env protein combined with an adjuvant. The treatment regime is in accordance with the method of the first aspect of the invention.

A sixth aspect of the invention provides viral vector encoding an HIV Env protein and optionally viral vector encoding one, any two or all three of the HIV Gag, Pol and Nef to proteins in the manufacture of a medicament for raising an immune response in an individual against human immunodeficiency virus (HIV) wherein the individual is also administered DNA or SFV encoding an HIV Env protein and optionally nucleic acid encoding one, any two or all three of the HIV Gag, Pol and Nef proteins; and is also administered oligomeric HIV Env protein combined with an adjuvant.

A seventh aspect of the invention provides oligomeric HIV Env protein combined with adjuvant in the manufacture of a medicament for raising an immune response in an individual against human immunodeficiency virus (HIV) wherein the individual is also administered DNA or SFV encoding an HIV Env protein and optionally nucleic acid encoding one, any two or all three of the HIV Gag, Pol and Nef proteins; and is also administered viral vector encoding an HIV Env protein and optionally viral vector encoding one, any two or all three of the HIV Gag, Pol and Nef proteins.

The treatment regimes of the second to seventh aspects of the invention are in accordance with the method of the first aspect of the invention, including in particular the order and timing of the administration of the various components.

An eighth aspect of the invention provides a composition comprising an oligomeric HIV Env protein and a GLA adjuvant. The composition is useful as part of a treatment regime for raising an immune response in an individual as discussed above.

A ninth aspect of the invention provides a kit of parts comprising an oligomeric HIV Env protein; and an adjuvant. Preferably the adjuvant is GLA. Typically, the kit of parts also contains viral vector encoding an HIV Env protein and optionally viral vector encoding one, any two or all three of the HIV Gag, Pol and Nef proteins. Conveniently, the kit of parts also contains DNA or SFV encoding an HIV Env protein and optionally nucleic acid encoding one, any two or all three of the HIV Gag, Poi and Nef protein. The kits of parts are useful for the supply of some or all of the components of a treatment regime for raising an immune response in an individual to HIV as discussed above.

Kits of parts of the invention include oligomeric HIV Env protein such as gp140 adjuvanted with an adjuvant such as GLA and a vaccinia viral vector encoding Env, Gag, Pol and Nef, optionally adjuvanted with an adjuvant such as GLA. Furtther suitable kits of parts can be derived from the information in the above table, and include each of the separate components for administration in each of the protocols described.

The preferences expressed in relation to the method of raising an immune response in an individual to HIV first aspect of the invention also apply to the further aspects of the invention.

The DNA, viral vector and Env protein/adjuvant components of the invention are prepared in pharmaceutically acceptable form, and so are sterile, and are suitable for administration to a human individual. Methods of preparing DNA, virus and protein components for pharmaceutical use are known in the art.

The DNA, viral vectors and Env protein may be prepared using well known molecular biology, protein engineering and biochemical purification methods.

The invention will now be described by reference to the following figures and Examples, none of which limit the scope of the invention.

FIG. 1 shows a schematic of particular preferred embodiments of the invention. The left-hand protocol is particularly preferred as it reduces the number of administrations of the vaccine components and shortens the overall treatment regime.

FIG. 2 shows the amino acid sequence of CN54gp140.

FIG. 3 shows the structural formula of the bulk ammonium salt form of GLA.

FIG. 4 shows the nucleotide sequence of the pLZAW1gp120C/gagpolnefC-14 transfer vector.

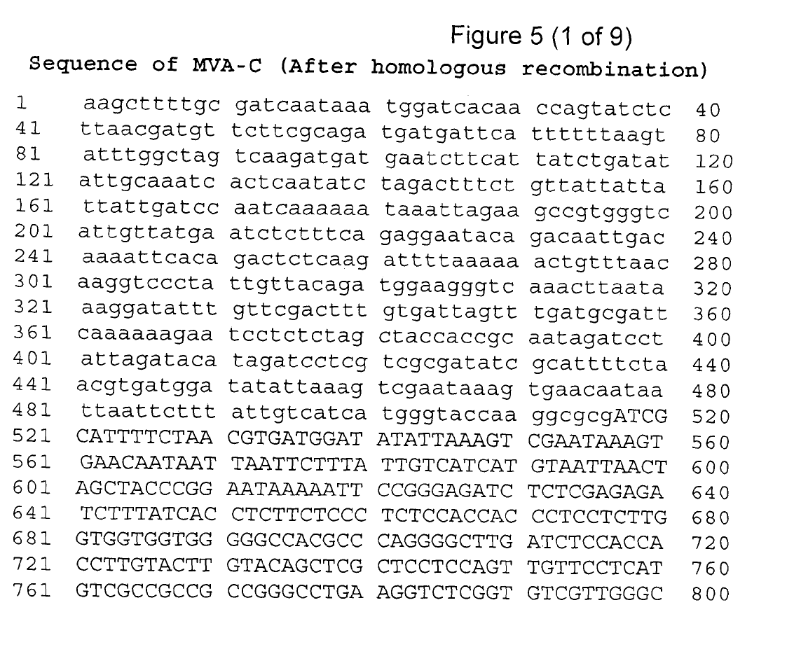

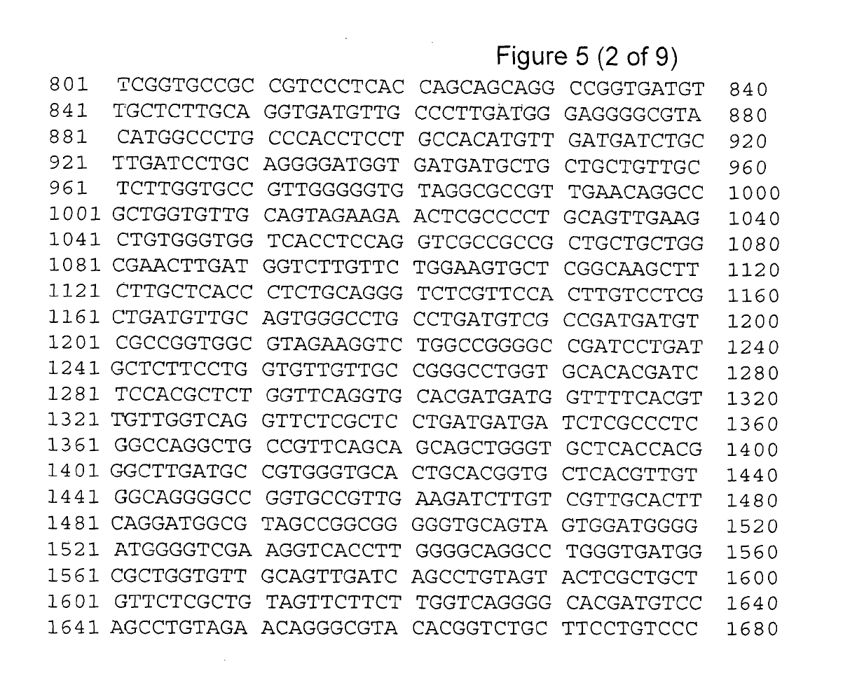

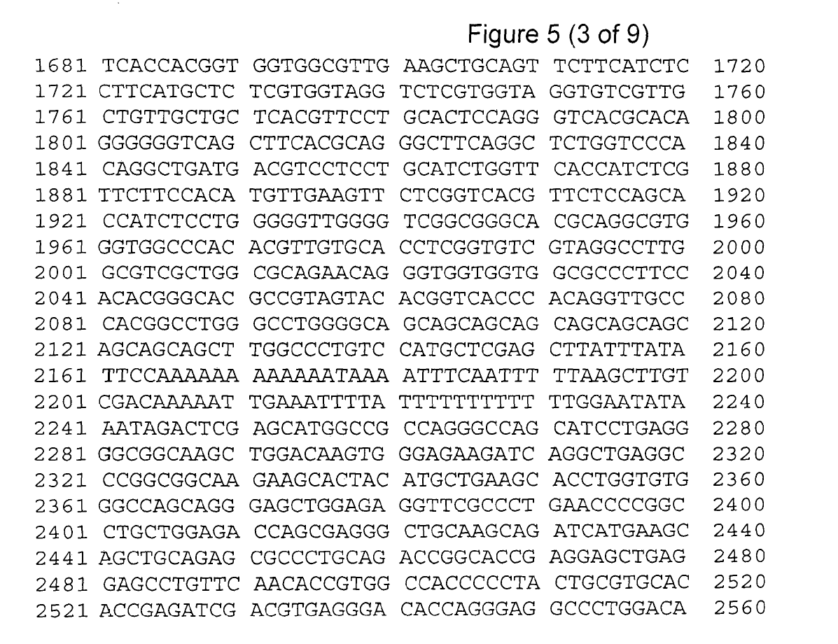

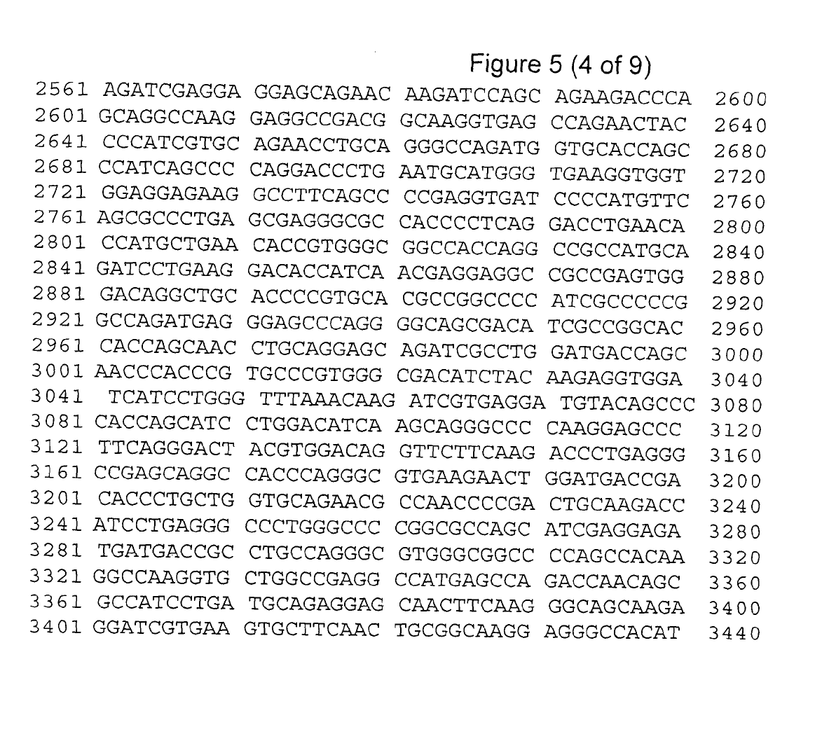

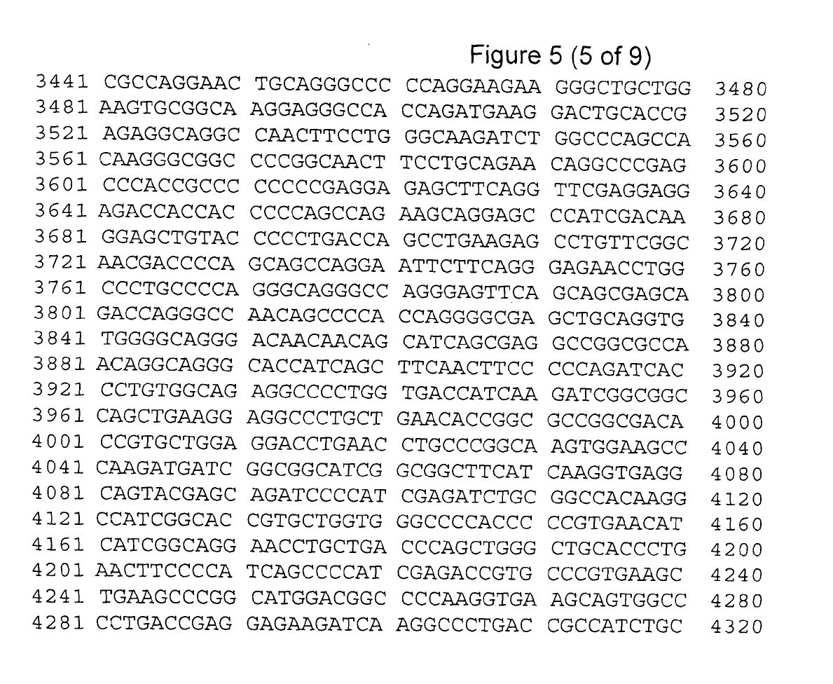

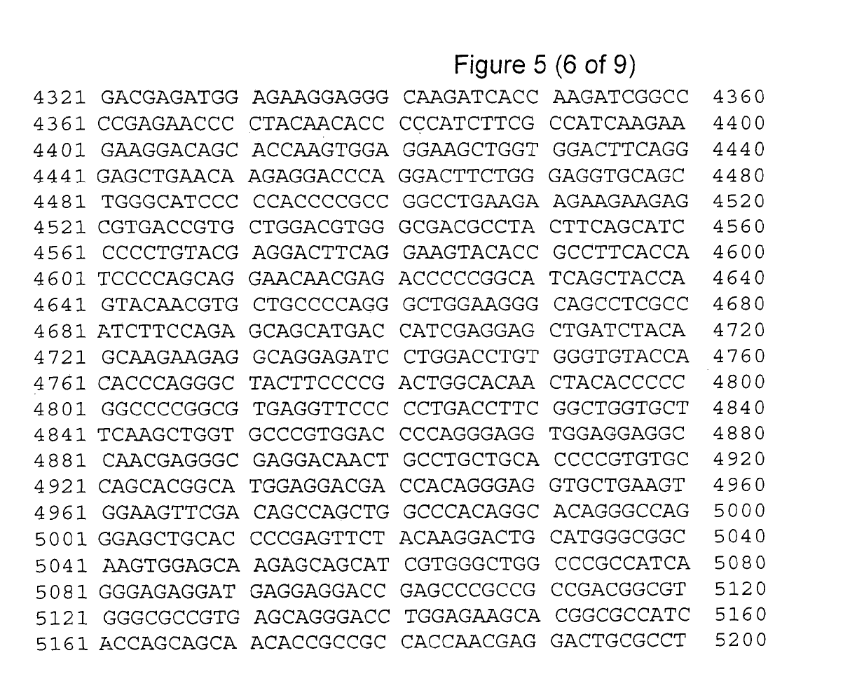

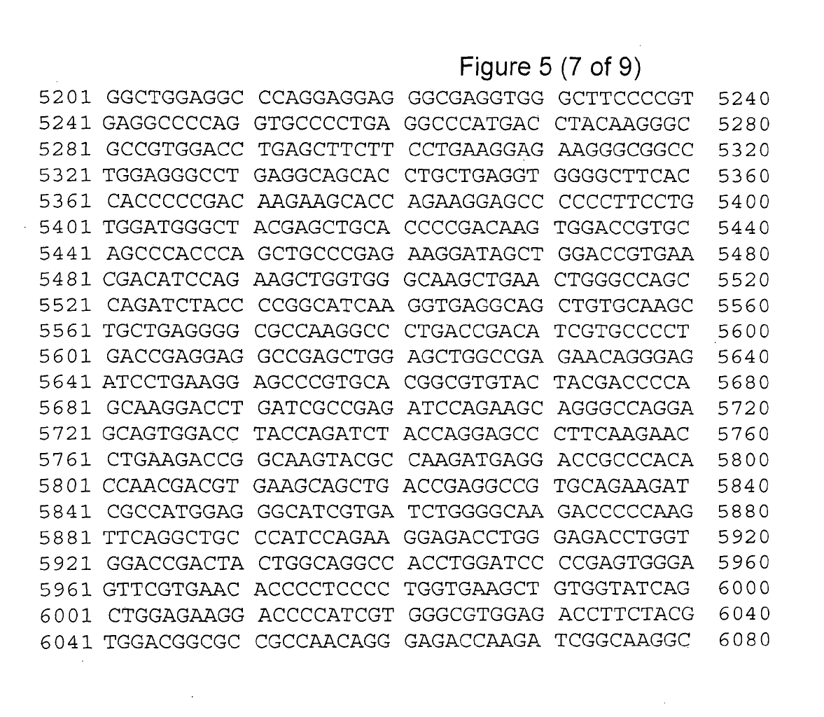

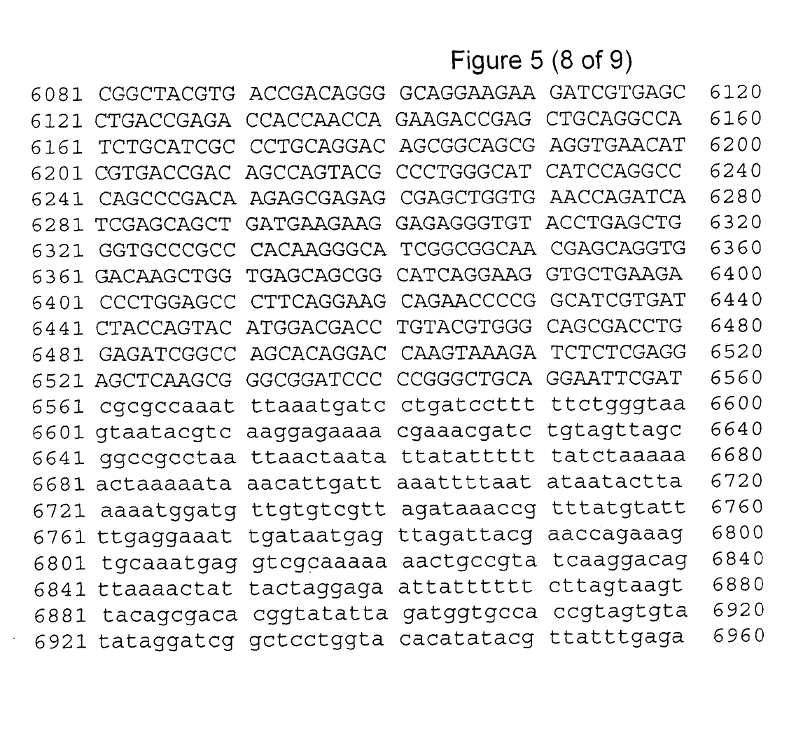

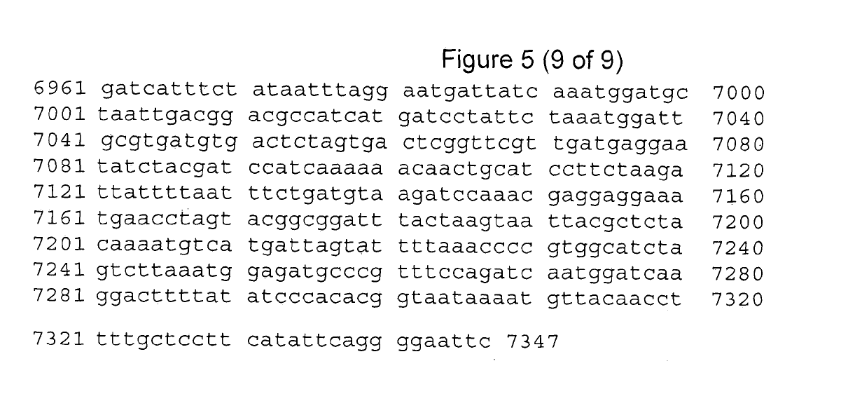

FIG. 5 shows the nucleotide sequence of MVA-C after homologous recombination.

FIG. 6 shows the nucleotide sequence of the 96ZM651-GPN plasmid vector.

FIG. 7 shows the amino acid sequences of the encoded GagPolNef (GPN) polypeptide.

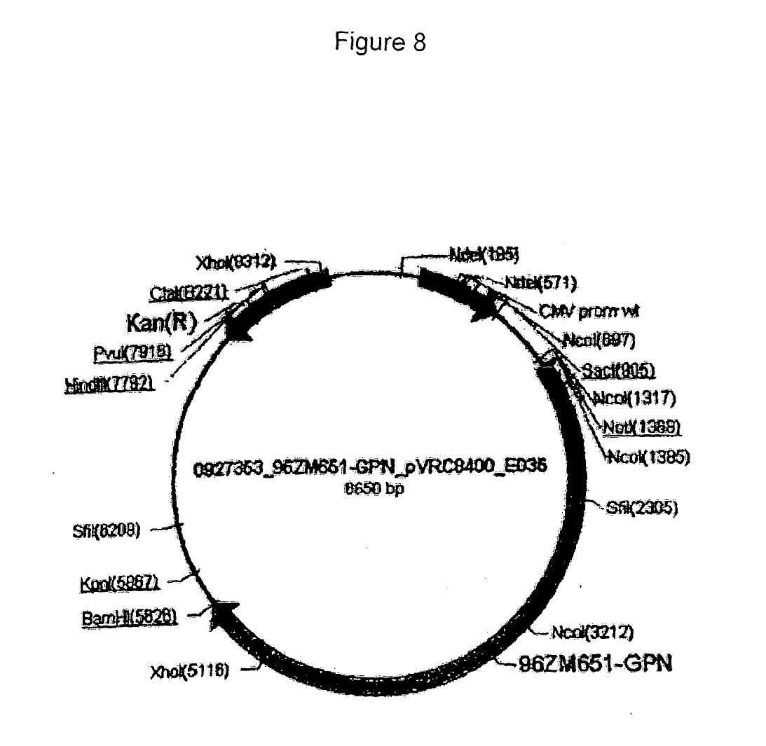

FIG. 8 is a plasmid map of 96ZM651-GPN.

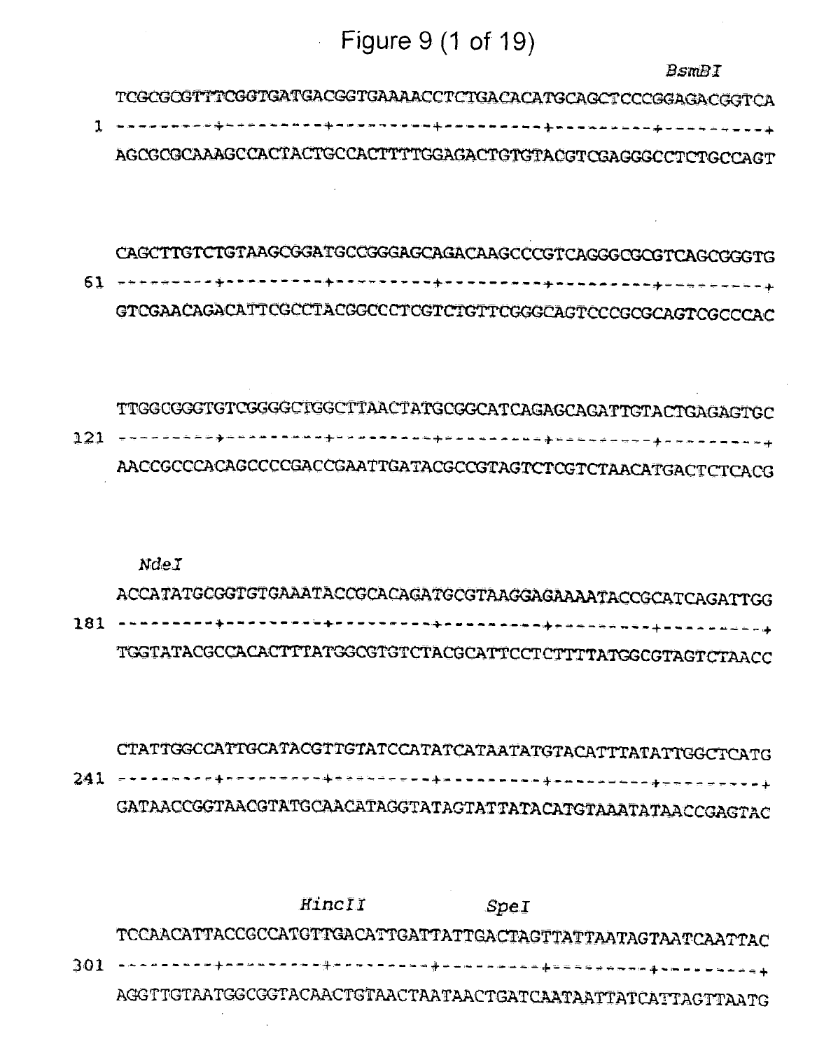

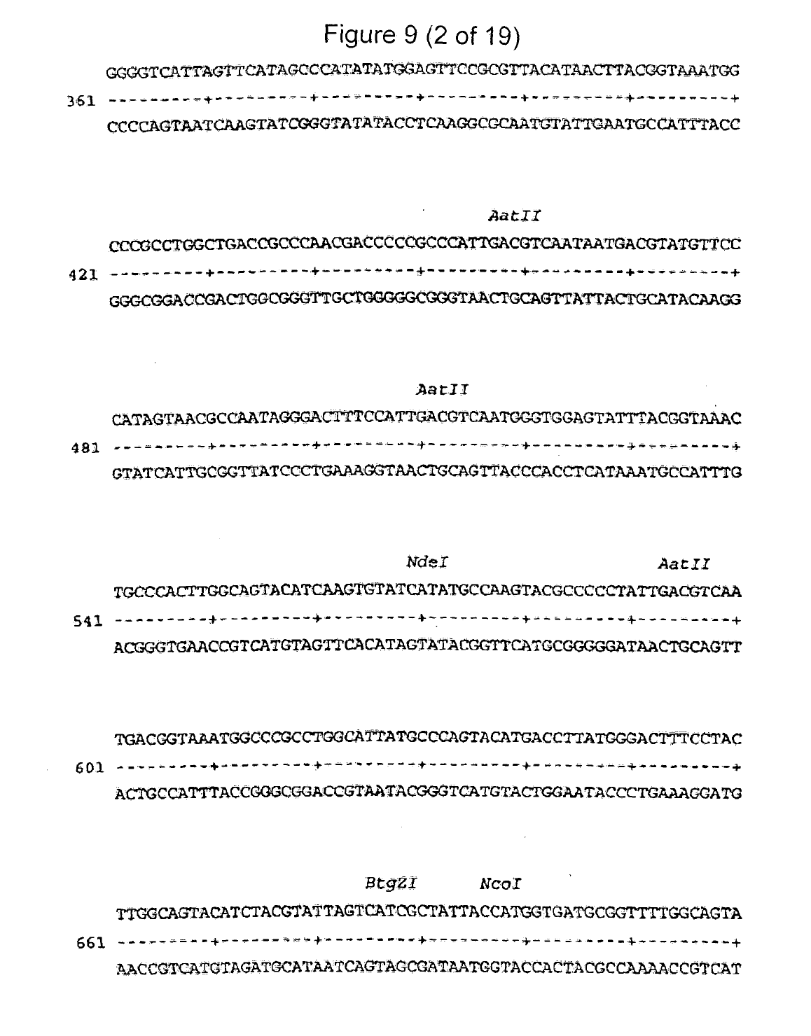

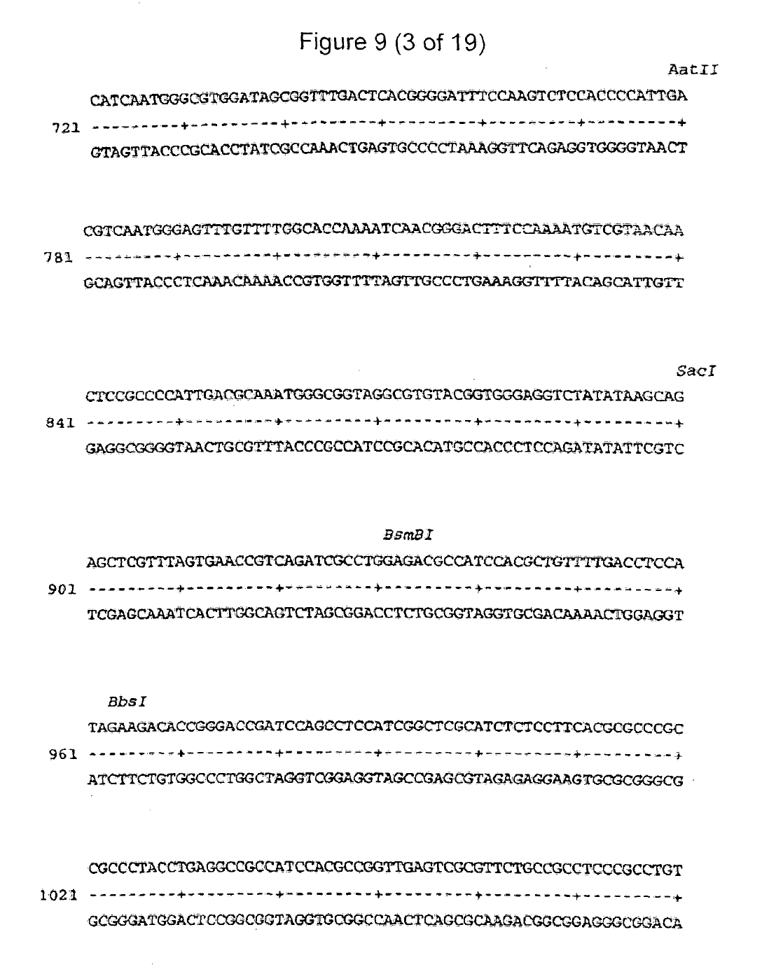

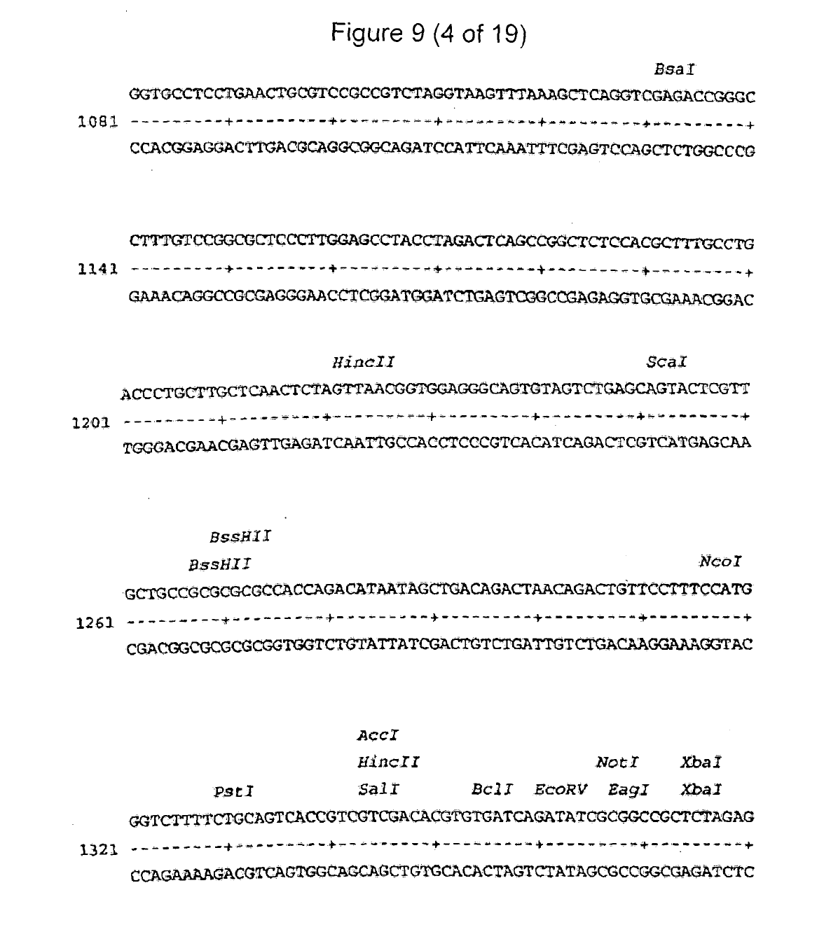

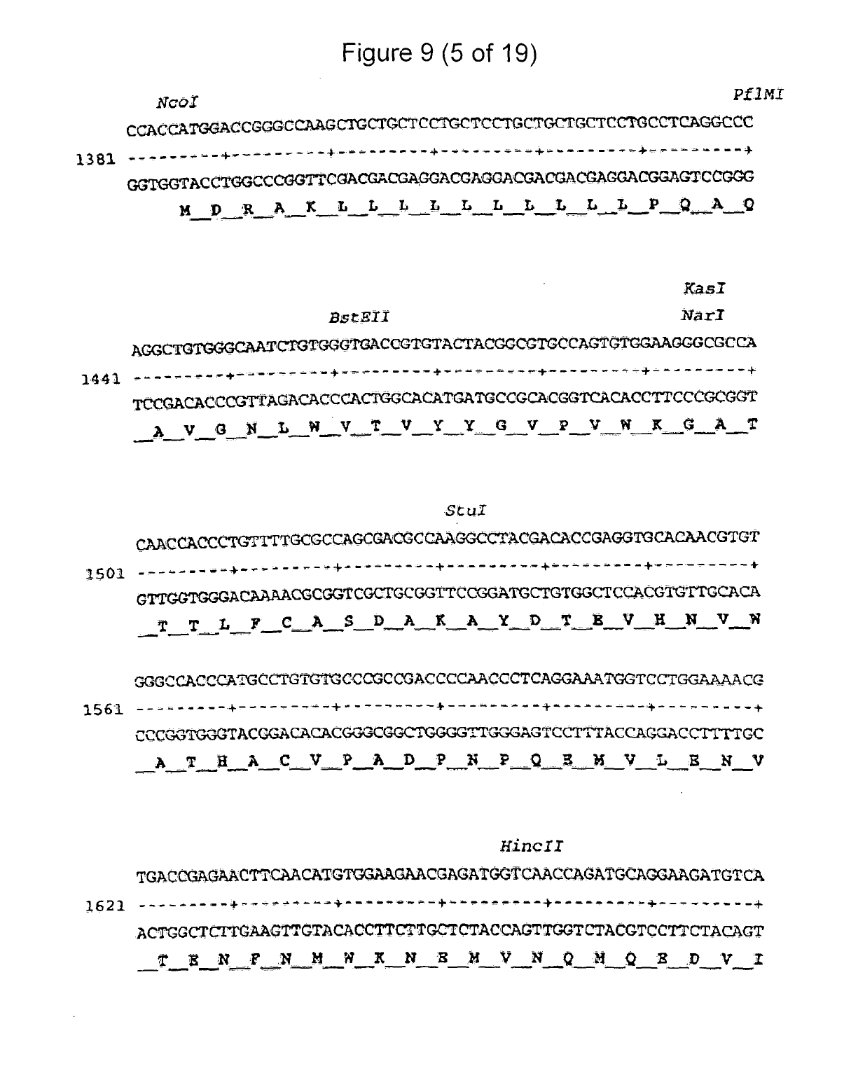

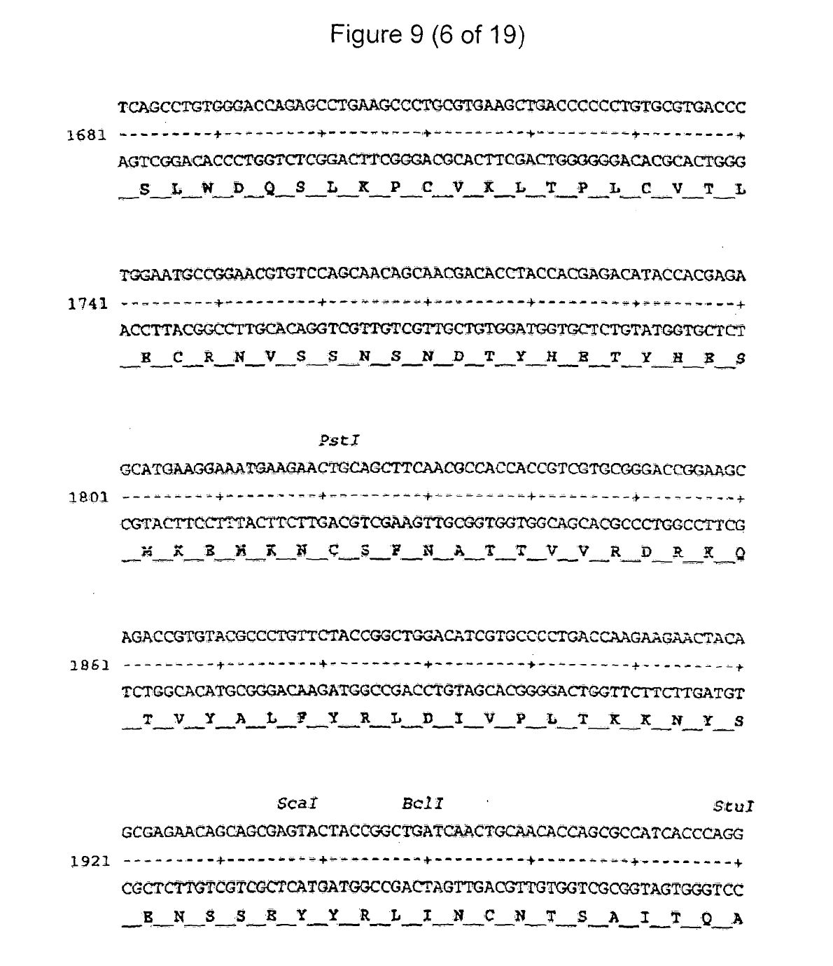

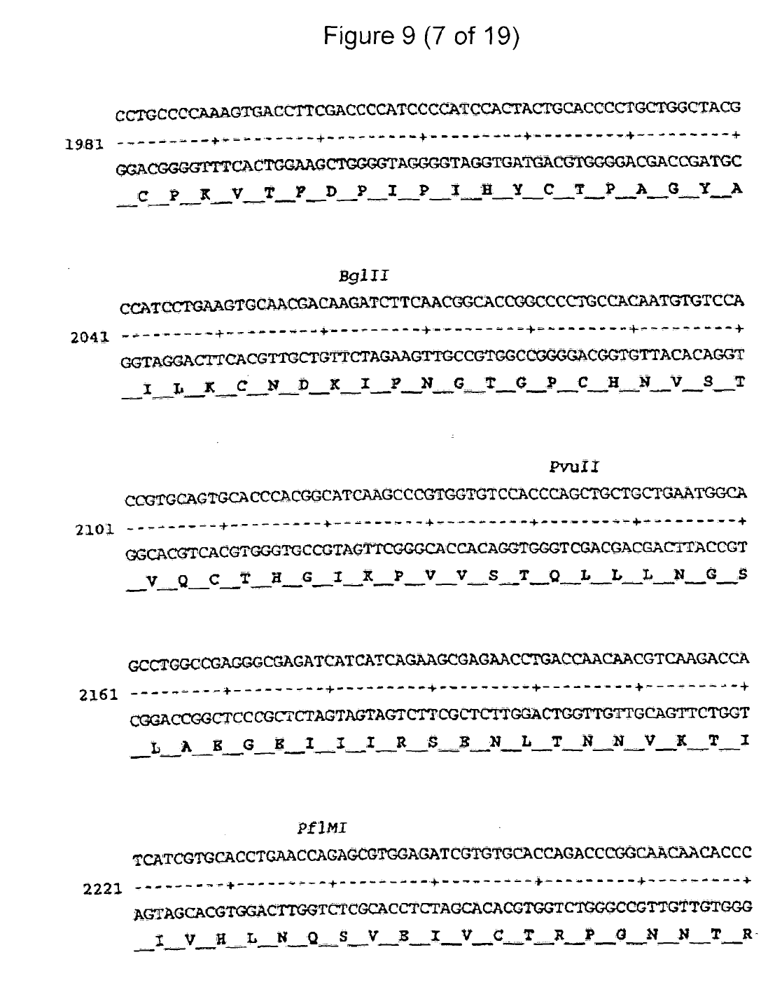

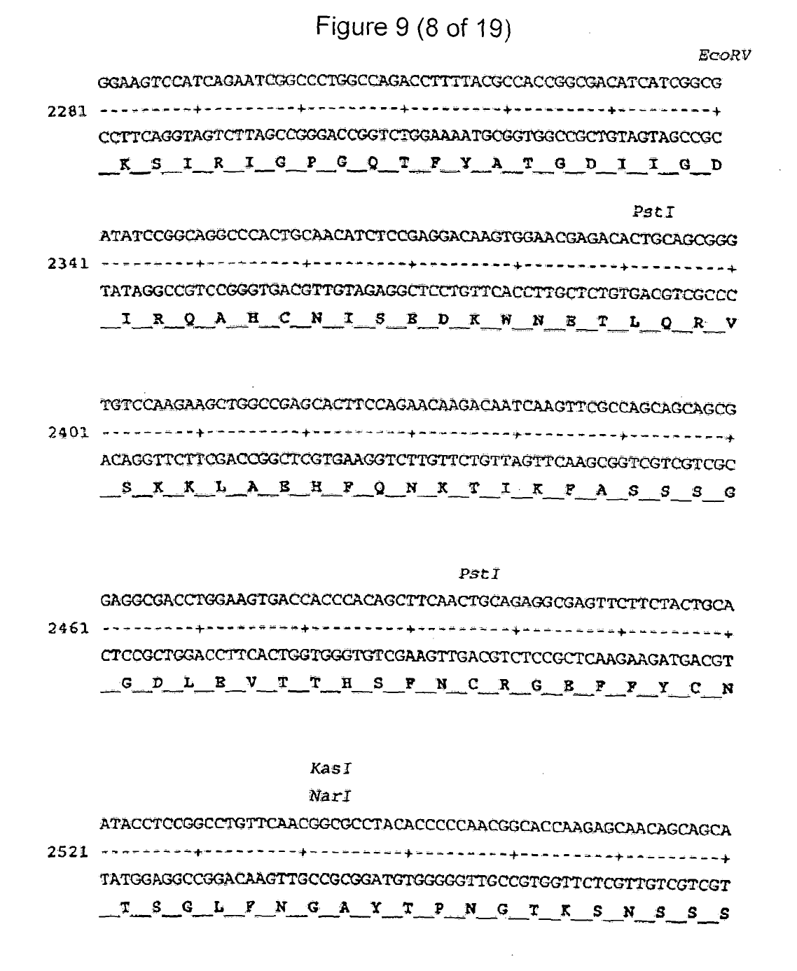

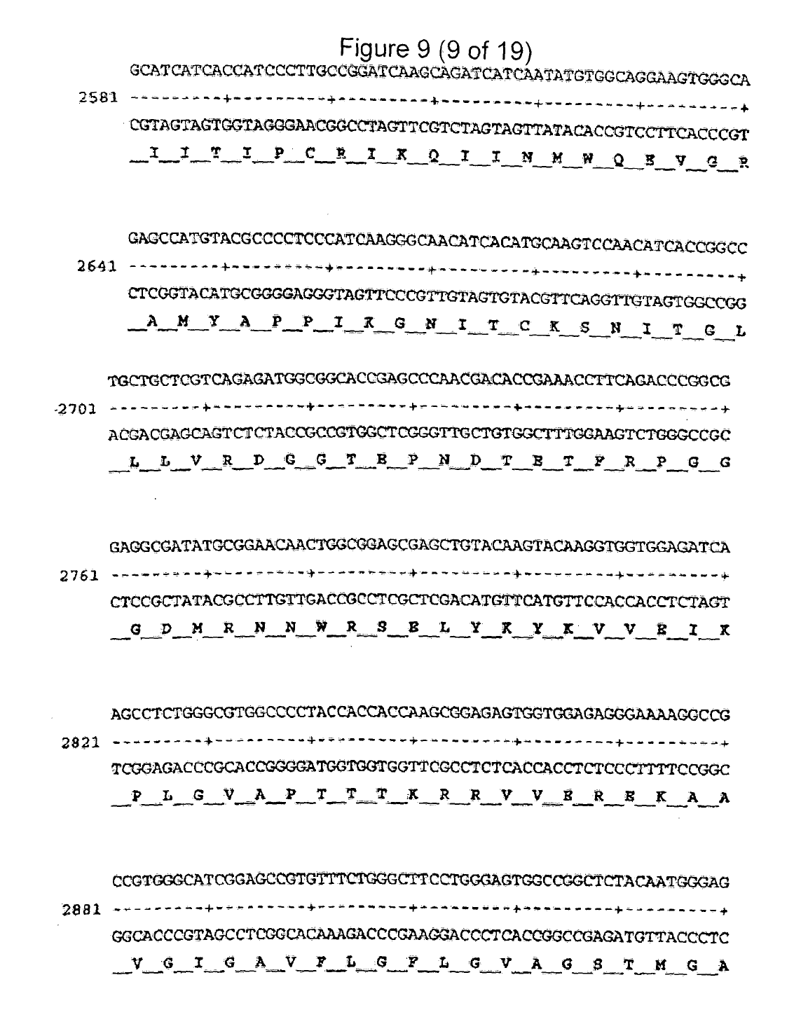

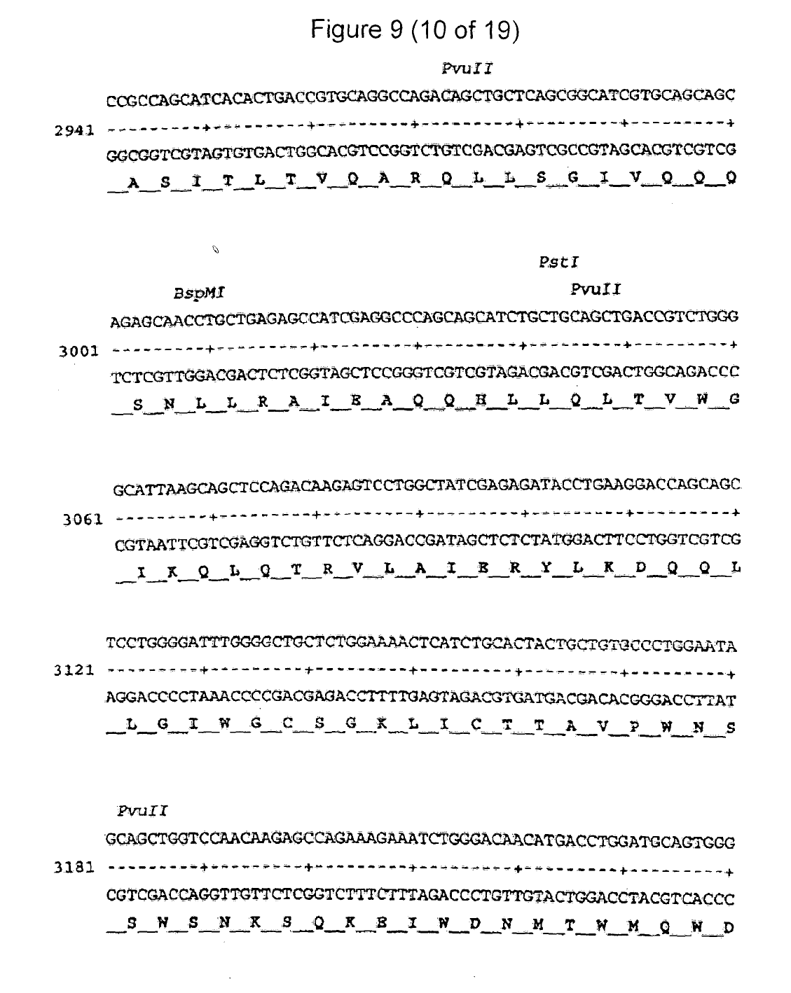

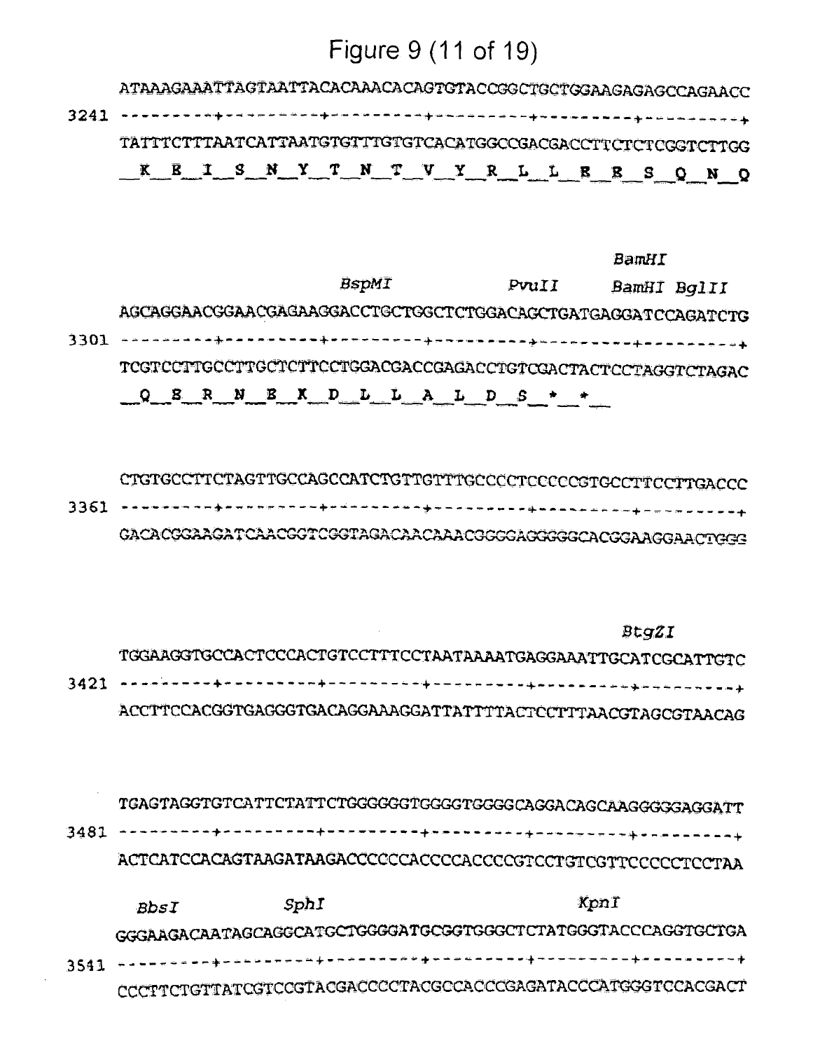

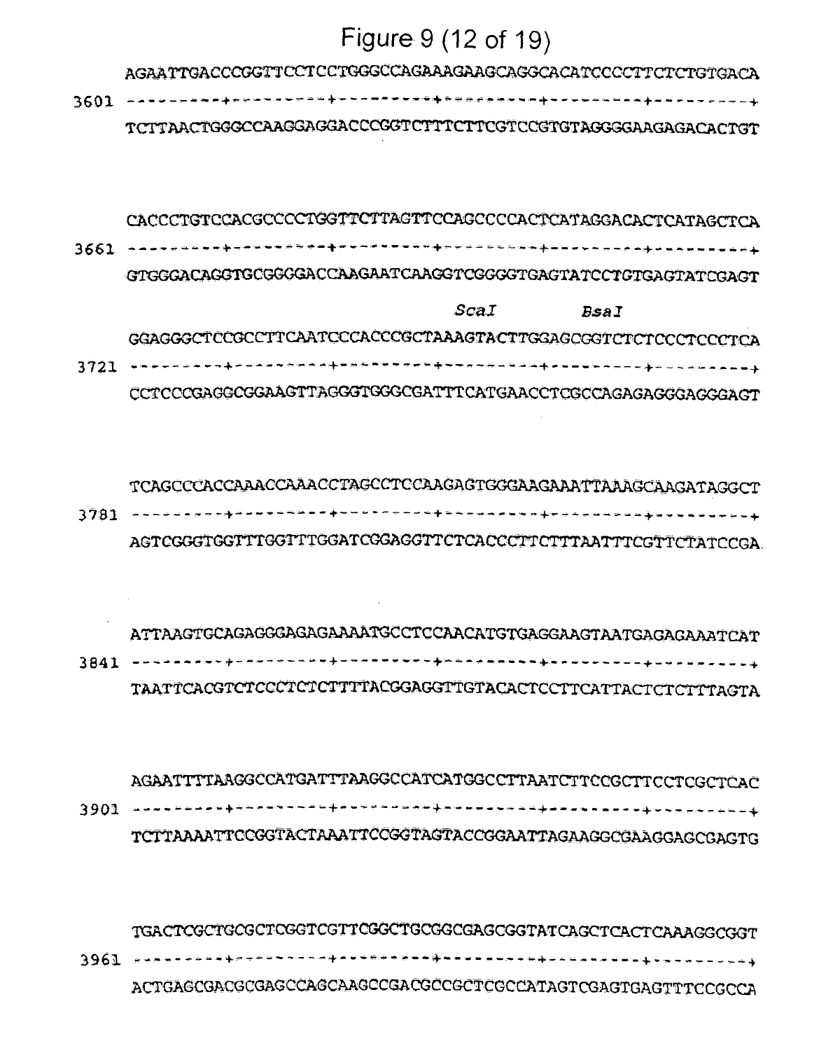

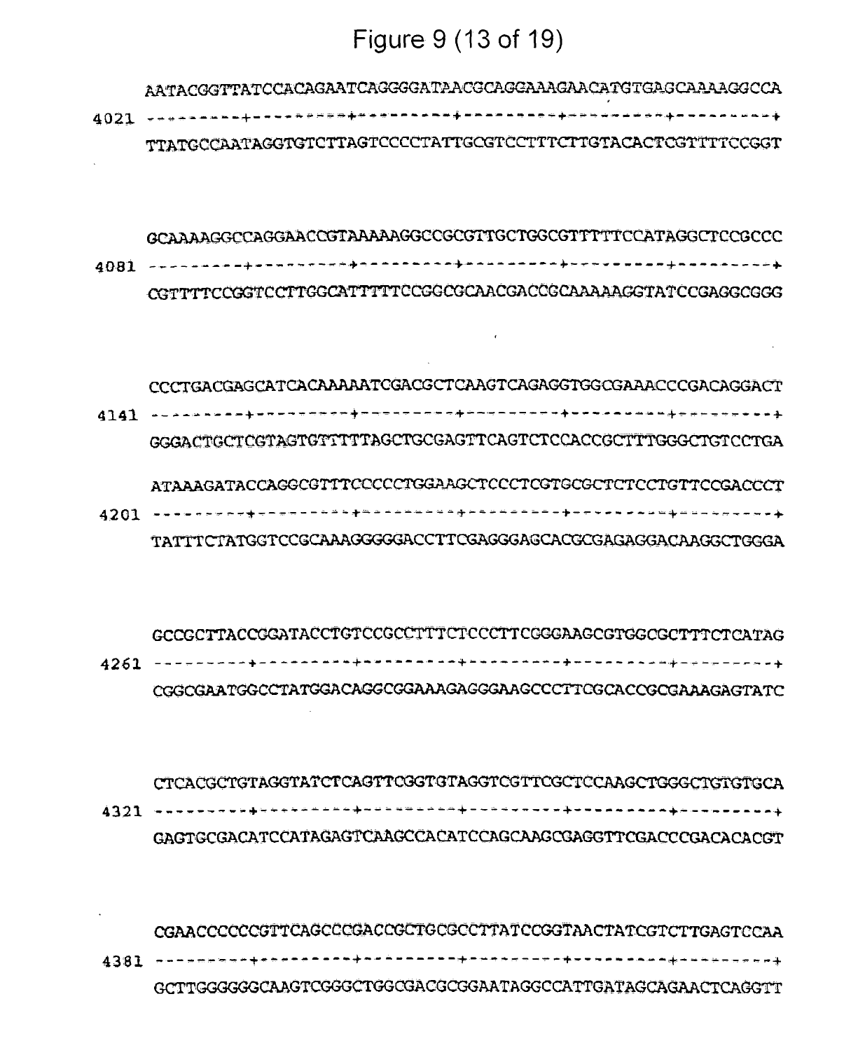

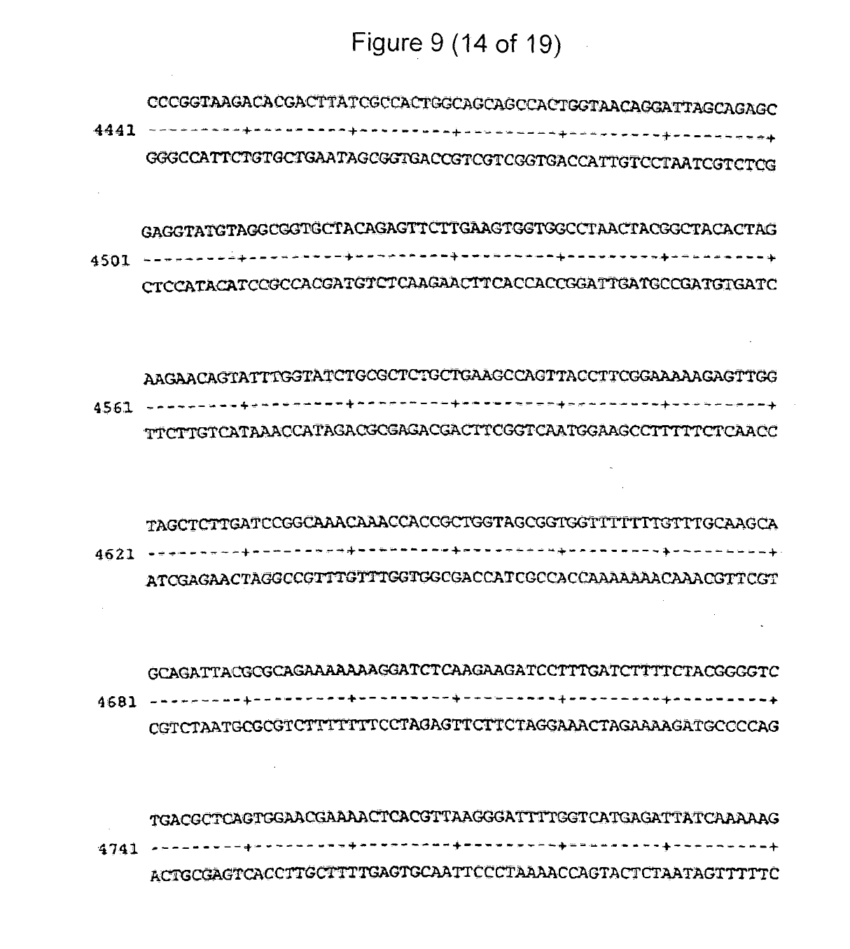

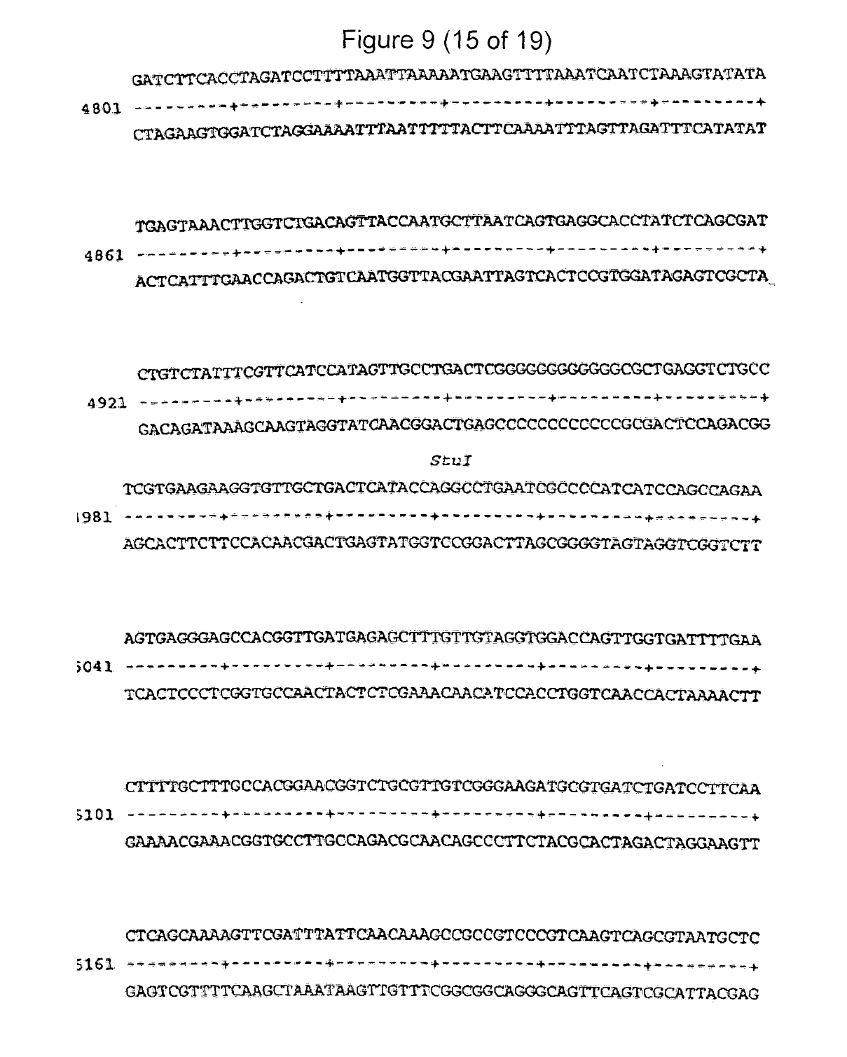

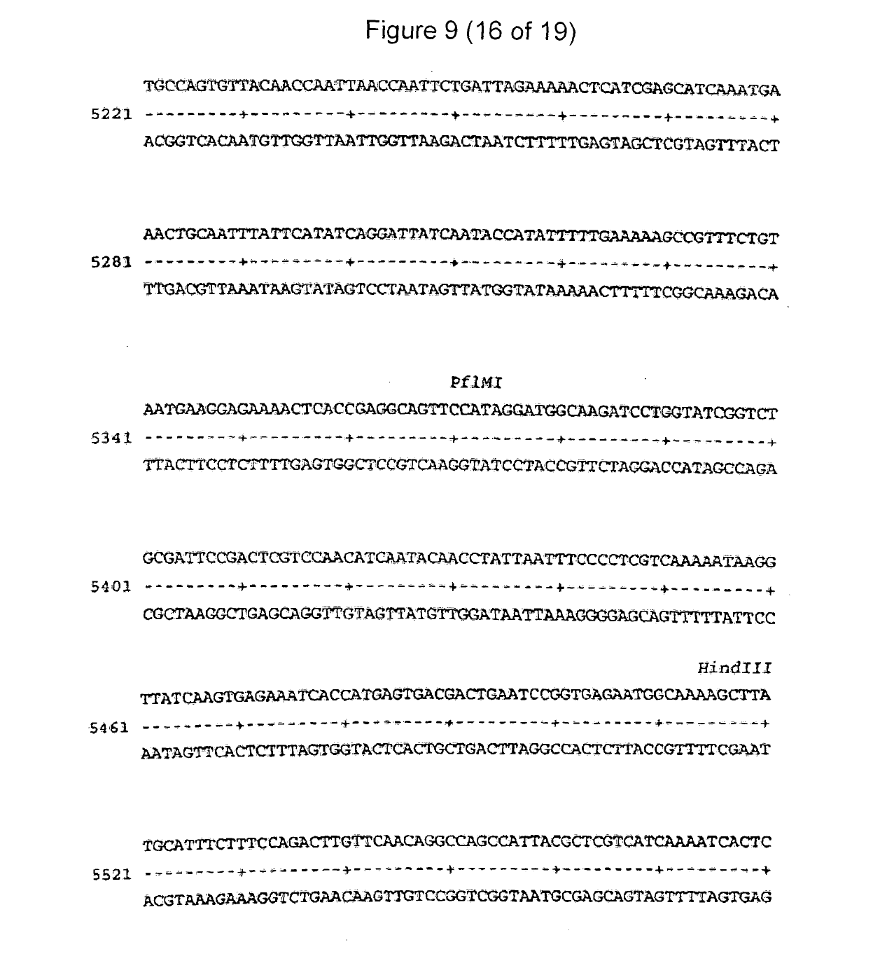

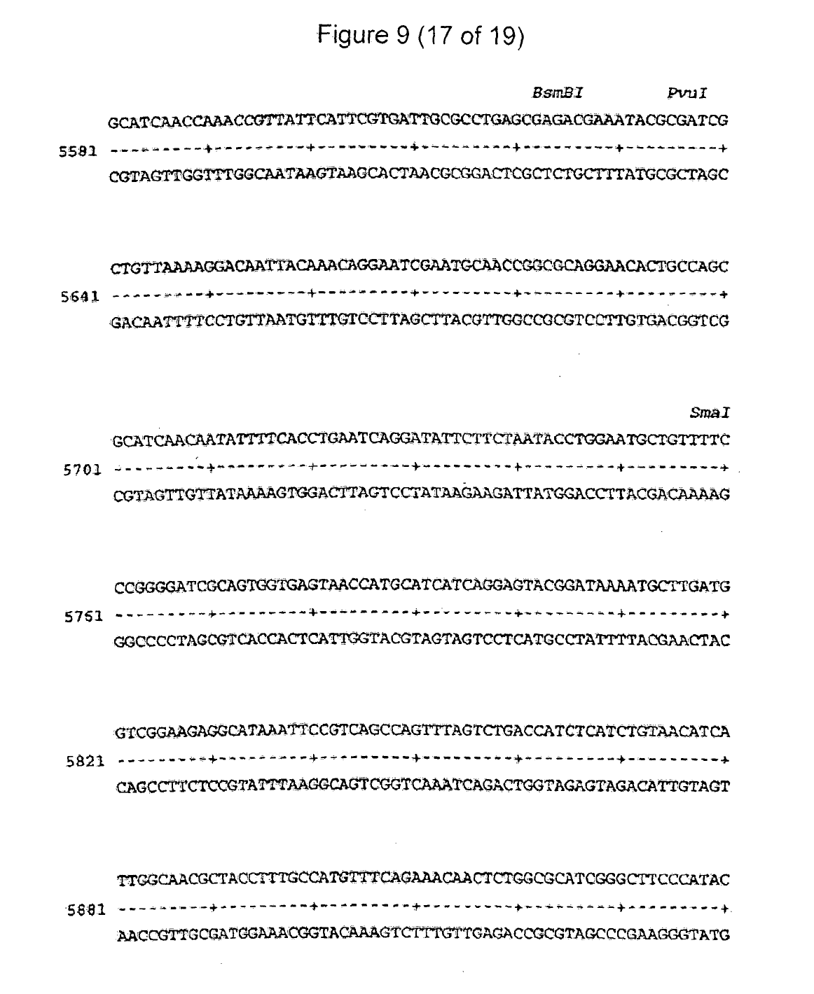

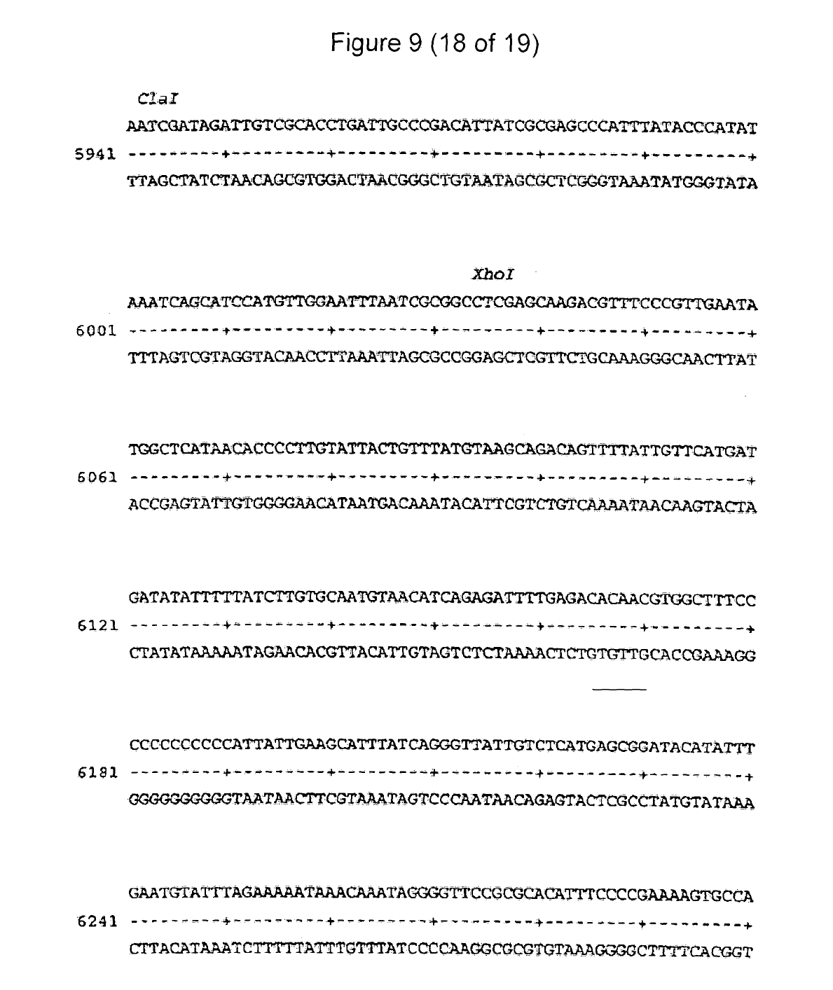

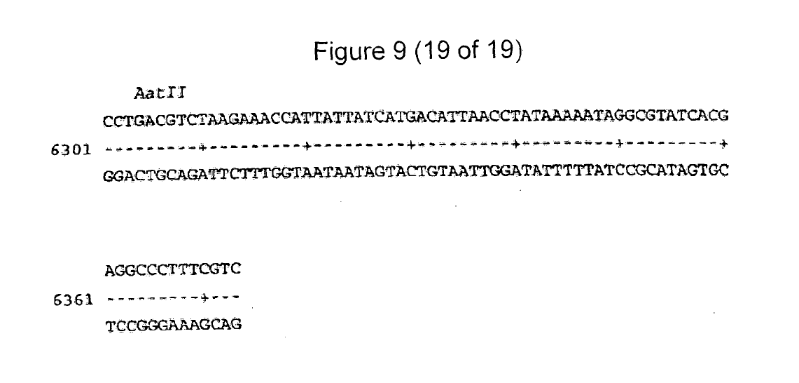

FIG. 9 shows the nucleotide sequence of the 97CN54-gp140 plasmid vector and the encoded amino acid sequence for gp140.

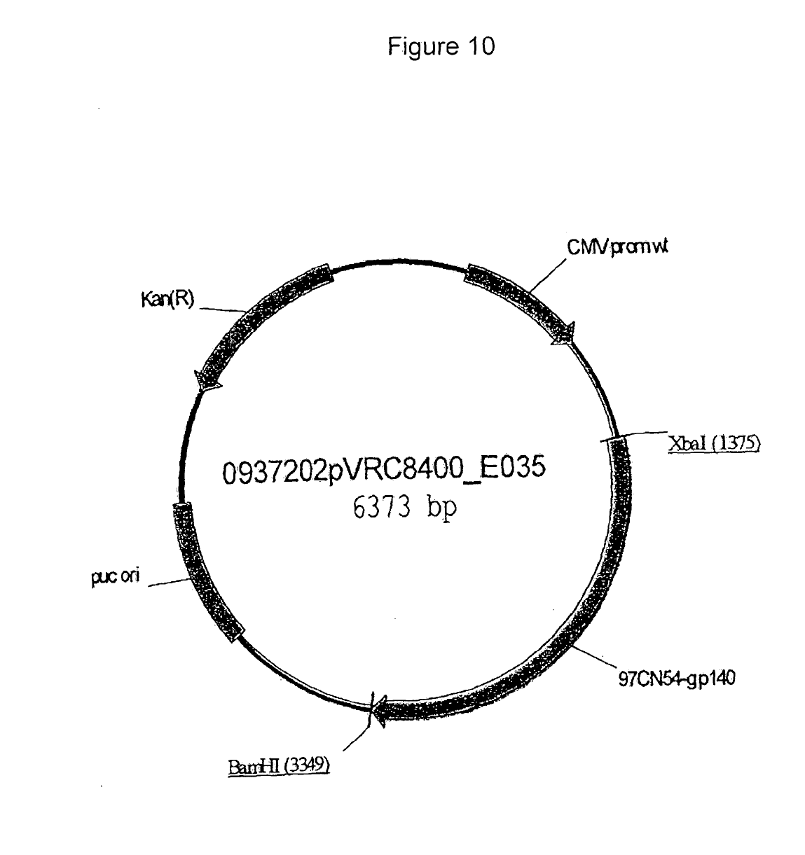

FIG. 10 is a plasmid map of 97CN54-gp140.

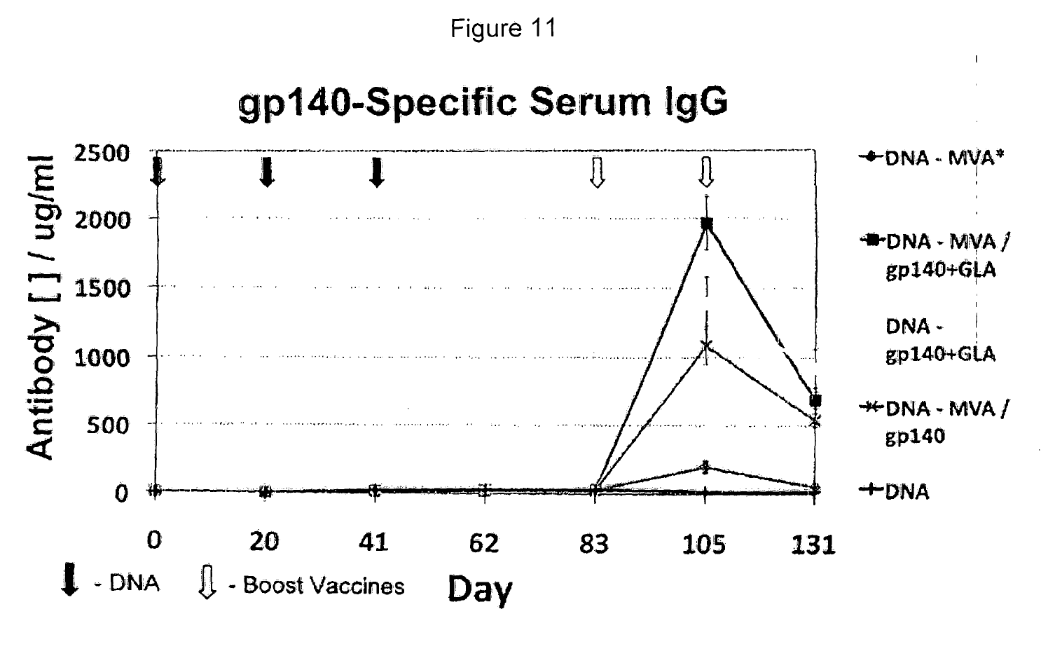

FIG. 11 shows gp140-Specific IgG responses in animals that had been primed with DNA.

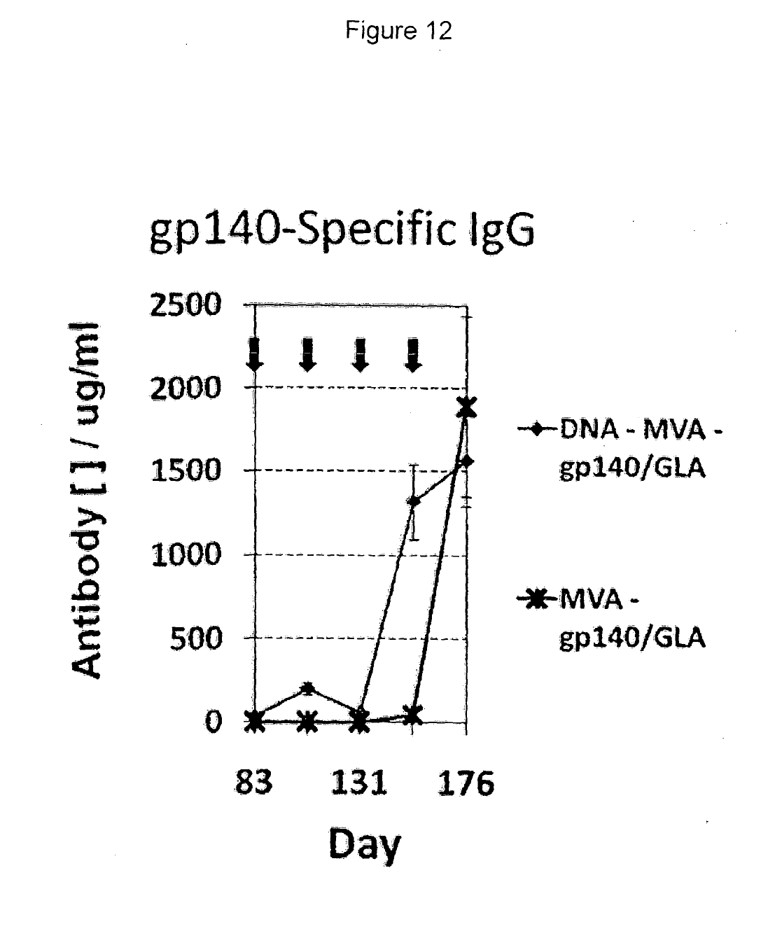

FIG. 12 shows gp140-Specific IgG responses in animals that were primed with DNA compared to DNA unprimed animals.

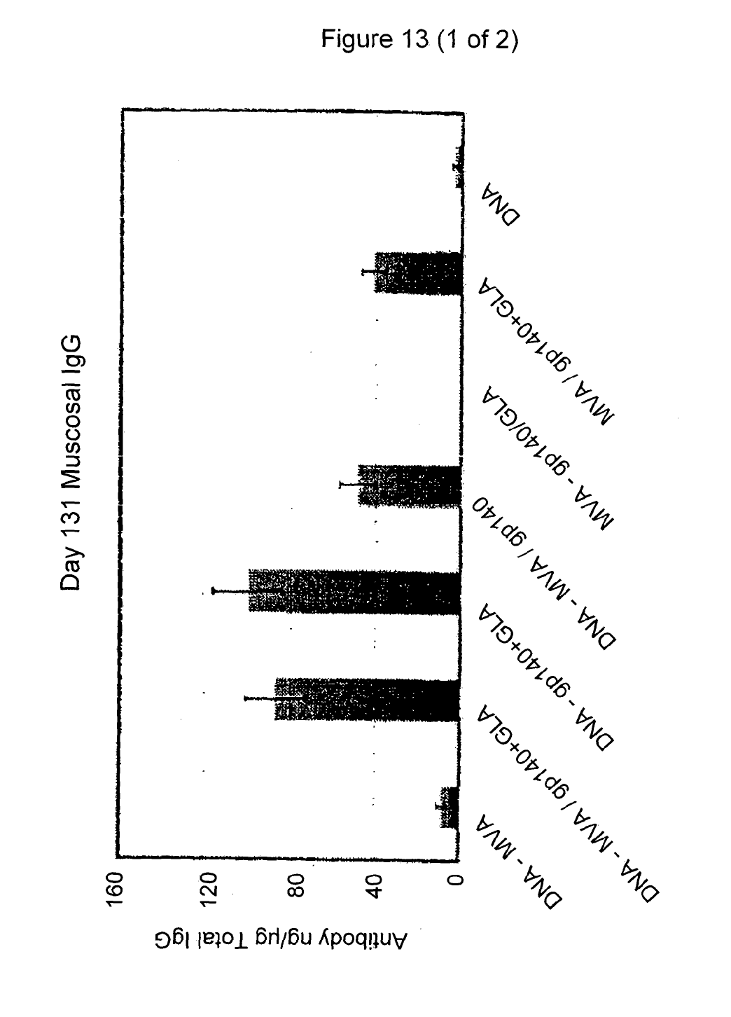

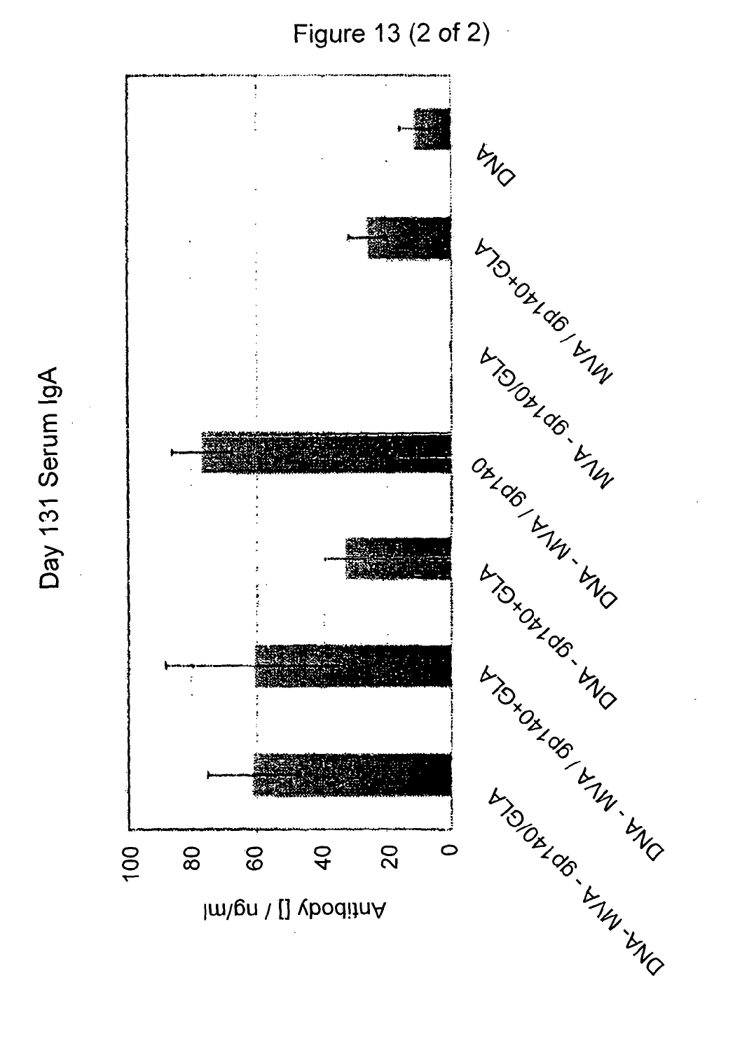

FIG. 13 shows antigen-specific serum IgA and mucosal IgG responses.

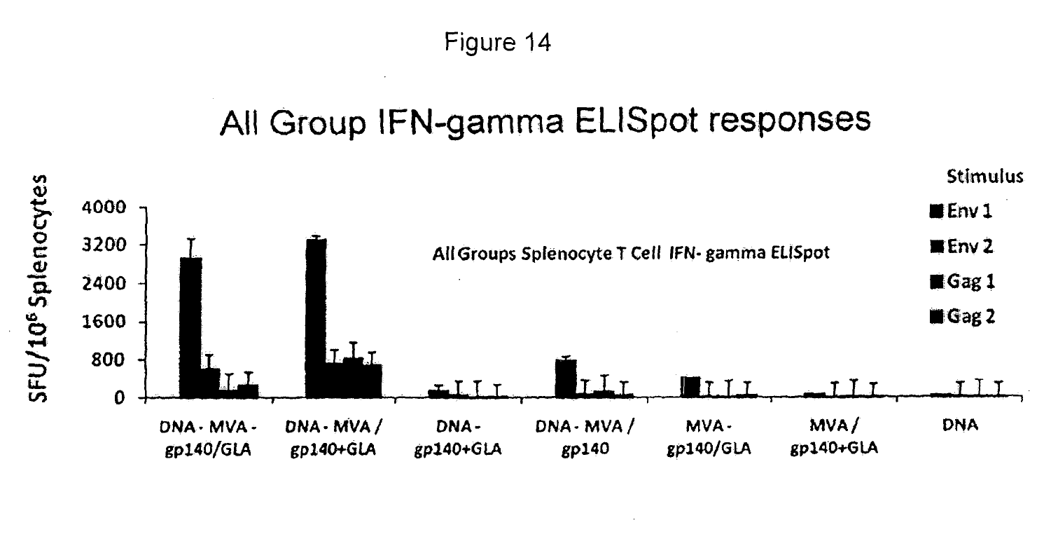

FIG. 14 shows splenocyte IFN-gamma responses to HIV CN54 Env and Gag peptide pools.

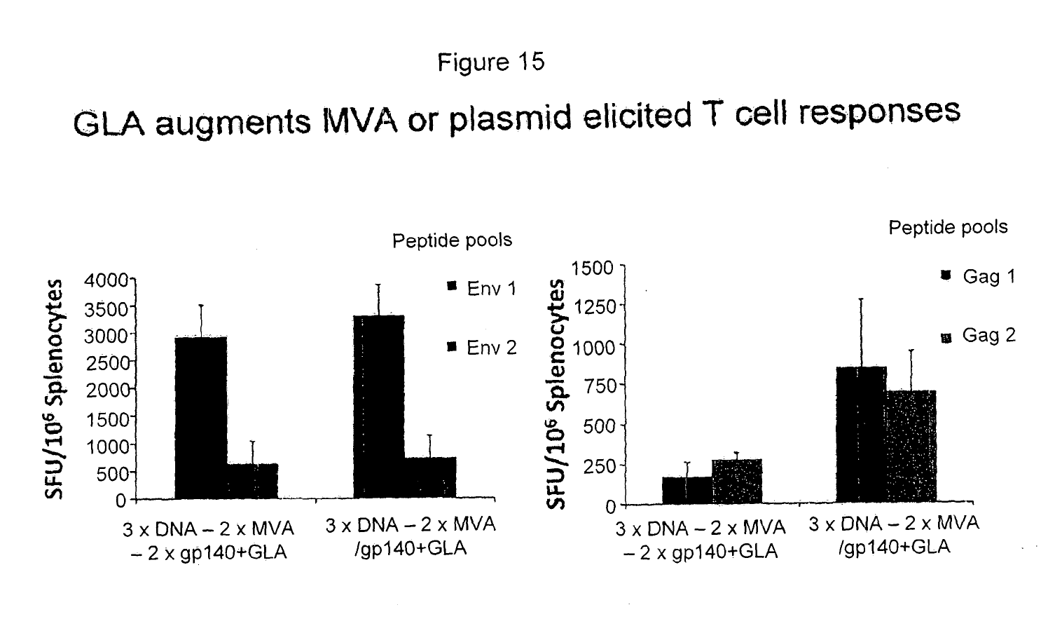

FIG. 15 shows significant augmentation of MVA elicited Gag responses in the presence of the GLA adjuvant.

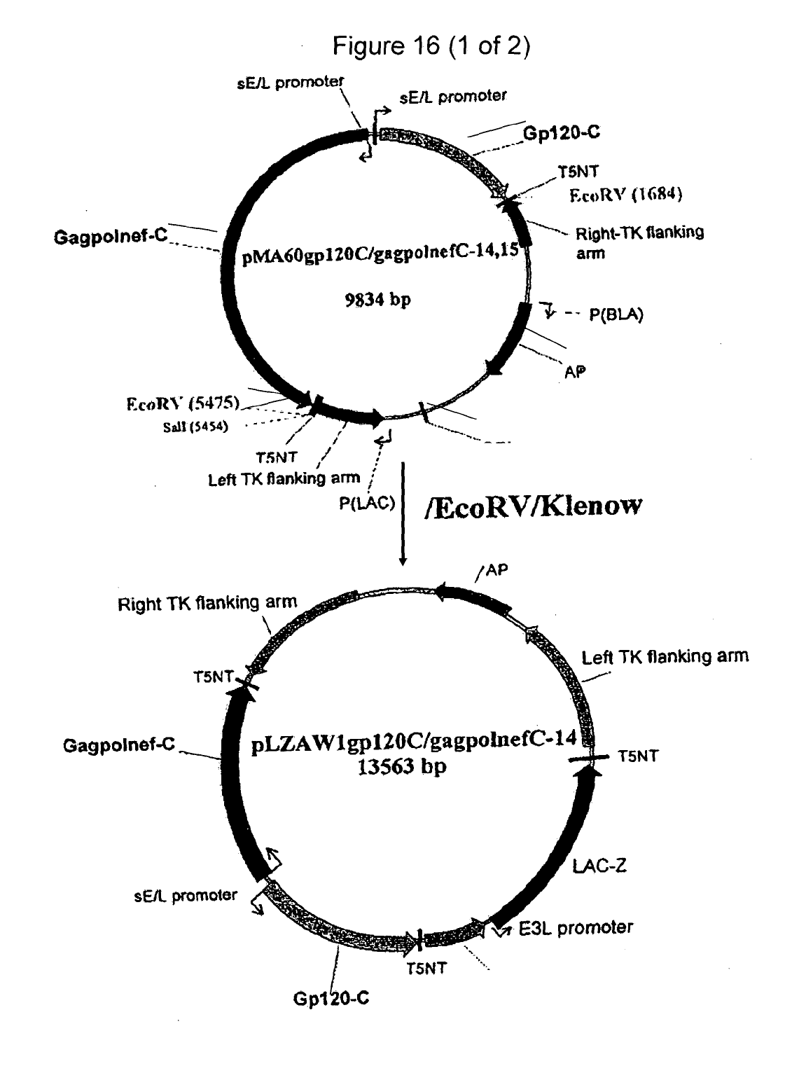

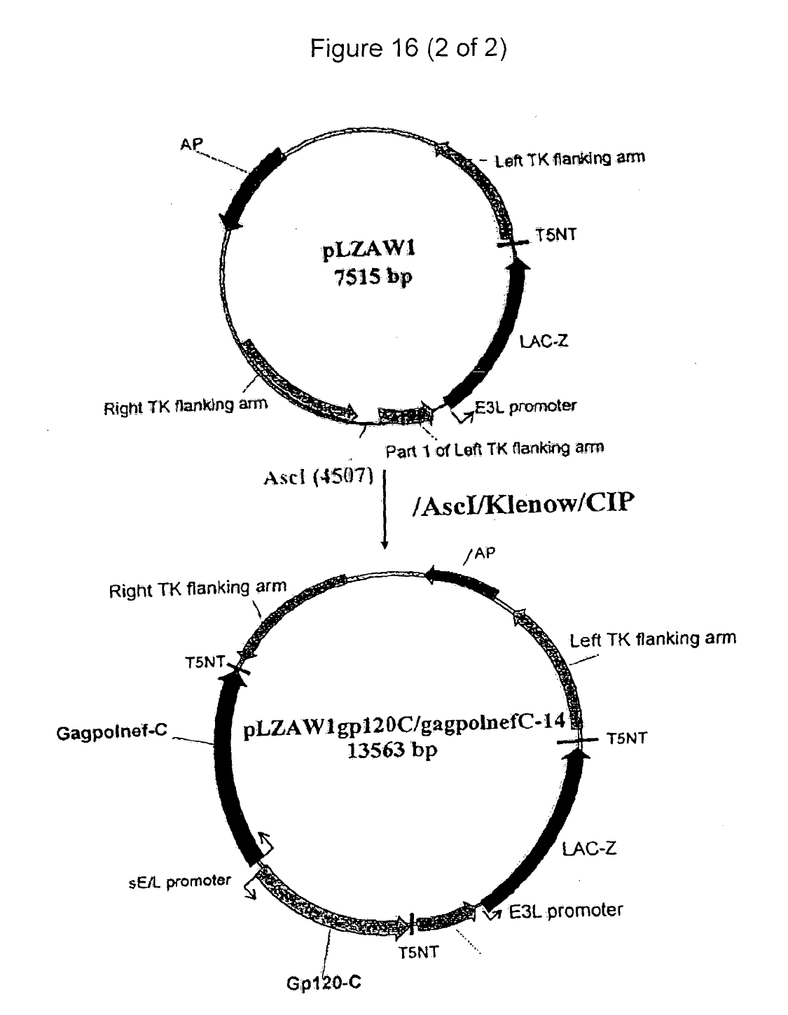

FIG. 16 is a scheme for the construction of the transfer vector pLZAW1gp120C/gagpolnefC-14.

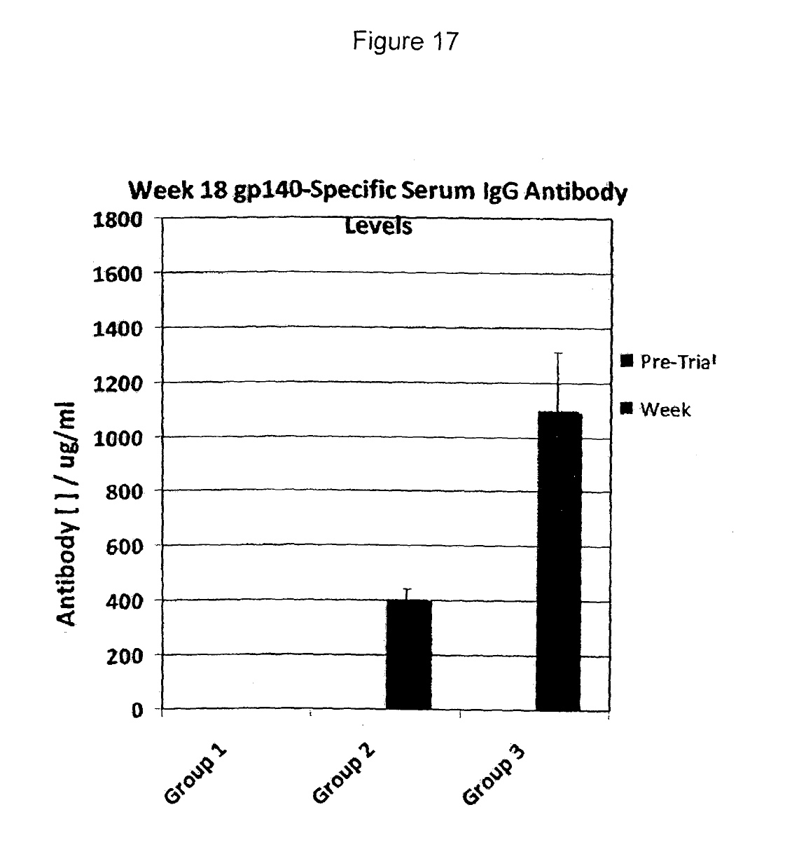

FIG. 17 shows week 18 gp140-specific serum IgG antibody levels in the rabbit trial described in Example 5 and mentioned in Example 4.

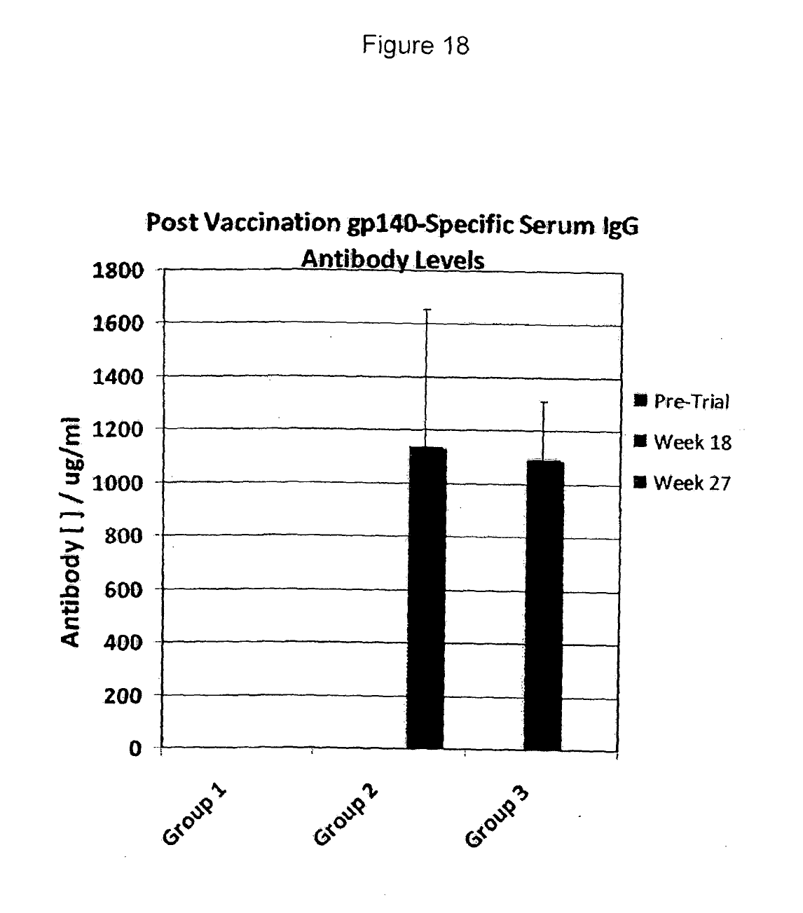

FIG. 18 shows post-vaccination gp140-specific serum IgG antibody levels in the rabbit trial described in Example 5 and mentioned in Example 4.

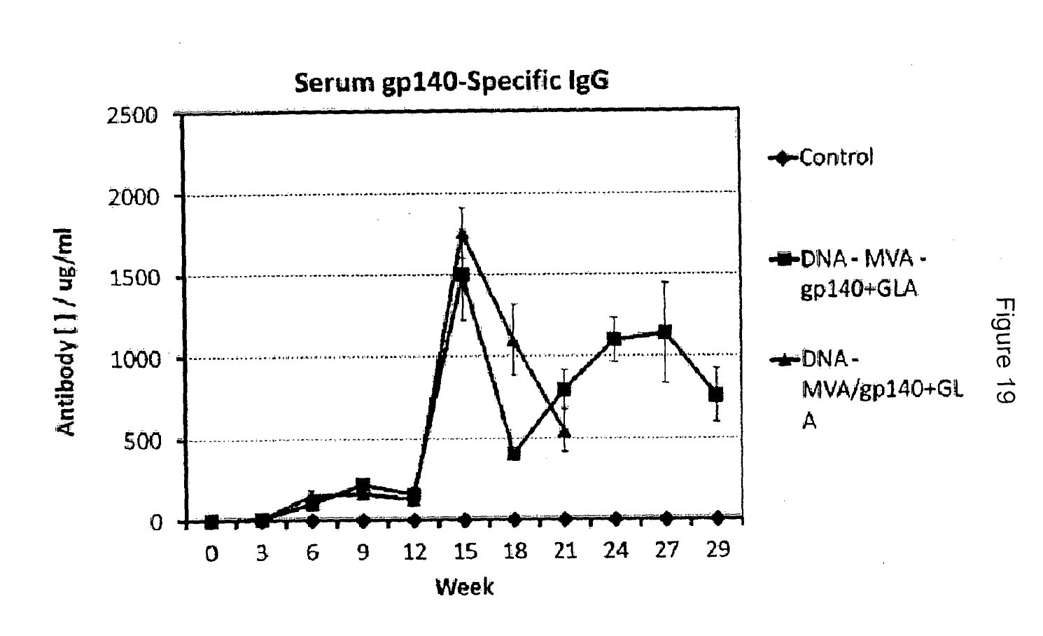

FIG. 19 also shows post-vaccination gp140-specific serum IgG antibody levels in the rabbit trial described in Example 4 and mentioned in Example 4.

### EXAMPLE 1

### Materials

### DNA-C ZM96GPN and DNA-C CN54ENV

The nucleotide sequence of the 96ZM651-GPN plasmid vector is shown in FIG. 6, and the encoded GagPolNef (GPN) amino acid sequence is shown in FIG. 7. FIG. 8 is a plasmid map of 96ZM651-GPN. FIG. 9 shows the nucleotide sequence of the 97CN54-gp140 plasmid vector and the encoded amino acid sequence for gp140. FIG. 10 is a plasmid map of 97CN54-gp140.

The design of the construct ZM96GPN include modifications to improve safety such as inactivation of the viral protease, use of a scrambled nef variant, and elimination of functions known to impair cellular metabolism such as down regulation of MHC or CD4. The products are expressed as a ˜160 kD GPN artificial polyprotein. One indication of its superior level of expression is that the expressed ZM96 polyprotein it is much easier to detect after the transient transfection of 293T cells. The env gene (CN54) is expressed in a separate plasmid designed to express a secreted GP140.

The 96ZM651-8 clone construct used as a template to built the DNA-C ZM96 vaccine is described in U.S. Pat. No. 6,492,110, issued Dec. 10, 2002, in U.S. Pat. No. 6,897,301, issued May 24, 2005, and in U.S. patent application Ser. No. 11/135,597, filed May 23, 2005 (UABRF Case No. U.S. Pat. No. 7,169,396), all of which are incorporated herein by reference.

TABLE 2
Reading frames of coding sequences of GPN fusion construct
of HIV-1 strain 96ZM651

| | GPN | | Genomic position | | |
|---|---|---|---|---|---|
| ORF | Start | End | Start | End | Modifications |
| gag | 1 | 1482 | 137 | 1618 | G2A^{a} |
| 5′ pol (ΔRT) | 1483 | 2430 | 1426 | 2373 | D77N^{b} |
| Scrambled nef | 2431 | 2760 | 8461 | 8790 | |
| 3′ nef | | | | | |
| 5′ nef | 2761 | 3060 | 8173 | 8472 | |
| 3′ pol (ΔIN) | 3061 | 4125 | 2494 | 3558 | |
| RT active site | 4126 | 4233 | 2374 | 2481 | |

^{a}Knock out of myristylation site, ^{b}inactivation of protease active site

Toxicology and safety of DNA-C: DNA plasmids containing the same inserts have already been injected into healthy human volunteers at the same concentrations. The plasmids have been through a further round of codon optimisation and have a different backbone to improve immunogenicity but we have no reason to suspect that these modifications will impact on safety.

### MVA-C GPN/ENV

The MVA-C vaccine contains the same inserts as used previously in NYVAC-C (gag, poi, nef and env), and is described in Gomez et al (2007) Vaccine 25, 1969-1992, incorporated herein by reference. The MVA-C immunogen is also described in PCT/ES2006/070114 (WO 2007/012691), incorporated herein by reference.

Construction of Vector Plasmid pLZAW1gp120C/Gagpolnef-C-14

The vector plasmid pLZAW1gp120C/gagpolnef-C-14 was constructed for engineering the recombinant MVA virus expressing the HIV-1 genes from clade C (CN54), gp120 and gagpolnef. The plasmid is a pUC derivative designed for a blue/white plaque screening. It contains TK left and right flanking sequences, a short TK left arm repeat, an E3L promoter driven β-gal expression cassette and the AP gen. Between the two flanking sequences, there are two synthetic early/late (E/L) promoters in a back-to-back orientation individually driving a codon optimized gp120 and gagpolnef genes of HIV-1 clade C. The positions of all the components included in the plasmid are described in Table 3.

TABLE 3

| | | |
|---|---|---|
| Left TK flanking sequence | 410-908 | complementary |
| T5NT for β-gal | 929-935 | complementary |
| β-gal | ATG-TAA (936-4079) | complementary |
| E3L promoter for β-gal | 4080-4140 | complementary |
| Part 1 of Left TK flanking | 4151-4498 | complementary |
| sequence | | |
| T5NT for gp120 | 4607-4614 | complementary |
| Gp120 | ATG-TGA (4643-6139) | complementary |
| E/L promoter for gp120 | 6149-6187 | complementary |
| E/L promoter for gagpolnef | 6202-6240 | |
| Gagpolnef | ATG-TAA (6250-10503) | |
| T5NT for gagpolnef | 10584-10590 | |
| Right TK flanking sequence | 10652-11343 | complementary |
| AP | ATG-TAA (12514-13374) | complementary |

The complete clade C/B′ HIV-1 97CN54 coding sequence and codon optimized genes is disclosed in EP1240333B1, incorporated herein by reference A 6.047 kbp DNA fragment containing the two synthetic early/late (E/L) promoters in a back-to-back orientation individually driving a codon optimized gp120 and gagpolnef genes of HIV-1 clade C (CN54) was excised with EcoRV from plasmid MA60gp120C/gagpolnefC-14,15, modified by incubation with Klenow DNA polymerase to generate blunt ends, and cloned into pLZAW1 vector (previously digested with restriction endonuclease Ascl, modified by incubation with Klenow, and dephosphorylated by incubation with Alkaline Phosphatase, Calf Intestinal (CIP)) generating the plasmid transfer vector pLZAW1gp120C/gagpolnef-C-14 (13564 pb) (FIG. 16). The plasmid pLZAW1gp120C/gagpolnef-C-14 directs the insertion of the foreign genes into the TK locus of MVA genome. After the desired recombinant virus has been isolated by screening for expression of β-galactosidase activity further propagation of the recombinant virus leads to the self-deletion of β-gal by homologous recombination between the TK left arm and the short TK left arm repeat that are flanking the marker.

### Construction and Characterization of Recombinant Virus MVA-C

Primary chickEN embryo fibroblast cells (CEF) from 11-day old SPF eggs (INTERVET) were infected with MVA (MVA-F6, passage 585, provided by Gerd Sutter) at a multiplicity of 0.05 PFU/cell and then transfected with 10 μg DNA of plasmid pLZAW1gp120C/gagpolnef-C-14 using lipofectamine reagent according to the manufacture instructions (Invitrogen, Cat. 18324-012, lot 1198865). After 72 h post infection the cells were harvested, sonicated and used for recombinant virus screening.

Recombinant MVA viruses containing the gp120C/gagpolnef-C genes and transiently co-expressing the β-gal marker gene (MVA-C(X-gal+)) were selected by consecutive rounds of plaque purification in CEF cells stained with 5-bromo-4-chloro-3-indolyl β-galactoside (300 μg/mL). In the following, recombinant MVA viruses containing the gp120C/gagpolnef-C genes and having deleted the p-gal marker gene (MVA-C(X-gal-)) were isolated by two additional consecutive rounds of plaque purification screening for non-staining viral foci in CEF cells in the presence of 5-bromo-4-chloro-3-indolyl β-galactoside (300 μg/mL). In each round of purification the isolated plaques were expanded in CEF cells for 3 days, and the crude virus obtained were used for the next plaque purification round. In the first round screening 10 X-gal+ plaques designated as MVA-C (−1 to 10) were isolated. Only MVA-C-1 that efficiently expresses the gp120C and gagpolnef-C antigens was amplified and used for the next plaque purification round. In the second passage were isolated 20 X-gal+ plaques designated as MVA-C (−1.1 to 1.20) all of them expressing both proteins. The MVA-C-1.7 and MVA-C-1.13 were amplified and used for the next plaque purification round. In the third passage were isolated 11 X-gal+ plaques (designated as MVA-C-1.7.8 to 1.7.12 and MVA-C-1.13.7 to 1.13.12) and 13 X-gal-plaques (designated as MVA-C-1.7.1 to 1.7.7 and MVA-C-1.13.1 to 1.13.6). The MVA-C-1.7.1 and MVA-C-1.13.5 were amplified and used for the next plaque purification round. In the fourth passage were isolated 24 X-gal-plaques designated as MVA-C-1.7.1.1 to 1.7.1.12 and MVA-C-1.13.5.1 to 1.13.5.12. The recombinants designated as MVA-C-1.7.1.2 and MVA-C-1.13.5.7 were used to prepare the P2 stocks. The P2 stock of MVA-C-1.7.1.2 with titer 0.7×108 PFU/mL was send for GMP production. A P3 stock (purified from CEF cells infected at moi 0.05 by two 36% sucrose cushion) with titer 4.25×108 PFU/mL was prepared.

The nucleotide sequence of pLZAW1gp120C/gagpolnefC-14 transfer vector is shown in FIG. 4. The nucleotide sequence of the MVA-C vector after homologous recombination is shown in FIG. 5.

Toxicology and Safety of MVA-C:

There were no significant safety concerns during EV01-3 using NYVAC-C containing similar inserts. We do not anticipate any safety concerns since the MVA vector is widely used and has been injected into many thousands of volunteers forming the basis of several programmes of vaccine development (HIVIS/Walter Reed, HVTN/SAAVI, Oxford) in a variety of disease contexts and settings. MVA-based vaccines have also been shown to be safe and well tolerated by those who are immunosuppressed.

rGP140 and GLA-AF

GLA-AF is an aqueous adjuvant formulation containing glucopyranosyl lipid A, a completely synthetic monophosphoryl lipid A (MPL®)-like molecule which acts as a toll-like receptor 4 (TLR4) agonist (Coler R N, Bertholet S, Moutaftsi M, Guderian J A, Windish H P, et al (2011) Development and Characterization of Synthetic Glucopyranosyl Lipid Adjuvant System as a Vaccine Adjuvant. PLoS ONE 6(1): e16333. doi:10.1371/journal.pone.0016333), incorporated herein by reference. Formulations and uses of GLA-AF are disclosed in U.S. patent application Ser. No. 11/862,122 and WO2008/1535411, incorporated herein by reference.

CN54gp140 solution comprises CN54gp140 recombinant glycoprotein formulated in an aqueous dilution buffer. The HIV-derived amino acid sequence of CN54gp140, as predicted from the primary DNA sequence of the clone, comprises 634 residues—shown below in bold in FIG. 2. At the N-terminus there are a small number of residues—shown in FIG. 2 underlined—encoded by the human tissue plasminogen activator (tPA) signal sequence, which was included in the DNA construct to enable secretion of expressed gp140 from Chinese hamster ovary (CHO) cells during the fermentation production process. Most of the tPA signal sequence is cleaved from the expressed gp140 on exit from the CHO cell. N-terminal sequencing of the purified recombinant protein showed that the monomers are essentially a mixture of two species, one starting with SQEIHARF . . . and the other with GARSGNLW . . . meaning that either 14 or 4 amino acids of the tPA signal sequence, respectively, are present in addition to the HIV-derived sequence. These human-derived residues increase the total sequence length of CN54gp140 to 638 and 648 residues.

The molecular mass predicted by the polypeptide sequence alone is approximately 70 kD. However, the protein is heavily glycosylated and has a mass of approximately 140 kD as determined by SDS-PAGE and size-exclusion chromatography. Furthermore, the CN54gp140 secreted by CHO cells is oligomeric, and following purification is essentially trimeric, with a projected mass of 420 kD.

CN54gp140 solution is provided at a concentration of 0.50 mg/mL, as a clear, colourless, sterile liquid, presented in translucent polypropylene vials. The fill volume is 0.30 mL/vial. The vial contents of CN54gp140 solution are shown in Table 4.

TABLE 4
Vial contents of CN54gp140 solution, 0.30 mL fill

| Active ingredient | Amount | |
|---|---|---|
| CN54gp140 at 0.50 mg/mL (0.30 mL fill) | 0.150 | mg |
| Excipients | Amount | |
| Dilution buffer, pH 7.5 | | |
| Tromethamine (Tris) | 20 | mM |
| Sodium Chloride | 150 | mM |

| | |
|---|---|
| Sterile Water for Injection (WFI) | qs to volume |

The CN54gp140 solution is stored at 2-8C. The specification for the final drug product is shown below in Table 5.

TABLE 5
Specification CN54gp140 Drug Product solution

| Test Method | Specification |
|---|---|
| Appearance | Clear and colourless |
| pH value | 7.4 ± 1.0 |
| Osmolality | >240 mOsmol/kg |
| gp140 Content (OD₂₈₀ₙₘ) | 0.5 mg/ml |
| SDS-PAGE (reducing, | bands corresponding in position with |
| non-reducing) | molecular weight marker |
| Native PAGE | bands corresponding to bands obtained |
| | with the reference standard |
| Western Blot | positive reaction of gp140 protein bands |
| | with specific antibody |
| Purity (SEC-HPLC) | fragments and monomers <10% |
| | tetramers and aggregates <20% |
| | higher molecular aggregates <4% |
| Endotoxin Content (LAL) | ≦20 EU/mg gp140 |
| Sterility | Sterile |

i. GLA-AF Adjuvant Solution

GLA-AF is an aqueous formulation of GLA. The chemical properties of GLA are as follows.
MW: 1,762.311
Chemical formula: C₉₆H₁₈₄N₃O₂₂P
Production method: Chemical synthesis
Molecule type: Lipopolysaccharide
Form: Salt with ammonium counter ion

GLA is a completely synthetic 3-O-desacyl-4′-monophosphoryl lipid A (MPL®) like molecule. MPL is a lipid component of an endotoxin held responsible for toxicity of Gram-negative bacteria, which is sensed by the human immune system and is critical for the onset of immune responses to Gram-negative infection. MPL is an active ingredient of GlaxoSmithKline's proprietary adjuvant, AS04, which is a component of Cervarix—a vaccine against certain types of cancer-causing human papillomavirus (HPV), licensed for use in the European Union.

The structural formula of the bulk ammonium salt form of GLA is shown in FIG. 3.

The GLA-AF adjuvant comprises GLA, with the excipients dipalmitoylphosphatidylcholine (DPPC) and sterile water for injection. GLA-AF is a clear, colourless (to slightly hazy), sterile liquid, presented in clear glass vials, with a butyl rubber stopper and aluminium crimp seal, with a 0.5 mL fill. The vial contents of GLA-AF are shown in Table 6.

TABLE 6
Vial contents of GLA-AF, 0.5 mL fill

| Active ingredient | Amount |
|---|---|
| GLA (at 25 μg/mL) | 12.5 μg |
| Excipients | Amount |
| Dipalmitoylphosphatidylcholine (DPPC) | 10.5 μg |
| Sterile Water for Injection (WFI) | qs to volume |

The vials containing GLA-AF must be stored at 2-8° C. The specification for the GLA-AF adjuvant solution is shown below in Table 7.

TABLE 7
Specification for GLA-AF adjuvant solution

| Test Method | Specification |
|---|---|
| Appearance | Clear, to slightly hazy colourless liquid |
| | No signs of obvious contamination or foreign matter |
| | Vial should be sealed with a stopper and aluminium |
| | crimp seal |
| | Label is correctly placed and legible |
| | Label correctly identifies product by description, part |
| | and lot number |
| pH value | For Information; 7.0 ± 0.2 |
| Particle Size | 100 ± 40 nm |
| GLA quantification | 20-30 μg/ml |
| (HPLC) | |
| Residual Solvent | <60 ppm chloroform |
| Pyrogenicity | Non-pyrogenic at 1 μg GLA/kg; Pass |
| General Safety | No adverse reaction or weight loss in test species; Pass |
| Bioburden | For Information; <1 CFU/ml |
| (pre-filtered bulk) | |
| Sterility | No growth on FTM or TSB; Sterile |

TABLE 8
Vial contents of CN54gp140 IM final formulation, 0.6 mL fill,
and composition of the 0.4 mL administered dose

| | | | Amount in | |
|---|---|---|---|---|
| | Amount | | administered | |
| Active ingredient (concentration) | in vial | | dose | |
| CN54gp140 (250 μg/mL) | 150 | μg | 100 | μg |
| GLA (12.5 μg/mL) | 7.5 | μg | 5 | μg |
| | | | Amount in | |
| | Amount | | administered | |
| Excipients | in vial | | dose | |
| DPPC | 6.30 | μg | 4.2 | μg |
| Sterile water for injection | 0.30 | mL | 0.2 | mL |
| Dilution buffer (20 mM Tris, | 0.30 | mL | 0.2 | mL |

0.15M NaCl, pH 7.5)

Toxicology and Safety of rGP140 and rGP140/GLA:

The systemic toxic potential, vaginal irritancy and immunogenicity of GPGP140 vaccine candidate were assessed twice in New Zealand White rabbits. An 8 weeks toxicity and tolerance study of the product combined with GLA in rabbits by intramuscular, intranasal or intravaginal administration was also performed. These three studies concluded that administration of the product by these routes with or without adjuvant caused only local inflammation at the sites of administration and other minor physiological responses. There was no evidence of systemic toxicity. 17 healthy women received 9 intravaginal vaccinations of 100 ug rGP140 in 3 mL carbopol without serious adverse events in any of the participants. There is a large literature supporting the safety of recombinant ENV gp120 HIV sub-unit vaccines (3, 4). GLA has been used in one clinical trial to date. Volunteers received one IM immunisation with fluzone in GLA formulated as an oil emulsion (GLA-SE) in a dosing study. The highest concentration of GLA-SE (5 ug/mL) was at the threshold of tolerability, and this was attributed to the emulsion formulation (Investigator Brochure). There was no concern with four doses of IM rGP140 and 5 ug/mL GLA in a pre-clinical toxicology safety study, and clinical safety data will become available in 2011 from the MucoVac2 trial (UK HVC 001, see section 12.2) and HIVIS 08 (see section 7).

### EXAMPLE 2

### HIV CN54gp140+GLA Significantly Enhances Vaccine Antigen-Specific T and B Cell Immune Responses after Priming with DNA and MVA

Using a unique vaccine antigen matched and single Clade C approach we have assessed the immunogenicity of a DNA-poxvirus-protein strategy in mice, administering MVA and protein immunizations either sequentially or simultaneously and in the presence of a novel TLR4 adjuvant (GLA). Groups of 10 BALB/c mice were vaccinated with combinations of HIV env/gag-pol-nef plasmid DNA followed by MVA-C(HIV env/gag-pol-nef) with HIV CN54gp140 protein adjuvanted or unadjuvanted with GLA (aqueous) and either co-administered with MVA-C or given sequentially at 3 or 6 weeks. Mice were sampled (serum and vaginal wash) prior to each vaccination and three weeks post final immunization. Antigen-specific IgG and IgA production was assessed in the sera and mucosal lavage samples by quantitative ELISA. Splenocytes were harvested at necropsy and analysed for antigen-specific T cell responses using peptide pools for Env and Gag by IFN-gamma ELISpot assay. A GLA adjuvanted HIV CN54gp140 protein boost substantially enhanced the antigen-specific antibody responses in animals that received DNA-MVA or MVA priming alone. Antibody responses were similar irrespective of giving a protein envelope boost three weeks following an MVA immunization or simultaneously. Importantly, co-administration of MVA-C with HIV CN54gp140 protein adjuvanted with GLA significantly augmented the antigen-specific T cell responses to Gag peptide pools. We have demonstrated that co-administration of MVA and GLA adjuvanted HIV CN54gp140 protein was equally effective to a sequential vaccination modality. This vaccine schedule shortens the duration of and simplifies the immunization regime. In addition a significant benefit of the combined inoculation was that T cell responses to proteins expressed by the MVA-C were potently enhanced, an effect that was likely due to enhanced immunostimulation in the presence of systemic GLA.

### Methodology

The recombinant trimeric HIV-1 gp140, plasmid DNAs and MVA vector are described in Example 1.

Female BALB/c mice (Harlan, UK), 6-8 weeks old, were placed into groups (n=10) and housed in a fully acclimatized room. All animals were handled and procedures performed in accordance with the UK Home Office Animals (Scientific Procedures) Act 1986. Food and water were supplied ad libitum. Mice were immunized at 3 week intervals with three intramuscular plasmid DNA vaccinations (two plasmids, one containing an HIV CN54gp140 Env insert and the other HIV ZM96 Gag, Pot and Nef inserts), followed by 6 weeks where the animals did not receive a vaccination and then either two or four further vaccinations at 3 week intervals where the animals were injected intramuscularly with various combinations of a recombinant poxvirus (MVA env-gag-pol-nef) and/or recombinant gp140 with and without the TLR4 agonist GLA. Tail bleeds were collected before the start of the protocol and one day prior to each vaccination without anticoagulant and centrifuged in a Heraeus Biofuge pico (Fisher, UK) at 1000 g for 10 min. The serum was harvested and transferred into fresh 0.5 ml micro-centrifuge tubes (Starlabs, UK), and stored at −20° C. until antigen-specific antibody concentrations were determined by indirect quantitative ELISA. Vaginal lavage was carried out immediately before the tail bleeds using three 25 μl washes/mouse with PBS that were subsequently pooled. Lavage samples were incubated for 30 min with 4 μl of 25× stock solution protease inhibitor (Roche Diagnostics, Germany) before centrifuging at 1000 g for 10 min. The fluid supernatant from these treated samples was then transferred into a fresh 0.5 ml micro-centrifuge tube, and stored at −20° C. until antigen-specific and the total non-specific antibody concentrations were determined by indirect quantitative ELISA.

Anti-CN54gp140 Antibody Quantative ELISA

Antigen-specific gp140 binding antibodies against recombinant CN54gp140 were measured using a standardized ELISA. Maxisorp high binding 96-well plates were coated with 100 μl recombinant CN54 gp140 at 5 μg/ml in PBS for overnight at 4° C. The standard immunoglobulins were captured with a combination of anti-murine lambda and kappa light chain specific antibodies. These capture antibodies were coated onto the maxisorp plates overnight at 4° C. (100 ul of a 1:3200 dilution; Serotech). Coated plates were washed three times in PBS-T before blocking with 200 μl PBS-T containing 1% bovine serum albumin for 1 hour at 37° C. After further washing, sera diluted 1/100 or mucosal wash samples diluted 1/10 were added to the antigen coated wells and a standard titration of immunoglobulin standards added to the kappa/lambda capture antibody coated wells at 50 μl/well and incubated for 1 hour at 37° C. Plates were washed four times before the addition of 100 μl of a 1/4,000 dilution of goat anti-mouse Ig-HRP (various isotypes—matched to the standard immunoglobulin isotype; Southern Biotech) secondary antibody and incubated for 1 hour at 37° C. The plates were washed four times and developed with 50 μl/well of KPL SureBlue TMB substrate (Insight Biotechnology, UK). The IgA isotype, that was biotin labeled, required a further Streptavidin-HRP (R&D systems) amplification step prior to TMB development. The reaction was stopped after 15 min by adding 50 μl/well 1 M H₂SO₄, and the absorbance read at 450 nm on a KC4 spectrophotometer.

### IFN-Gamma T Cell ELISpot

Antigen-reactive T cells were enumerated using a standardised IFN-gamma T cell ELISpot (Shattock lab SOP). Briefly, IFN-gamma capture antibody was coated overnight on ethanol activated HIS multiscreen (Millipore) plates at 4° C., then washed with sterile PBS. 50 ul of splenocytes (5×10⁶ cells/ml) were added to each well together with various Env or Gag peptide pools or positive and negative controls of ConA (final 5 ug/ml) or medium alone and incubated for 16-18 hrs before further washing and development of the spots using a streptavidin labelled IFN-gamma sandwich detection antibody and streptavidin alkaline phosphatase. Colour was developed with the BCIP/NBT substrate solution.

### Immunization Schedule

| | | Week | | | | | | | | | |
|---|---|---|---|---|---|---|---|---|---|---|---|
| | | 0 | 3 | 6 | 9 | 12 | 15 | 18 | 21 | 24 | |
| | | Day | | | | | | | | | |
| Group Designation | | 0 | 20 | 41 | 62 | 83 | 105 | 131 | 155 | 176 | |
| A | DNA-MVA- | DNA | DNA | DNA | — | MVA | MVA | gp140 + GLA | gp140 + GLA | End test | Final report |
| | gp 140/GLA | | | | | | | | | | |
| B | DNA- | DNA | DNA | DNA | — | MVA/ | MVA/ | End test | | | Final report |
| | MVA/gp 140 + GLA | | | | | gp140 + GLA | gp140 + GLA | | | | |
| C | DNA-gp 140 + GLA | DNA | DNA | DNA | — | gp140 + GLA | gp140 + GLA | End test | | | Final report |
| D | DNA-MVA/gp 140 | DNA | DNA | DNA | — | MVA/gp140 | MVA/gp140 | End test | | | Final report |
| E | MVA-gp 140/GLA | — | — | — | | MVA | MVA | gp140 + GLA | gp140 + GLA | End test | Final report |
| F | MVA/gp 140 + GLA | — | — | — | | MVA/ | MVA/ | End test | | | Final report |
| | | | | | | gp140 + GLA | gp140 + GLA | | | | |
| G | DNA | DNA | DNA | DNA | — | | | End test | | | Final report |

Murine study was initiated to provide additional supportive data—aimed to investigate and measure the relative contribution of each component of the vaccine modality and the potential benefit of the GLA TLR₄ agonist.

Mice that did not receive plasmid DNA priming were effectively naïve until week 12.

### Results

FIG. 11 shows gp140-Specific IgG responses in animals that had been primed with DNA. GLA adjuvanted CN54gp140 substantially enhanced the antigen-specific antibody responses in animals primed with DNA and MVA. Administration of MVA and CN54gp140 protein at the same time did not significantly affect antigen-specific antibody responses. * This group went on to receive adjuvanted gp140 after day 131 (FIG. 12). FIG. 12: gp140-Specific IgG responses in animals that were primed with DNA compared to DNA unprimed animals. This chart demonstrates that the plasmid DNA has a subtle effect on the kinetics of antigen-specific antibody elicitation, however two immunizations of GLA-adjuvanted recombinant protein (days 131 and 155) produced the same level of response.

FIG. 13 shows antigen-specific serum IgA and mucosal IgG responses. The various DNA-poxvirus-protein vaccine combinations elicited serum IgA and low-level mucosal IgG responses by Week 18 post first DNA vaccination. We analysed the IFN-gamma ELISpot responses to Env and Gag antigen peptide pools (15-mer peptides overlapping by 11 aa) in splenocytes harvested from the mice at the end of the immunization schedule. FIG. 14 shows splenocyte IFN-gamma responses to HIV CN54 Env and Gag peptide pools. The T cell responses were highest in those animals that had received a DNA prime followed by a GLA adjuvanted gp140 protein boost either sequentially or in concert with MVA.

FIG. 15 shows significant augmentation of MVA elicited Gag responses in the presence of the GLA adjuvant. Importantly, significant augmention (p=0.0474 Mann-Witney) of antigen-specific T cell responses to Gag peptide pools was observed only in the group where MVA and adjuvanted CN54gp140 were co-administered.

### CONCLUSIONS

GLA adjuvanted CN54gp140 is able to significantly boost vaccine vector-derived antigen antibody responses. Co-administration of MVA and adjuvanted protein was equally effective to a sequential vaccination modality. This vaccine schedule shortens the duration of, and simplifies the immunization regime, both central to long-term vaccine feasibility. In addition, a significant benefit of the combined inoculation was that T cell responses to proteins expressed by the MVA were potently enhanced, an effect that was likely due to the presence of systemic GLA

### EXAMPLE 3

### Protocol for Raising an Immune Response in a Human to HIV

In summary, the protocol uses two prime-boost strategies using GMP products. The priming is done with DNA followed by modified pox (MVA) and boost with rGP140 protein adjuvanted with GLA. A comparison is made of 3×DNA followed by 2×MVA and 2× rGP140/GLA, versus 3× DNA followed by concurrent injection of MVA/rGP140/GLA in an accelerated schedule, with primary end-points of ENV binding antibody titre and safety and secondary end-points of neutralisation and cellular immunity. This example provides a novel regimen of Clade-C DNA (CN54 env (GP140) & ZM96 gag-pol-net), MVA (CN54 gag-pol-nef-env (GP120)) and trimeric CN54 rGP140 with GLA-AF aimed at inducing durable high titre of HIV-1 specific binding and neutralising antibody responses in an accelerated regimen.

The protocol is designed to produce an accelerated, potent regimen that is safe. All participants receive a vaccination regimen consisting of DNA-C, MVA-C, and the rGP140 protein with GLA adjuvant (see FIG. 1). The control group receives 3×DNA (weeks 0, 4, 8) followed by 2×MVA (week 16 & 20) and 2× rGP140 with GLA (weeks 24 & 28); the accelerated group receive 3×DNA (weeks 0, 4, 8) followed by 2×MVA-C and rGP140 with GLA in different arms (weeks 16 & 20). The primary endpoints for safety and immunogenicity of each regimen are 4 weeks following the last immunisation, and these are described below. There is a final visit at week 48 for all participants to determine durability of responses, and consent is collected for a week 72 specimen, so that this can be collected if warranted.

The chosen primary immunological endpoint is the titre of binding antibodies to a trimeric form of the viral ENV (CN54 GP140). Antibodies have long been known to play a role in protection and there has been renewed interest in their role in light of RV144 since CD4+ T-cell cytokine responses were seen in 33% of individuals, CD8+ cytokine responses in only 6%, and 17% had γ-IFN responses but binding antibodies were seen in 99% of vaccinees, 31% of whom were protected (1). Whilst there is no correlate of protection against HIV infection in man, passive transfer of antibodies has been shown to be protective in animal models and this is widely regarded as a hallmark of many successful anti-viral vaccines. Several studies demonstrate that vaccine induced neutralising antibody responses can confer complete protection against homologous SHIV challenge in macaques indicating that a vaccine capable of eliciting sufficient levels of Nabs could prevent establishment of infection (18). Antibody assays are carried out in the cGLP accredited Core Immunology Lab at SGUL and the cellular assays at the IAVI core Lab.

We expect that all vaccinees receiving the DNA/MVAC/rGP140 combination will develop a binding antibody response. Results reported from the RV144 trial provide the basis for the sample size calculation: In the vaccinated participants, the reciprocal geometric mean titre of binding antibodies against ENV GP120 two weeks after vaccination was about 15,000 (log 10: 4.18). An immunologically relevant improvement in this response would be a 4× increase in the geometric mean titre which on a log 10 scale translates to a difference of 0.6. Assuming a standard deviation of 0.58 on the log 10 scale (i.e. 20,000 on the natural scale), 20 participants per group are needed to detect a difference between 4.18 and 4.78 with 90% power.

For each participant, the titre of binding antibodies at weeks 24 and 32 for the accelerated and control groups respectively are derived from replicate readings. For group comparisons, the reciprocal titre are analysed on a log 10 scale and the geometric mean titre used for descriptive purposes. Nonparametric methods for group comparisons might be used if more appropriate. Other titres (secondary immunogenicity endpoints) are analysed similarly. Categorical outcomes such as the proportion of vaccinees with IFNγ T-cell responses are compared using a Chi-square or Fisher test, whichever is more appropriate. Safety endpoints are expressed as the proportion of participants that experienced an endpoint as defined below with a confidence interval.

### Primary Endpoints

i. Immunological.

The Primary immunological end-point is the geometric mean titre of binding antibodies to GP140 measured in serum 4 weeks after the final immunisation.

ii. Safety.

The primary safety parameters are graded based on systems in use at the MRC CTU, IAVI and NIH Division of AIDS and defined as:

Grade 3 or above local adverse event (pain, cutaneous reactions including induration).

Grade 3 or above systemic adverse event (temperature, chills, headache, nausea, vomiting, malaise, and myalgia).

Grade 3 or above other clinical or laboratory adverse event confirmed at examination or on repeat testing respectively.

Any event attributable to vaccine leading to discontinuation of the immunisation regimen. Data on local and systemic events listed above are solicited with specific questions or using a diary card for a minimum of 7 days following each immunisation. Data on other clinical events and laboratory events are collected with an open question at each visit and through routine scheduled investigations respectively.

### Secondary Endpoints

i. Immunological

Proportion of individuals making homologous and heterologous neutralising antibodies (Nab) and the mean titre in those that do. Neutralisation to be defined (i) as a minimum, the ability to neutralise one or more tier 1 viruses (Tier 1C MW966.5, Tier 1B SF162.LS and Tier 1A DJ263.8) and (ii) neutralisation across a panel of six transmitted founder clade C virus

The proportion of individuals who make IFNγ T cell responses (determined using Elispot assays) in response to peptide pools.

The mean number of CD8/CD4+ T-cell Spot Forming Units (SFU) per million cells in response to peptide pools

The mean percentage of CD4/CD8+ T cells producing IL-2 and/or IFNγ following ex-vivo stimulation with HIV-1 peptide pools

ADCC or ADCVI assays (if validated)

ii. Safety

All grade 1 and 2 solicited adverse events.

All other events including those considered unrelated.

We expect the results of the use of the protocol to confirm that the DNA prime, MVA boost, rGP140 reboost strategy defined herein is safe and immunogenic. We anticipate the immune responses to be broader and of higher titres than those observed using other trials, and specifically in terms of the production of ENV neutralising antibodies. We also expect the use of the protocol to show the additional adjuvanting potential of the MVA-C when co-injected with the rGP140/GLA.

### REFERENCES

1. MMWR. http://www.cdc.gov/mmwr/preview/mmwrhtml/00056803.htm (1999).
2. S. Rerks-Ngarm et al., N Engl J Med 361, 2209 (Dec. 3, 2009).
3. N. M. Flynn et al., J Infect Dis 191, 654 (Mar. 1, 2005).
4. P. Pitisuttithum et al., J Infect Dis 194, 1661 (Dec. 15, 2006).
5. M. I. Johnston, A. S. Fauci, N Engl J Med 356, 2073 (May 17, 2007).
6. S. P. Buchbinder et al, Lancet 372, 1881 (Nov. 29, 2008).
7. M. P. D'Souza, N. Frahm, AIDS 24, 803 (March 27).
8. P. A. Bart et al, Vaccine 26, 3153 (Jun. 13, 2008).
9. A. Harari et al., J Exp Med 205, 63 (Jan. 21, 2008).
10. Y. Levy et al., 17th Conference on Retrovirus & Opportunistic Infections (San Francisco
2010).
11. E. Sandstrom et al, J Infect Dis 198, 1482 (Nov. 15, 2008).
12. A. Brave. (2010).
13. M. Kim et al., AIDS Res Hum Retroviruses 21, 58 (January, 2005).
14. Y. Li et al., J Virol 80, 1414 (February, 2006).
15. A. Morner et al., J Virol 83, 540 (January, 2009).
16. D. H. Barouch et al., J Virol 79, 8828 (July, 2005).
17. R. Sealy et al., Int Rev Immunol 28, 49 (2009).
18. L. L. Baum, Curr HIV/AIDS Rep 7, 11 (February, 2010).
C. M. Rodenburg, Y. Li, S. A. Trask, Y. Chen, J. Decker, D. L. Robertson, M. L. Kalish, G. M. Shaw, S. Allen, B. H. Hahn, F. Gao, Near full-length clones and reference sequences for subtype C isolates of HIV type 1 from three different continents, AIDS Res Hum Retroviruses. 17 (2001) 161-168.
L. Su, M. Graf, Y. Zhang, H. von Briesen, H. Xing, J. Kostler, H. Melzl, H. Wolf, Y. Shao, R. Wagner, Characterization of a virtually full-length human immunodeficiency virus type 1 genome of a prevalent intersubtype (C/B′) recombinant strain in China, J Virol. 74 (2000) 11367-11376.
M. P. Cranage, C. A. Fraser, Z. Stevens, J. Huting, M. Chang, S. A. Jeffs, M. S. Seaman, A. Cope, T. Cole, R. J. Shattock, Repeated vaginal administration of trimeric HIV-1 clade C gp140 induces serum and mucosal antibody responses, Mucosal Immunol. 3 (2010) 57-68.
G. Krashias, A. K. Simon, F. Wegmann, W. L. Kok, L. P. Ho, D. Stevens, J. Skehel, J. L. Heeney, A. E. Moghaddam, Q. J. Sattentau, Potent adaptive immune responses induced against HIV-1 gp140 and influenza virus HA by a polyanionic carbomer, Vaccine. 28 (2010) 2482-2489.

### EXAMPLE 4

### Further Protocol for Raising an Immune Response in a Human to HIV

This example relates to the planned clinical trial termed UKHVC 003: A Phase I clinical trial investigating immunisation strategies using DNA, MVA and CN54rgp140 in order to maximise antibody responses: EUDRACT 2012-003277-26

### Abstract and Summary of Trial Design

### (a) Type of Design

UKHVC 003 is a randomised Phase I single centre study which will explore the impact of shortening a vaccination regimen using DNA, MVA and CN54rgp140 adjuvanted with GLA-AF in 40 healthy male and female volunteers. All volunteers will be primed three times with 24 mg DNA given over 8 weeks given in 6 injections. One group of 20 will receive an accelerated regimen and boosted twice with MVA and CN54rgp140/GLA-AF given during the same visit, but in different arms. The other group will receive the same vaccines separately and will be boosted twice with MVA and then twice more with CN54rgp140 in GLA-AF.

(b) Disease/participants studied

40 healthy male and female volunteers 18 to 45 years old who are at low risk of HIV infection are to be recruited.

(c) Trial interventions

A regimen consisting of 8 mg DNA (2 plasmids) given in 2 mls at 0, 4, 8 weeks (24 mg in total), followed by 1.10⁸ TCID₅₀ MVA given in 0.5 mls at 16, 20 weeks and 100 μg CN54rgp140 and 5 μg GLA-AF (mixed at the bedside and given in 0.4 mls) at 24, 28 weeks will be compared with one consisting of 8 mg DNA at 0, 4, 8 weeks, followed by 1.10⁸ TCID₅₀ MVA given at the same time as 100 μg CN54rgp140/5 μg GLA-AF (mixed at the bedside and given in 0.4 mls) at 16, 20 weeks during the same visit but into opposite arms.

TABLE 1
Schedule of doses, formulation and routes of immunisation

| | Route of immunisation; dose of vaccine | | |
|---|---|---|---|
| Group | week 0, 4, 8 | week 16, 20 | week 24, 28 |
| 1 | 8 mg DNA | 1 · 10⁸ TCID₅₀ MVA-C in | nothing |
| n = 20 | in 2 ml * | 0.5ml * + | |
| | IM | (100 μg CN54gp140 + | |
| | | 5 μg GLA-AF) in 0.4 ml ** | |
| | | IM | |
| 2 | 8 mg DNA | 1.10⁸ TCID₅₀ MVA-C in | (100 μg CN54gp140 + |
| n = 20 | in 2 ml * | 0.5 ml * | 5 μg GLA-AF) |
| | IM | IM | in 0.4 ml ** IM |

* 1 ml into each of the right and left upper arm muscles
** in to muscle of upper left arm

### (d) Objectives and Outcome Measures

The primary objective is to address the hypothesis that an accelerated regimen, in which MVA and CN54rgp140/GLA-AF are administered during the same rather than during sequential visits does not compromise safety or immunogenicity but rather augments the titres of systemic rgp140-specific binding and neutralising antibodies.

### (e) Outcome Measures

Immunogenicity
Frequency and magnitude (reciprocal mean endpoint titre and concentration) of systemic IgG and IgA binding antibody responses to CN54rgp140 measured 2 weeks after the final immunisation.
A Frequency and magnitude (reciprocal mean endpoint titre) of homologous and heterologous neutralising antibody (Nab) responses measured 2 weeks after the final immunisation.
A Frequency and magnitude of T-cell and B-cell responses (ELISPOT and ICS) measured 2 weeks after the final immunisation to HIV peptide pools to which there was no response at baseline.
Frequency and magnitude (reciprocal mean endpoint titre and concentration) of mucosal IgG and IgA antibody responses to CN54rgp140 measured 2 weeks after the final immunisation.

Frequency and magnitude of antibody-dependent cell-mediated cytotoxicity (ADCC) or Antibody-dependent cell-mediated viral inhibitory (ADCVI).

### Flow Diagram

Screening, Randomisation, Immunisations and Follow-Up:

### Background

### (a) the Global HIV-1 Situation and the Need for a Vaccine

The global AIDS epidemic continues to grow. In its 2011 Report on the global AIDS epidemic, UNAIDS published the following estimates for 2011: 34.2 million people were living with HIV
There were 6900 new infections per day
Women were disproportionately susceptible to infection within sub-Saharan Africa, acquiring 60% of the infections
Gains in expanding access to HIV treatment cannot be sustained without a reduction in the rate of new HIV infections

The International AIDS Vaccine Initiative (IAVI) has estimated that a vaccine has the potential to prevent over 70 million infections in 15 years.

### (b) the Status of the Field

The first clinical trials of candidate HIV vaccines started over 20 years ago and despite sustained effort and four efficacy trials, there had been virtually no good news until 2009 when RV144—the “Thai trial” reported modest protection in a cohort of low risk, predominantly heterosexual individuals [1]. RV144 was a community-based, randomised, multicentre, double-blind, placebo-controlled trial consisting of four “priming” injections of a recombinant canarypox vector (ALVAC-HIV expressing gag pro and env genes) followed by two “boosting” injections with vaccine AIDSVAX B/E Env protein given together with the last two injections of ALVAC in ALUM. In the modified intention-to-treat analysis involving 16,395 subjects, the vaccine efficacy in terms of preventing acquisition of HIV was 31.2% (95% CI, 1.1-52.1; p=0.04). Vaccination did not affect viraemia or the CD4+ T-cell count in those who subsequently became infected (see Appendix 1 for more detail). Levels of two distinct types of serum antibody have been shown to correlate with the risk of infection: IgG against the V1V/2 loop of the viral envelope was negatively correlated with risk of infection whilst levels of monomeric serum IgA with the same specificity were positively correlated with risk of infection[2]. The relatively modest effect size was seen as a major step forward in vaccine research, providing the first suggestion that the development of a safe and effective preventive HIV vaccine was possible. Interestingly, both ALVAC and AIDSVAX failed to show efficacy in previous trials and the only novel component of the trial design was the fact that the two were combined for the last two immunisations endorsing such so-called “combination strategies”. (see Appendix VI for more information)

### (c) Current Vaccination Strategies

There have always been strong proponents of vaccines focussed on either B-cell or T-cell immune responses and the history of vaccine research has reflected this. RV144 has endorsed a vaccine strategy based on the development of a more balanced immune response and efforts are now also focussed on increasing the potency and quality of adaptive immune responses which are present concurrently with, if not before HIV-1 transmission occurs [3, 4]. Many groups are exploring multicomponent heterologous prime-boost vaccine regimens which include an adjuvanted envelope glycoprotein (also refer to Appendix VI for more information).

### (d) Heterologous Prime Boost Vaccination

Heterologous prime boost regimens involve priming the immune system to (a) target antigen(s) delivered in one vaccine followed by selective boosting of the response by repeated administration of the antigen(s) delivered in a second, distinct vaccine[5]. Studies in non-human primates showed that priming with DNA followed by boosting with modified viruses such as Adenovirus (Ad5) or recombinant MVA could reduce challenge virus replication and thus also prevent the development of SIV-induced disease[6-9]. Several groups are in the process of optimising heterologous prime-boost regimens for use in humans using a variety of DNA plasmids and modified viral vectors. The strategy has proved particularly potent for T-cell responses—with more mixed success seen in stimulating antibody responses [10-16]. In line with the renewed focus in the B-cell response to envelope glycoproteins, several groups now plan to incorporate adjuvanted envelope proteins into existing prime boost regimens and, as discussed above, there is a particular interest in the role of non-neutralising antibodies with specificity for the V1V/2 loop of the viral envelope

### Supportive Murine Immunogencity Study

In a supportive mouse immunogenicity study using the same products, the advantage of the accelerated schedule combining MVA and CN54rgp140/GLA-AF was very clear (see for example earlier examples). Groups of 10 mice were immunised 3 times with DNA at 0, 3 and 6 weeks and then boosted either with 10⁷ MVA at weeks 12 and 15 and then with 20 μg CN54rgp140/204 GLA-AF at weeks 18 and 21 (group 1) or with MVA and 20 μg rgp140/20 μg GLA-AF given in opposite legs at the same time, at weeks 12 and 15 (group 2). Antibody responses to CN54rgp140 were low in both of these groups before boosting with the protein but developed rapidly after that (week 12). Co-administration of the adjuvanted protein with MVA did not result in significantly enhanced antibody responses, which were very high in both groups and had probably plateaued—potentially obscuring any differences. Such combination of the vaccines did however significantly augment the T-cell responses to GAG peptides—suggesting a systemic role for GLA-AF (adjuvanting the responses to the antigens expressed only by the DNA and MVA vaccines).

In the absence of DNA priming, rather than augmenting responses, co-injection of MVA and CN54rgp140/GLA-AF resulted in lower responses than were seen to the same vaccines given sequentially—suggesting that the total number of exposures of the immune system to products of the viral envelope might may be critical to the regulation of both the magnitude and kinetics of the humoral responses which develop. The CN54 envelope is a component of all three of the vaccines and it seems likely from these data that both the number of vaccinations and the spacing between them plays a role in the kinetics of the responses which develop. This interpretation is further endorsed by the observation that in the absence of DNA priming, CN54rgp140-specific responses were negligible after the first boost with CN54rgp140/GLA-AF and did not develop until three weeks after the second. By contrast, although ENV-specific antibody responses were undetectable after three priming injections with DNA, they developed rapidly and reached a plateau soon after the first injection with the trimeric protein. In effect, DNA priming accelerated the appearance of Ab responses to the trimeric protein. DNA priming took 8 weeks, and so this “acceleration” in the maturation of the gp140-specific Ab response offered no advantage in terms of the overall length of the vaccination schedule and also required more vaccinations. It remains possible, of course, that there were significant differences in the duration of the antibody responses between the groups but such analysis was beyond the scope of this study design.

### (i) Rabbit Toxicology Study

In a GLP rabbit tolerance and toxicity study (CR 520073) done to support the doses and regimens proposed here, there were no unacceptable local side effects or worrying indications of systemic toxicity and recovery after vaccinations were complete was as expected. All reactions seen were in accordance with what would be expected. importantly, there was no evidence that co-administration of the CN54rgp140/GLA-AF and MVA-C was associated with any increased reactogenicity—at least in terms of any of the parameters which were measured suggesting that both regimens were safe. Analysis of the (presumed) peak antibody responses measured 2 weeks after the last immunisation in each group of 13-14 rabbits strongly corroborated what had been seen in the mouse and confirmed that the accelerated regimen did not result in attenuation of the antibody response but that rather the very similar (and possibly maximal) response of ˜1500 μg/ml of systemic CN54rgp140-specific IgG could be achieved in a shorter time, demonstrating the advantage of the accelerated regimen at least in respect of this immune response.

In contrast to the mouse, the rabbit does not express a homologue of the ligand for GLA-AF (TLR4) and this should be borne in mind when comparing and interpreting the immunogenicity data from the two species and also in the extrapolation of any findings to the human situation.

### Rationale and Objective

We aim to induce durable systemic binding antibodies to CN54rgp140 at levels exceeding those seen in the RV144 trial. In addition, we propose that an accelerated regimen, with 5 rather than 7 immunisations and shorter by 8 weeks, will augment humoral responses without compromising safety. We will compare 2 vaccination regimens. Both regimens include priming three times with 8.0 mg of DNA (plasmids encoding the ZM96 Clade C gag-pol-nef and CN54 Clade C env). One group will then be boosted twice with MVA-C and then with CN54 rGP140 protein adjuvanted with GLA-AF. The other “accelerated” group will receive the MVA-C and CN54rgp140/GLA together, albeit in different arms. A similar vaccination strategy using almost identical DNA plasmids but using NYVAC expressing similarly matched Clade C inserts, proved to be particularly potent at inducing envelope-specific CD4+ T-cell responses and 93% of individuals made such responses after 3 priming injections with DNA-C at 0, 4 and 8 weeks followed by one boost with NYVAC-C. The antibody responses in the trial (EV03/ANRSvac20) were low, and only seen in a minority of individuals. By contrast, MVA CMDR has been shown to be a very potent B-cell immunogen in the HIVIS03 trial and gp160-specific antibody responses were seen in 100% of those primed three times with multiclade DNA and boosted twice with MVA, albeit at low titres (Gunnel Biberfeld personal communication). Based on our previous experiences with similar matched Clade C vaccines, we predict that we will prime a strong ENV-specific T-cell response and that boosting with CN54rgp140 will result in envelope-specific antibody responses in 100% of individuals at higher titres than seen in RV144.

Investigational product/intervention(s)

### (a) DNA

Volunteers will receive 4 mg of each of two plasmids intramuscularly (IM) in a volume of 1 ml at 0, 4 and 8 weeks. One plasmid encodes ZM96 Clade C gag-pol-nef derived from the 96ZM651-8 clone construct developed by B. H. Hahn, G. M. Shaw and F. Gao at the University of Alabama at Birmingham. The other encodes CN54 Clade C env derived from the HIV-1 97CN54 coding sequences (Geneart). Both sequence optimised insert were introduced VRC8400 CMV/R vector (NIAID/NIH). Vaccines have been manufactured by Althea Technology, Inc (USA).

### (b) MVA-C

The MVA-C has been developed by M. Esteban at the Centro Nacional de Biotecnologia of CSIC and expresses the HIV-1 protein gp120 and the fusion protein gag-pol-nef from HIV-1 97CN54. Volunteers will receive 1.10⁸ TCID₅₀ in a volume of 0.5 mls. MVA will be injected into the same arm that received the DNA and CN54rgp140/GLA-AF into the other. Vaccine has been manufactured by Bavarian Nordic (Denmark).

(c) CN54rgp140

The CN54rgp140 is a recombinant GP140 derived from the HIV-1 CN54 coding sequence and has been manufactured by Polymun, Austria. CN54rGP140, is a trimeric recombinant C-clade ENV protein, derived from a Chinese viral isolate. The protein comprises a sequence of 670 amino acids, and has been shown to be immunogenic in non-human primates and other animal models. To date MucoVac 1 (EudraCT number 2007-000781-20) is the only human clinical trial to have used the trimeric CN54rgp140 although the protein was not administered systemically but topically. Mucovac 2 will generate the first safety data on the systemic administration of the CN54rgp140 in GLA-AF in healthy volunteers. In this trial the protein will be supplied at 0.5 mg/ml in a volume of 0.3 mls. 100 μg of protein will be administered IM after bedside mixing with GLA-AF (see below).

### (d) GLA-AF

In the current trial we will use GLA-AF, an aqueous glucopyranosyl lipid A adjuvant (GLA) and a completely synthetic monophosphoryl lipid A (MPL®) like molecule. MPL is a component of human vaccines including Cervarix which is a vaccine to prevent certain types of human papilloma virus infection associated with cervical cancer and is licensed for use in the European Union. Pre-clinical studies suggest that the potency of GLA-AF is 10 fold greater than MPL in vitro [20] Both GLA and MPL adjuvant are potent stimulators of antigen presenting cells through their binding and activation of toll-like receptor 4 (TLR4). GLA has been used in one previous clinical trial, [21] in which volunteers received one IM immunisation with fluzone plus GLA formulated in emulsion (GLA-SE). Four concentrations of GLA in emulsion were studied (0.5, 1, 2.5 and 5 μg). 5 μg was administered to 4 individuals, 2 adults and 2 elderly. Three of four experienced multiple grade 2 and above adverse events, which was attributed to the emulsion formulation rather than the adjuvant per se. The lower concentrations were safe and well tolerated, with only transient, mild-to-moderate symptoms and signs, and GLA significantly enhanced the immune response to the immunogen (see IB).

As GLA-AF is the aqueous form of the adjuvant we expect it to be better tolerated than the oil emulsion. The potential toxicology of GLA-AF has been assessed in rabbits and was well tolerated [22]. Priming the immune system prior to administration of GLA-AF is not expected to alter the adverse event profile of CN54rgp140/GLA-AF because it is not thought to stimulate the adaptive immune responses that could lead to the sort of cascade of cytokines which might result in enhanced reactogenicity. Recombinant protein in GLA-AF has been given to macaques after 4×DNA immunizations and there were no overt adverse events, although this was not designed as a GLP toxicity experiment. In a GLP rabbit toxicity and tolerance study in 50 New Zealand white rabbits, done to support the dose levels and immunisation regimens above, the vaccine formulations caused no systemic toxicity or unacceptable local side effects, and induced specific antibody responses [23].

### Rationale for Interventions

### DNA Dose

The dose of DNA has been chosen in accordance with what was used in the EuroVacc trials. 4 mg of DNA was well tolerated and higher doses of other HIV vaccines have been given in other trials with no safety concerns. One major concern about the use of DNA in man is the presumed inefficiency of uptake by target cells. Methods such as electroporation aim to increase the efficiency of uptake and have been shown to enhance immunogenicity [18]. Taking all these factors into consideration, we feel that doubling the dose of DNA does not carry any increased safety concerns.

### MVA Doses

1.10⁸ TCID₅₀ will be used in accordance with the dose of NYVAC used in the EuroVacc trials and the doses of MVA used in HIVIS/TaMoVac trials (10⁸ TCID₅₀) in which there have been no concerns about safety. This dose has been shown to be well tolerated and immunogenic and has now been administered to many individuals in these and many other trials of other vaccines.

CN54 rgp140 GLA-AF Doses

We will give 100 μg CN54rgp140 in 5 μg GLA-AF. A previous randomized trial of a recombinant (monomeric) HIV rgp120 envelope protein compared the effects of 3 different adjuvants [24]. 200 μg protein was administered with 50 μg MPL50 μg QS21 (a derivative of saponin formulated with and without emulsion) and compared to rgp120 given in alum. After 3 intramuscular doses, high titre, durable antibodies were observed at comparable levels seen in HIV infected patients in both groups receiving the novel adjuvants. Adverse events were more frequent and more severe in those receiving emulsion[24]. Taking the safety data in comparable trials, and the immunogenicity in animals, it has been decided to proceed with 100 μg CN54rgp140 formulated in 5 μg GLA-AF (aqueous solution) in future trials. Safety data on the systemic use of the same formulation and doses will be available during Q2 2012 from at least the MucoVac2 trial.

### Vaccine Products

Forty participants will receive DNA, MVA and CN54rgp140 in GLA-AF vaccines in a schedule of dose, formulations and routes as described. Participants in group 1 will receive five immunisations at wk 0, 4, 8 16 and 20 and participants in group 2 will receive 7 at weeks 0, 4, 8, 16, 20, 24 and 28.

A vaccine accountability log will be kept for the study supplies throughout the study. This should be used to record the identification of the subject to whom the study vaccine was dispensed. This will be verified by the study monitor.

The date and time of administration will be recorded in the eCRF.

### IMP Manufacture and Supply

Vaccines will be supplied by Imperial College London and manufactured as below:
DNA Althea 11040 Roselle Street, San Diego, Calif. 92121, USA.
MVA Bavarian Nordic, CVR-no. 1627 11 87, Hejreskowej 10A, DK—3490, Kvistgård, Denmark
CN54rgp140 Polymun Scientific Immunbiologische Forschung GmbH, Donaustrasse 99, 3400 Klosterneuburg, Austria.
GLA-AF Infectious Disease Research Institute, 1124 Columbia Street, Suite 400, Seattle, Wash. 98104 USA

Statistical Considerations

Method of Randomisation

Participants will be block-randomised centrally using a computer-generated algorithm with a back-up manual procedure and randomisation will be stratified on the basis of gender.

### Outcome Measures

(a) Primary Outcomes

### (i) Immunogenicity

The primary immunogenicity endpoint will be the endpoint titre of systemic CN54 rgp140-specific binding IgG antibodies measured 2 weeks after the scheduled final vaccination.

### (ii) Safety

Primary safety outcomes are grade 3 or above local or systemic solicited adverse events (see section 7, Table 2) and any adverse event that results in a clinical decision to discontinue further immunisations.

(b) Secondary Outcomes

### (i) Immunogenicity

The absolute concentration of systemic and mucosal CN54 rgp140-specific IgG and IgA antibodies measured 2 weeks after the scheduled final vaccination.
The frequency and magnitude of HIV peptide-specific T-cell and B-cell ELISPOT responses using frozen PBMCs collected 2 weeks after the final vaccination. The magnitude of the response will be determined by calculating the mean number of SFC/10⁶ (from triplicate samples) and this analysis will be restricted to those samples which have been defined as positive.
The endpoint titre of systemic CN54rgp140-specific IgA and mucosal IgG and IgA antibodies measured 2 weeks after the final vaccination.

The frequency and magnitude of neutralising antibody responses to tier 1 and tier 2 viruses measured using standardised pseudovirus and PBMC assays measured 2 weeks after the final vaccination.

### (ii) Safety

Any grade of adverse event that occurs in a participant that has received at least one immunisation

(c) “Exploratory” Immunogenicity Analyses
T-cell and B-cell immune responses to the vaccine will be measured in participating laboratories and analyses will include epitope mapping, polychromatic flow cytometry and the B-cell antibody repertoire.
Systemic antibody responses to MVA will be outsourced to an accredited laboratory.

### Sample Size

### Safety

By the end of this study 20 participants will have been exposed to each schedule in groups 1 and 2, and this provides confidence around the event proportions of 0-60% as follows:

| Number of subjects | Proportion | 95% confidence |
|---|---|---|
| with event | if n = 20 | interval¹ |
| 0 | 0% | 0-17% |
| 2 | 10% | 12-32% |
| 4 | 20% | 6-44% |
| 6 | 30% | 12-54% |
| 7 | 40% | 19-64% |
| 10 | 50% | 27-73% |
| 12 | 60% | 36-81% |

¹Wilson interval (suitable for small sample sizes)

### Immunological Endpoint

The primary immunological endpoint is the endpoint titre CN54gp140-specific binding antibodies. In the RV144 trial, canarypox (ALVAC) and adjuvanted AIDSVAX (gp120) were co-administered, 99% of those vaccinated made binding antibodies to gp120 and certain responses correlated with the partial efficacy observed. We predict that all vaccinees receiving DNA, MVA and adjuvanted CN54rgp140 will develop systemic binding antibodies. In the absence of other data using systemic responses to CN54rgp140, we have based our sample size calculation on the titres of antibodies reported in RV144. In those receiving vaccine, the reciprocal geometric mean titre of binding antibodies for subtype E gp120 two weeks after the final vaccination was ˜15000 (log 10: 4.18) and for subtype B gp120 was ˜30,000 (log 10: 4.5). We have based our calculations on the lower of the two responses (subtype E). We anticipate that 100% of individuals in group 1 will make detectable systemic antibody responses to CN54rgp140 and also predict that the titre will be at least as good as reported in RV144. We propose that an immunologically relevant improvement in this response would be a four fold increase in the geometric mean titre, and this translates to a difference of 0.60 on the log 10 scale. In the absence of raw data from RV144, we have assumed a standard deviation of 0.58 on the log 10 scale in the distribution of the antibody responses (this corresponds to a sd of ˜20,000 in titres). Assuming this variation, 20 participants per group will enable the detection of a difference in titres of 4.18 and 4.78 with 90% power and 5% alpha.

### Interim Monitoring and Analyses

Analyses will be performed at the MRC CTU and ICTU.

The accumulating safety and immunogenicity data will be reviewed once by the IDMC after half of the participants have completed the immunisation schedule.

An unscheduled meeting of the IDMC may be required at the request of the TMG, in which case the IDMC will make a recommendation about whether or not to continue further immunisations.

### Data Analyses and Presentations

A full statistical analysis plan will be developed before the trial is analysed. It will be based on the following summary:

(a) Participant Populations
Intention-to-treat (ITT) population: all participants randomised and given at least one immunisation in the trial.
Per-protocol (PP) population: all participants randomised and immunised with all scheduled immunisations, and who complete the trial with no major protocol deviations.

(b) Immunogenicity Variables

### (i) Primary Immunogenicity Outcome:

The endpoint titres of systemic CN54rgp140-specific IgG antibodies will be determined by assaying serial dilutions of sera with standardised ELISA assays. Endpoint titres of antigen specific IgG antibodies will be described by time-point and group, and compared using parametric or rank tests as appropriate. Assays will be validated using a set of positive and negative control sera which will also determine assay cut-offs.

### (ii) Secondary Immunogenicity Outcomes—

The absolute levels of antibody in serum samples will be determined using a quantitative assay developed and standardised in the laboratory of Robin Shattock (Imperial College). In this sandwich capture ELISA, the Ab of interest (in this case CN54-specific IgG or IgA) is captured by the relevant target Ag and then detected using a labelled isotype specific secondary Ab. An estimate of the concentration of Ab in the sample is calculated by interpolation relative to a standard curve based on titration of purified human standard IgG or IgA captured by anti-human kappa/lambda-specific antibodies. The assay will be validated

The frequency and magnitude of IFN-γ Elispot responses will be determined using PBMCs which will be purified from whole blood, frozen and stored. Samples will be analysed in batches using the T-cell Elispot assay. Cells will be stimulated using pools of peptides derived from the vaccine and also negative and positive control stimuli. The number of spots forming cells (SFC), indicative of cytokine release (γ-IFN and/or IL-2), will be enumerated using an automated Elispot reader. A positive result will be defined relative to a pre-defined cut-off threshold value and assays will be validated using predefined thresholds based on the responses to positive and negative control stimuli. More information on the assay and definition of positive results will be supplied in the statistical analysis plan. The number of ‘responders’ in each assay will be presented by time-point and group, and compared in terms of the proportion responding using a Chi-square or exact test as appropriate.

The frequency and magnitude of antigen specific IgA and IgG antibody secreting cells (ASC) will be assessed by B-cell Elispot assay. Whole PBMC's purified from whole blood frozen and stored will be analysed in batches. Resurrected frozen cells will be stimulated with whole antigen as well as positive and negative control stimuli. The resulting number of spots indicative of antibody secretion (either IgG or IgA) will be enumerated using an automated Elispot reader. A positive result will be defined relative to pre-defined cut off levels for the assay based on thresholds determined from negative and positive stimuli. The number of responders in each assay will be presented by time point and compared by Chi-square or Fishers' exact test (as appropriate) analysis according to vaccination group.

For both assays, a ‘responder’ will be defined as a participant in whom at least one post-treatment immunogenicity variable was classified as ‘response detected’.

(iii) Exploratory Immunogenicity End Points:

Exploratory endpoints will be assayed in relevant accredited laboratories if preliminary results warrant such analyses.

### (c) Safety Variables

The original verbatim terms used by the investigator to identify AEs in the CRFs will be coded using an appropriate medical coding scheme (MedDRA). In all summaries, if a participant reports the same system organ class or preferred term more than once then the worst severity and worst relationship to trial vaccine will be taken. Discrepancies between diary card and CRF reports will be queried by the monitor. It is assumed that the grade assigned by the clinician is more accurate, and this will be the grade reported in the tables. If the diary card grade is worse, this will be foot noted.

All safety end-points will be graded by the Clinical Investigators and reviewed by the Trial Management Group.

Safety outcomes will be reported overall with proportion and 95% confidence interval, and by group and time-point, and by relationship to study product.

For the primary analysis of safety endpoints, results will be expressed as a proportion with confidence interval, and groups compared using Fisher's exact test.

### REFERENCES

1. Rerks-Ngarm, S., et al., Vaccination with ALVAC and AIDSVAX to prevent HIV-1 infection in Thailand. N Engl J Med, 2009. 361(23): p. 2209-20.
2. Haynes, B. F., Case-control study of the RV144 for immune correlates: the analysis and way forward. AIDS vaccine 2011, 1215 September 2011, Bangkok, Thailand [Abstract PL01.04], 2011.
3. McMichael, A. J., et al., The immune response during acute HIV-1 infection: clues for vaccine development. Nat Rev Immunol, 2010. 10(1): p. 11-23.
4. McElrath, M. J. and B. F. Haynes, Induction of immunity to human immunodeficiency virus type-1 by vaccination. Immunity, 2011. 33(4): p. 542-54.
5. Woodland, D. L., Jump-starting the immune system: prime-boosting comes of age. Trends Immunol, 2004. 25(2): p. 98-104.
6. Robinson, H. L. and R. R. Amara, T cell vaccines for microbial infections. Nat Med, 2005. 11(4 Suppl): p. S25-32.
7. Letvin, N. L., et al., Preserved CD4+ central memory T cells and survival in vaccinated SIV-challenged monkeys. Science, 2006. 312(5779): p. 1530-3.
8. Makitalo, B., et al., Enhanced cellular immunity and systemic control of SHIV infection by combined parenteral and mucosal administration of a DNA prime MVA boost vaccine regimen. J Gen Virol, 2004. 85(Pt 8): p. 2407-19.
9. Kim, J. H., et al., HIV vaccines: lessons learned and the way forward. Curr Opin HIV AIDS, 2010. 5(5): p. 428-34.
10. McCormack, S., et al., EV02: a Phase I trial to compare the safety and immunogenicity of HIV DNA-C prime-NYVAC-C boost to NYVAC-C alone. Vaccine, 2008. 26(25): p. 3162-74.
11. Sandstrom, E., et al., Broad immunogenicity of a multigene, multiclade HIV-1 DNA vaccine boosted with heterologous HIV-1 recombinant modified vaccinia virus Ankara. J Infect Dis, 2008. 198(10): p. 1482-90.
12. Excler, J. L., et al., A strategy for accelerating the development of preventive AIDS vaccines. Aids, 2007. 21(17): p. 2259-63.
13. Bakari, M., et al., Broad and potent immune responses to a low dose intradermal HIV-1 DNA boosted with HIV-1 recombinant MVA among healthy adults in Tanzania. Vaccine, 2011. 29(46): p. 8417-28.
14. Paris, R. M., et al., Prime-boost immunization with poxvirus or adenovirus vectors as a strategy to develop a protective vaccine for HIV-1. Expert Rev Vaccines, 2010. 9(9): p. 1055-69.
15. Harari, A., et al., An HIV-1 dade C DNA prime, NYVAC boost vaccine regimen induces reliable, polyfunctional, and long-lasting T cell responses. J Exp Med, 2008. 205(1): p. 63-77.
16. Levy, Y., et al., Optimal priming of poxvirus vector-based regimens requires 3 DNA injections; results of the randomised multicentre EV03/ANRS Vac20 Phase I/II trial, in 17th Conference on Retroviruses and Opportunisitic infections (78LB). 2010.
17. Bart, P. A., et al., EV01: a phase I trial in healthy HIV negative volunteers to evaluate a clade C HIV vaccine, NYVAC-C undertaken by the EuroVacc Consortium. Vaccine, 2008. 26(25): p. 3153-61.
18. Vasan, S., et al., In vivo electroporation enhances the immunogenicity of an HIV-1 DNA vaccine candidate in healthy volunteers. PLoS One, 2011. 6(5): p. e19252.
19. Lewis, D. J., et al., Phase I Randomised Clinical Trial of an HIV-1(CN54), Clade C, Trimeric Envelope Vaccine. Candidate Delivered Vaginally. PLoS One, 2011. 6(9): p. e25165.
20. Rallabhandi, P., et al., Differential activation of human TLR4 by Escherichia coli and Shigella flexneri 2a lipopolysaccharide: combined effects of lipid A acylation state and TLR4 polymorphisms on signaling. J Immunol, 2008. 180(2): p. 1139-47.
21. Human GLA—Muvovac2 Investigators Brochure. 2011.
22. Test facility study number 516926. Report No. 31061: 4 week intramuscualr toxicity and tolerance study of glucopyranosyl lipid adjuvant (GLA) in rats.
23. Test facility study number 516596. Report number 30962: 8 week toxicity and tolerance study of CN54gp140 in rabbits by intramuscular, intranasal or intravaginal administration.
24. McCormack, S., et al., A phase I trial in HIV negative healthy volunteers evaluating the effect of potent adjuvants on immunogenicity of a recombinant gp120W61D derived from dual tropic R5×4 HIV-1ACH320. Vaccine, 2000. 18(13): p. 1166-77.
25. Pitisuttithum, P., et al., Randomized, double-blind, placebo-controlled efficacy trial of a bivalent recombinant glycoprotein 120 HIV-1 vaccine among injection drug users in Bangkok, Thailand. J Infect Dis, 2006. 194(12): p. 1661-71.
26. Flynn, N. M., et al., Placebo-controlled phase 3 trial of a recombinant glycoprotein 120 vaccine to prevent HIV-1 infection. J Infect Dis, 2005. 191(5): p. 654-65.
27. Rowland-Jones, S., et al., HIV-specific cytotoxic T-cells in HIV-exposed but uninfected Gambian women. Nat Med, 1995. 1(1): p. 59-64.
28. Mazzoli, S., et al., HIV-specific mucosal and cellular immunity in HIV-seronegative partners of HIV-seropositive individuals. Nat Med, 1997. 3(11): p. 1250-7.
29. Wilson, N. A., et al., Vaccine-induced cellular responses control simian immunodeficiency virus replication after heterologous challenge. J Virol, 2009. 83(13): p. 6508-21.
30. Liu, J., et al., Immune control of an SIV challenge by a T-cell-based vaccine in rhesus monkeys. Nature, 2009. 457(7225): p. 87-91.
31. Chung, C., et al., Not all cytokine-producing CD8+ T cells suppress simian immunodeficiency virus replication. J Virol, 2007. 81(3): p. 1517-23.
32. Spentzou, A., et al., Viral inhibition assay: a CD8 T cell neutralization assay for use in clinical trials of HIV-1 vaccine candidates. J Infect Dis, 2010. 201(5): p. 720-9.
33. Hessell, A. J., et al., Broadly neutralizing human anti-HIV antibody 2G12 is effective in protection against mucosal SHIV challenge even at low serum neutralizing titers. PLoS Pathog, 2009. 5(5): p. e1000433.
34. Hessell, A. J., et al., Effective, low-titer antibody protection against low-dose repeated mucosal SHIV challenge in macaques. Nat Med, 2009. 15(8): p. 951-4.
35. Scheid, J. F., et al., Broad diversity of neutralizing antibodies isolated from memory B cells in HIV-infected individuals. Nature, 2009. 458(7238): p. 636-40.
36. Pancera, M., et al., Structure of HIV-1 gp120 with gp41-interactive region reveals layered envelope architecture and basis of conformational mobility. Proc Natl Acad Sci USA, 2010. 107(3): p. 1166-71.
37. Chen, L., et al., Structural basis of immune evasion at the site of CD4 attachment on HIV-1 gp120. Science, 2009. 326(5956): p. 1123-7.
38. Hubner, W., et al., Quantitative 3D video microscopy of HIV transfer across T cell virological synapses. Science, 2009. 323(5922): p. 1743-7.
39. Walker, L. M., et al., Broad and potent neutralizing antibodies from an African donor reveal a new HIV-1 vaccine target. Science, 2009. 326(5950): p. 285-9.
40. Buchbinder, S. P., et al., Efficacy assessment of a cell-mediated immunity HIV-1 vaccine (the Step Study): a double-blind, randomised, placebo-controlled, test-of-concept trial. Lancet, 2008. 372(9653): p. 1881-93.
41. D'Souza, M. P. and N. Frahm, Adenovirus 5 serotype vector-specific immunity and HIV-1 infection: a tale of T cells and antibodies. Aids, 2010. 24(6): p. 803-9.
42. Johnston, M. I. and A. S. Fauci, An HIV vaccine—challenges and prospects. N Engl J Med, 2008. 359(9): p. 888-90.
43. Sodora, D. L., et al., Toward an AIDS vaccine: lessons from natural simian immunodeficiency virus infections of African nonhuman primate hosts. Nat Med, 2009. 15(8): p. 861-5.
44. Koup, R. A., et al., Priming immunization with DNA augments immunogenicity of recombinant adenoviral vectors for both HIV-1 specific antibody and T-cell responses. PLoS One, 2010. 5(2): p. e9015.

### EXAMPLE 5

### Rabbit Toxicity and Immunogenicity Study

29 Week Intramuscular Toxicity Study of DNA/MVAC-C/Rgp140 Vaccine in the Rabbit with 4 Week Recovery Period

### Objective(S)

A prophylactic vaccine for the prevention of infection by human immunodeficiency virus (HIV) is being developed by the Sponsor. The vaccine regimen consists of a DNA-C prime, a boost with MVAC-C (a non-replicating gene therapy vector) and final treatment with the HIV coat protein rgp140 and the adjuvant glycopyranosyl lipid adjuvant (GLA).

The objectives of this study are to determine the potential toxicity of vaccine regimen when given intramuscularly over a 24 week period to rabbits, to evaluate the potential reversibility of any findings, and to provide data to support the use of this treatment regimen in humans. In addition, the biodistribution of DNA into various tissues will be assessed by quantitative polymerase chain reaction (qPCR).

Guidelines for Study Design

The design of this study was based on the study objective(s), the overall product development strategy for the test item, and the following study design guidelines:
Committee for Medicinal Products for Human Use (CHMP). Note for Guidance on Repeated Dose Toxicity. CPMP/SWP/1042/99rev1.
Committee for Proprietary Medicinal Products (CPMP). Note for Guidance on Preclinical Pharmacological and Toxicological Testing of Vaccines. CPMP/SWP/465/95.
Committee for Proprietary Medicinal Products (CPMP). Note for Guidance on the Quality, Preclinical and Clinical Aspects of Gene Transfer Medicinal Products. CPMP/BWP/3088/99.

### Test and Control Items

### Test Item(s)

Identification: CN54ENV-0937202
Identification: ZN96GPN-0927353
Identification: MVAC-C-UKHVC
Identification: CN54gp140

### Control Item(s)

Identification: Dilution buffer pH 7.5 tromethamine (Tris) 20 mM, sodium chloride 150 mM

### Control Item(s)

Identification: Phosphate buffer saline (pH 7.4)

### Adjuvant

### Identification: GLA

### Dose Formulation and Analysis

### Preparation of Control Item

The control items will be dispensed for administration to Group 1 control animals. An adequate amount of the control item will be dispensed into aliquots, which will be stored in a refrigerator set to maintain 4° C. until use.

Any residual volumes will be discarded before issue of the Final Report.

### Preparation of Test Item

A trial preparation representative of the dosing concentrations and volumes may be carried out before the start of the study to assess the suitability of the formulation procedure. Trial preparation formulations will not be used for dosing and will be discarded after the assessment is complete.

Test item dosing formulations will be prepared based on Sponsor instructions. The dosing formulations will be prepared on the day of dosing.

### (a) Preparation of DNA

There is no preparation. The DNA will be dispensed as supplied.

### (b) Preparation of MVA

There is no preparation. The MVA will be dispensed as supplied.

### (c) Preparation of GP140 and GLA

Shortly before each muscular dose is administered, the formulation will be made by mixing equal volumes (0.30 mL) of CN54gp140 and GLA in the vial that originally contained the CN54gp140 only. The final formulation is a clear, colourless liquid, and the final formulated vial should contain 0.6 mL. Sufficient volume of this formulation is then drawn into a syringe for administration of 0.4 mL. The vial contents of the formulation, and composition of the 0.4 mL administered dose, are shown below.

Vial Contents of CN54gp140 IM Final Formulation, 0.6 mL Fill, and Composition of the 0.4 mL Administered Dose

| | Amount | | Amount in | |
|---|---|---|---|---|
| Active ingredient | in | | administered | |
| (concentration) | vial | | dose | |
| CN54gp140 (500 μg/mL) | 150 | μg | 33.3 | μg |
| GLA (12.5 μg/mL) | 3.75 | μg | 2.5 | μg |
| | Amount | | Amount in | |
| | in | | administered | |
| Excipients | vial | | dose | |
| DPPC | 6.30 | μg | 4.2 | μg |
| Sterile water for injection | 0.30 | mL | 0.2 | mL |
| Dilution buffer (20 mM | 0.30 | mL | 0.2 | mL |

Tris, 0.15M NaCl, pH 7.5)

Any residual volumes will be discarded before issue of the Final Report.

### Sample Collection and Analysis

There will be no samples collected for analysis on this study. No formulation analysis will be conducted. This exception to GLP will be documented in the study report.
Test System
Species: Rabbit
Strain: New Zealand White
Source: Harlan Limited, UK
Number of Males Ordered: 21
Number of Females Ordered: 21
Target Age at the Initiation of Dosing: 11-12 weeks (approximately)
Target Weight at the Initiation of Dosing: 2.5-3 kg (approximately)

The actual age, weight, and number of animals received will be listed in the Final Report.

### Justification of Test System and Number of Animals

At this time, studies in laboratory animals provide the best available basis for extrapolation to humans and are required to support regulatory submissions. Acceptable models which do not use live animals currently do not exist.

The rabbit has been selected by the Study Director in consultation with the Sponsor as the test model:
to satisfy regulatory requirements for toxicity testing.
because of the availability of background data and proven suitability in toxicology studies.

The number of animals chosen for this study is the smallest number considered necessary to provide sufficient data.

The rabbit has previously been used in studies involving this type of vaccine.

### Selection, Assignment, and Replacement of Animals

Animals will be removed in random order from their transport boxes and allocated to dose group on arrival by placing them in separate cages. Cages will be housed on racks according to treatment and labelled with the study number, animal number and group number.

Control animals will be housed on a separate rack.

Animals suspected of being diseased will be culled from the study. If significant numbers of animals are unsuitable, the entire batch will be rejected by the Study Director and a new batch obtained.

During the week before the commencement of dosing, the animals will be approved for entry into the experiment on the basis of satisfactory clinical observation records and body weight profile.

### EXPERIMENTAL DESIGN

| | Animal Numbers | | | |
|---|---|---|---|---|
| | Main Study | | Recovery | |
| Group | M | F | M | F |
| 1 | 1-4 | 22-25 | 5-7 | 26-28 |
| 2 | 8-11 | 29-32 | 12-14 | 33-35 |
| 3 | 15-18 | 36-39 | 19-21 | 40-42 |

The treatment regimen is:

| | Week | | | |
|---|---|---|---|---|
| Group | 1 | 4 | 7 | 13 |
| 1 | Vehicle | Vehicle | Vehicle | Vehicle |
| 2 | DNA-C | DNA-C | DNA-C | MVAC-C |
| 3 | DNA-C | DNA-C | DNA-C | MVAC-C/rgp |
| | | | | 140/GLA |

| | Week | | | |
|---|---|---|---|---|
| Group | 16 | 19 | 22 | 25 |
| 1 | Vehicle | Vehicle | Vehicle | Vehicle |
| 2 | MVAC-C | rgp | rgp | rgp |
| | | 140/GLA | 140/GLA | 140/GLA |
| 3 | MVAC-C/rgp 140/GLA | | | |

### Administration of Test and Control Items

The test and control items will be administered to the appropriate rabbits by intramuscular administration as detailed in Section 13. The first day of dosing for each animal will be designated as Day 1.

Administration and sites are:

Left hind limb=Injection site 1
Right hind limb=Injection site 2

| | Week | | | | |
|---|---|---|---|---|---|
| Group | 1 | 4 | 7 | Volume | Site |
| 1 | PBS buffer | PBS buffer | PBS buffer | 1 mL | Left leg |
| 2 | CN54ENV | CN54ENV | CN54ENV | 1 mL | Left leg |
| | ZN96GPN | ZN96GPN | ZN96GPN | 1 mL | Right leg |
| 3 | CN54ENV | CN54ENV | CN54ENV | 1 mL | Left leg |
| | ZN96GPN | ZN96GPN | ZN96GPN | 1 mL | Right leg |

| | Week | | | |
|---|---|---|---|---|
| Group | 13 | 16 | Volume | Site |
| 1 | Dilution buffer | Dilution buffer | 0.5 mL | Left leg |
| | gp140 | gp140 | | |
| 2 | MVAC-C | MVAC-C | 0.5 mL | Left leg |
| 3 | rgp 140/GLA | rgp 140/GLA | 0.4 mL | Left leg |
| | MVAC-C | MVAC-C | 0.5 mL | Right leg |

| | Week | | | | |
|---|---|---|---|---|---|
| Group | 19 | 22 | 25 | Volume | Site |
| 1 | Dilution | Dilution | Dilution | 0.4 mL | Left leg |
| | buffer | buffer | buffer | | |
| | gp140 | gp140 | gp140 | | |
| 2 | rgp | rgp | rgp | 0.4 mL | Left leg |
| | 140/GLA | 140/GLA | 140/GLA | | |

The site will be clipped and marked. Marking will be maintained.

### Justification of Route and Dosage Levels

The intramusular route of administration has been selected for this study as this route has been defined by the Sponsor as the route of clinical application.

The dose levels have been agreed with the Sponsor after examination of various data that are available. Dose level selection take into account the maximum tolerated dose in the test model and other factors such as anticipated therapeutic dose.

Clinically treatment will be every 4 weeks. In this preclinical study treatment will be shortened every 3 weeks and an additional rgp140 injection will be given to increase exposure. The DNA-C and MVAC-C have previously been tested and it is considered that there is little need to increase the frequency to these.

### In-Life Procedures, Observations, and Measurements

The in-life procedures, observations, and measurements listed below will be performed for all animals.

### Mortality/Moribundity Checks

Frequency: All animals will be checked early morning and as late as possible each day for viability.
Procedure: Any animal showing signs of severe debility or intoxication and if determined to be moribund or suffering excessively will be euthanised.

### Clinical Observations

### (a) Detailed Clinical Observations

Frequency: Weekly commencing the first week of treatment.
Procedure: Animals removed from the cage for examination.

### (b) Postdose Observations

Frequency: Dosing days—Regularly throughout the day of treatment. Non-dosing days—Once
Procedure: All the animals will be examined for reaction to treatment. The onset, intensity and duration of these signs will be recorded (if appropriate), particular attention being paid to the animals during and for the first hour after dosing.

### Dermal Scoring

Frequency: Dosing days—0 h (before dosing), 24 and 48 h after injection.
Procedure: Skin will be assessed for erythema and eschar formation, oedema formation, skin thickening, desquamation and any other reaction to treatment.

| Erythema and Eschar Formation | Grade |
|---|---|
| No erythema | 0 |
| Very slight erythema (barely perceptible) | 1 |
| Well defined erythema | 2 |
| Moderate to severe erythema | 3 |
| Severe erythema (beet redness) to slight | 4 |
| eschar formation (injuries in depth) | |

### Body Weights

| | | |
|---|---|---|
| Frequency: | Pretrial- | Once |
| | Dosing Period- | Weekly |
| | Recovery Period- | Weekly |

| | |
|---|---|
| Procedure: | Animals showing weight loss or deterioration in condition |
| | will be weighed more frequently as necessary. |

### Food Consumption

This will not be measured.

### Ophthalmic Examinations

Frequency: Pretrial—All animals once Dosing Period—All animals (after dosing) Weeks 16 and 25 Recovery Period—All recovery animals Week 29.
Procedure: The eyes will be examined using an indirect ophthalmoscope after the application of a mydriatic agent (1% Tropicamide, Mydriacyl®). The anterior, lenticular and fundic areas will be examined.

### Laboratory Evaluations

### Clinical Pathology

### (a) Sample Collection

Blood will be collected from an auricular artery using sterile syringes and needles after an appropriate time in a heating cabinet. Additional blood samples may be obtained (e.g., due to clotting of non-serum samples) if permissible sampling frequency and blood volume are not exceeded. After collection, samples will be transferred to the appropriate laboratory for processing.

Animals will not be fasted before blood sampling. Samples will be collected according to the following table.

### Samples for Clinical Pathology Evaluation

| | Time | | | Clinical |
|---|---|---|---|---|
| Group Nos. | Point | Haematology | Coagulation | Chemistry |
| 1-3 | Pretrial | X | X | X |
| 1-3 | Week 7 | X | X | X |
| 1-3 | Week 13 | X | X | X |
| 1-3 | Week 16 | X | X | X |
| 3 | Week 20 | X | X | X |
| 1-2 | Week 25 | X | X | X |
| 1-2 | Week 29 | X | X | X |
| Unscheduled | Before | X | X | X |
| euthanasia | euthanasia | | | |
| (when possible) | | | | |

X = sample to be collected;
— = not applicable

Any residual/retained clinical pathology samples will be discarded before issue of the Final Report.

### (b) Haematology

| | |
|---|---|
| Target Volume: | 0.5 mL |
| Anticoagulant: | EDTA |

Haematology Parameters

| | |
|---|---|
| Red blood cell count | White blood cell count |
| Haemoglobin | Neutrophils |
| Haematocrit | Lymphocytes |
| Mean cell volume | Monocytes |
| Mean cell haemoglobin | Eosinophils |
| concentration | Basophils |
| Mean cell haemoglobin | Large unstained cells |
| Reticulocytes | Other cells (as appropriate) |
| Reticulocyte count (absolute) | |
| Red blood cell distribution width | |
| Platelets | |
| Blood Smear (see \hat{ } below) | |

\hat{ } A bloodsmear will be prepared from each haematology specimen. Blood smears will be labelled, stained, stored and archived. The smears may be subsequently evaluated and this will be described in a protocol amendment with approval of the Study Director and Sponsor. A decision to evaluate the blood smears will be based upon the possibility that evaluation may further elucidate changes that have occurred in the numerical haematology parameters.

### (c) Coagulation

| | |
|---|---|
| Target Volume: | 0.9 mL |
| Anticoagulant: | 3.8% (w/v) trisodium citrate |
| Processing: | To plasma |

Coagulation Parameters

| | |
|---|---|
| Activated partial thromboplastin time | Prothrombin time |
| Fibrinogen | |

### (d) Clinical Chemistry

| | |
|---|---|
| Target Volume: | 1.5 mL |
| Anticoagulant: | Lithium Heparin |
| Processing: | To plasma |

Clinical Chemistry Parameters

| | |
|---|---|
| Urea | Total protein |
| Glucose | Albumin |
| Aspartate aminotransferase | Globulin |
| Alanine aminotransferase | Albumin/globulin ratio |
| Alkaline phosphatase | Cholesterol |
| Creatine phosphokinase | Creatinine |
| Lactate Dehydrogenase | Total bilirubin |
| Sodium | Calcium |
| Potassium | Inorganic phosphate |
| Chloride | |

### (e) Bone Marrow Smear Evaluation (Optional)

Bone marrow smears will be collected as described in the Tissue Collection and Preservation table (Section 16.5). Evaluation of stained smears may be added by amendment at the discretion of the Study Director in consultation with the pathologist and the Sponsor.

### Antibody Sample Collection, Processing, and Analysis

Blood will be collected from all animals or appropriate subsets from an auricular artery.
Time Points: Pretrial, Week 13 and 19 (before dosing) and at kill.
Target Volume: 2 mL or 15 mL at kill
Anticoagulant: None
Processing: To serum (at least 1500 g/5 min/4° C.)
Storage: −20° C.

It is not an absolute regulatory requirement that this preclinical study contain this type of analysis and the immune response is not monitored as part of safety assessment, but is being used to confirm immunogenicity.

### DNA Biodistribution

From Animals 8, 10, 29, 31, 15, 17, 36 and 38, tissues will be collected at necropsy and incorporation of DNA assessed using quantitative polymerase chain reaction. The method used has been validated under Charles River Study 312839.

Tissues to be examined are injection site, blood, testes/ovary, liver, heart, brain, spleen, kidney and lung.

### Terminal Procedures

Terminal procedures are summarised in the following table:

Terminal Procedures for Main Study and Recovery Animals

| | Number of | | Scheduled | Necropsy Procedures | | | | |
|---|---|---|---|---|---|---|---|---|
| Group | Animals | | Euthanasia | | Tissue | Organ | | |
| number | M | F | Week | Necropsy | Collection | Weights | Histology | Histopathology |
| 3 | 4 | 4 | 16 | X | X | X | Full Tissue | Full Tissue^{a} |
| 3 | 3 | 3 | 20 | X | X | X | Select | Select Tissue^{b} |
| | | | | | | | Tissue | |
| 1 | 4 | 4 | 25 | X | X | X | Full Tissue | Full Tissue^{a} |
| 2 | 4 | 4 | | X | X | X | | |
| 1 | 3 | 3 | 29 | X | X | X | Select | Select Tissue^{b} |
| 2 | 3 | 3 | | X | X | X | Tissue | |

| | | | | | |
|---|---|---|---|---|---|
| Unscheduled Deaths | X | X | — | Full Tissue | Full Tissue^{a} |

X = procedure to be conducted;
— = not applicable.
^{a}See Tissue Collection and Preservation table for listing of tissues.
^{b}Injection site, lumbar and inguinal lymph nodes.

### Unscheduled Deaths

If a main study or recovery animal dies on study, a necropsy will be conducted and specified tissues will be saved. If necessary, the animal will be refrigerated to minimise autolysis.

Main study or recovery animals may be euthanised for humane reasons as per Test Facility SOPs. The body weight will be recorded and samples for evaluation of clinical pathology parameters and antibody analysis, and PCR will be obtained if possible as specified in Section 0. These animals will undergo necropsy, and specified tissues will be retained. If necessary, the animal will be refrigerated to minimise autolysis.

### Scheduled Euthanasia

Main study and recovery animals surviving until scheduled euthanasia will be euthanised by an intravenous overdose of sodium pentabarbitone. The animals will be exsanguinated. Animals will not be fasted before their scheduled necropsy.

### Necropsy

Main study and recovery animals will be subjected to a complete necropsy examination, which will include evaluation of the carcass and musculoskeletal system; all external surfaces and orifices; cranial cavity and external surfaces of the brain; and thoracic, abdominal, and pelvic cavities with their associated organs and tissues. Necropsy examinations will be conducted by a trained technician and will consist of an external and internal examination and recording of observations for all animals. A veterinary pathologist will be available for consultation during normal working hours.

At the discretion of the necropsy supervising pathologist, images may be generated for illustration of or consultation on gross observations. Generation of such images will be documented and communicated to the Study Director. Images and associated documentation will be retained and archived.

### Organ Weights

The organs identified for weighing in the Tissues Collection and Preservation table will be weighed at necropsy for all scheduled euthanasia animals. Organ weights will not be recorded for animals found dead or euthanised in poor condition or in extremis. Paired organs will be reported together. Terminal body weights will be used for organ weight analysis.

### Tissue Collection and Preservation

Representative samples of the tissues identified in the Tissue Collection and Preservation table will be collected from all animals and preserved in 10% neutral buffered formalin, unless otherwise indicated. Additional tissue samples may be collected to elucidate abnormal findings.

### Tissue Collection and Preservation

| | | | Microscopic | |
|---|---|---|---|---|
| Tissue | Weigh | Collect | Evaluation | Comment |
| Administration site | — | X | X | Injection sites 1 and 2: Muscle around |
| | | | | the marked area will be collected as a |
| | | | | contingency. |
| Animal identification | — | X | — | — |
| Artery, aorta | — | X | X | From thoracic segment. |
| Bone marrow smear | — | X | — | One bone marrow smear will be |
| | | | | collected from the femur at scheduled |
| | | | | necropsies only (for possible |
| | | | | examination). Smears will not be |
| | | | | collected from animals that are found |
| | | | | dead. Bone marrow smears are |
| | | | | allowed to air dry and are not fixed in |
| | | | | formalin. |
| Bone marrow, femur | — | X | X | Collect with bone, femur |
| Bone marrow, | — | X | X | Collect with bone, sternum |
| sternum | | | | |
| Bone, femur with | — | X | X | Collect distal end to include femoral |
| articulating surface | | | | tibial joint |
| Bone, sternum | — | X | X | — |
| Brain | X | X | X | Forebrain, midbrain, cerebellum, and |
| | | | | medulla oblongata. |
| Cervix | — | X | X | Collect with uterus. |
| Epididymis | X | X | X | Separate weights and examination. |
| Eye | — | X | X | Separate examination; Preserve in |
| | | | | Davidson's fixative. |
| Gallbladder | — | X | X | |
| Gland, adrenal | X | X | X | Separate weights and examination. |
| Gland, lacrimal | — | X | X | Only 1 required for examination. |
| Gland, mammary | — | X | X | Collect with thoracic skin and include |
| | | | | nipple; mammary gland will be |
| | | | | examined in females only |
| Gland, parathyroid | — | X | X | Collect with thyroid: Examine only if |
| | | | | present in the routine section of |
| | | | | thyroid. |
| Gland, pituitary | X | X | X | — |
| Gland, prostate | X | X | X | — |
| Gland, salivary | — | X | X | Submandibular; Only 1 required for |
| | | | | examination. |
| Gland, seminal | — | X | X | |
| vesicle | | | | |
| Gland, thyroid | X | X | X | Separate weights and examination; |
| | | | | weight includes parathyroid |
| Gross | — | X | X | — |
| lesions/masses | | | | |
| Gut-associated | — | X | X | Collect with small intestine. |
| lymphoid tissue | | | | |
| Heart | X | X | X | — |
| Kidney | X | X | X | Separate weights and examination. |
| Large intestine, | — | X | X | |
| appendix | | | | |
| Large intestine, | — | X | X | — |
| cecum | | | | |
| Large intestine, colon | — | X | X | — |
| Large intestine, | — | X | X | — |
| rectum | | | | |
| Large intestine, | — | X | X | |
| sacculus rotundus | | | | |
| Liver | X | X | X | Drain gallbladder before weighing |
| Lung | X | X | X | Infuse with 10% neutral buffered |
| | | | | formalin after weighing. |
| Lymph node, | — | X | X | Only 1 required for examination. |
| mandibular | | | | |
| Lymph node, | — | X | X | — |
| mesenteric | | | | |
| Lymph node, lumbar | — | X | X | Identify left and right. |
| Lymph node, inguinal | — | X | X | Identify left and right. |
| Muscle, skeletal | — | X | X | From thigh |
| Nerve, optic | — | X | X | Preserve in Davidson's fixative; |
| | | | | Examine only if present in the routine |
| | | | | section of the eye. |
| Nerve, sciatic | — | X | X | Only 1 required for examination. |
| Oesophagus | — | X | X | — |
| Ovary | X | X | X | Separate weights and examination. |
| Oviduct | — | X | X | Only 1 required for examination. |
| | | | | Collect with uterus. |
| Pancreas | — | X | X | — |
| Skin | — | X | X | Collect with mammary gland. |
| Small intestine, | — | X | X | — |
| duodenum | | | | |
| Small intestine, ileum | — | X | X | — |
| Small intestine, | — | X | X | — |
| jejunum | | | | |
| Spinal cord | — | X | X | Cervical, thoracic, lumbar. |
| Spleen | X | X | X | — |
| Stomach | — | X | X | Fundus and pylorus |
| Testis | X | X | X | Separate weights and examination; |
| | | | | Preserve in Modified Davidson's |
| | | | | fixative. |
| Thymus | X | X | X | — |
| Tongue | — | X | X | — |
| Trachea | — | X | X | — |
| Ureter | — | X | X | Only 1 required for examination. |
| Urinary bladder | — | X | X | — |
| Uterus | X | X | X | — |
| Vagina | — | X | X | See below |

X = procedure to be conducted;
— = not applicable.

### Rabbit Vaginal and Vestibular Mucosal Secretion Sample Collection

### (a) Equipment

Micropipettor, single channel, 200-1000 μL (Gilson, or equivalent)
Ice bucket
Scissors

### (b) Materials

Disposable, sterile micropipettor tips 1000 μL
Gloves, latex or equivalent
Weck-Cel® surgical spears (Medtronic USA, 0008680)—to be supplied by Sponsor
Spin-X tubes (Costar 8160)

### (c) Reagents

Extraction buffer. Store at 4° C.; the expiry date will be detailed on the buffer container.
   Note: 100 mL extraction buffer contains 1 mL 100× protease inhibitor cocktail I, 20 μL 10% sodium azide solution, and 1.5 g NaCl; made up to a final volume of 100 mL with 1× sterile Dulbecco's phosphate buffered saline.

### (d) Procedure

Prepare Spin-X tubes by placing 300 μL of extraction buffer into the upper chamber using the micropipettor with sterile tip. Store prepared tubes on ice or at 4° C. until use. Tubes may be prepared in advance and stored at 4° C. until the expiry date marked on the extraction buffer container.

Place a Weck-Cel spear on the mucosal surface of the vagina, and another on the mucosal surface of the vestibule, for 2 minutes to soak up any secretion. Do not apply the spears to the exact same areas that will be processed for histopathology, as the spears might disturb the mucosal surface.

Remove the Weck-Cel spears and place each into the top chamber of a separate Spin-X tube containing 300 μL of extraction buffer.

Using a pair of scissors carefully cut each Weck-Cel spear at the base of the spear head, discarding the handle. Close the lid of the top chamber of the Spin-X tubes and place at ≦−55° C.

### (e) Additional Information

The time from sampling to storage should not exceed 2 hours.

### Histology and Histopathology

### Histology

Tissues in the Tissue Collection and Preservation table from animals identified in the Terminal Procedures table will be embedded in paraffin, sectioned, mounted on glass slides, and stained with haematoxylin and eosin.

### Histopathology

Histopathological evaluation will be performed by a veterinary pathologist with training and experience in laboratory animal pathology. Tissues identified as target tissues will be examined from animals identified in the Terminal Procedures table, by protocol amendment. Any additional stains or evaluations, if deemed necessary by the pathologist, will be added by protocol amendment following discussion with the Study Director and in consultation with the Sponsor.

At the discretion of the study pathologist and after acknowledgement by the study director, images may be captured for consultation purposes.

### Pathology Peer Review

A pathology peer review, as per the appropriate SOP of the Pathology Department, will be conducted by a second pathologist.

### Computerised Systems

The following critical computerised systems will be used in the study. Any additional critical computerised systems used during the course of the study will be added by protocol amendment. The actual critical computerised systems used will be specified in the Final Report. Data for parameters not required by protocol, which are automatically generated by analytical devices used will be retained on file but not reported. Statistical analysis results that are generated by the program but are not required by protocol and/or are not scientifically relevant will be retained on file but will not be included in the tabulations.

### Proposed Critical Computerised Systems

| System Name | Description of Data Collected and/or Analysed |
|---|---|
| Dispense | Dose Formulation |
| Provantis | In-life data collection and reporting |
| Nautilus 2003 | Clinical Pathology Laboratory Information |
| | Management System (LIMS) |
| Atlas 2002 | Chromatography Data Collection and Processing |
| | (Formulation Analysis) |
| PLACES 2000 | Histopathology/Organ Weights |

### Statistical Analysis

Unless otherwise stated, all statistical tests will be two-sided and performed at the 5% significance level using in-house software. Males and females will be analysed separately.

Pairwise comparisons will only be performed against the control group (Group 1). The following pairwise comparisons will be performed:
Control Group vs Group 2
Control Group vs Group 3

Body weight, hematology, coagulation and clinical chemistry will be analysed for homogeneity of variance using the ‘F-Max’ test. If the group variances appear homogeneous, a parametric ANOVA will be used and pairwise comparisons will be made using Fisher's F protected LSD method via Student's t test ie pairwise comparisons will be made only if the overall F-test is significant. If the variances are heterogeneous, log or square root transformations will be used in an attempt to stabilise the variances. If the variances remain heterogeneous, then a Kruskal-Wallis non-parametric ANOVA will be used and pairwise comparisons will be made using chi squared protection (via z tests, the non-parametric equivalent of Student's t test).

In circumstances where it is not possible to perform the F Max test due to zero standard deviation in at least one group, the non-parametric ANOVA results will be reported.

Organ weights will be analysed using ANOVA as above and by analysis of covariance (ANCOVA) using terminal kill body weight as covariate. In addition, organ weights as a percentage of terminal body weight will be analysed using ANOVA as above as an exploratory analysis. The results of this analysis will only be presented in the study report if required to aid interpretation.

In circumstances where the variances in the ANCOVA remain heterogeneous following log or square root transformations, the data will be subjected to a rank transformation prior to analysis. Where it is not possible to perform the F-Max test due to the small sample size (i.e. less than 3 animals in any group), the untransformed parametric ANCOVA results will be reported.

In the ANOVA and ANCOVA summary tables, the results of the analysis will be reported indicating the level of statistical significance (p<0.05, p<0.01 and p<0.001) of each pairwise comparison.

Actual p-values will not be reported in the summary tables for these analyses.

### Results

### Immunisation Schedule

| | Group/Treatment/Dosage | | |
|---|---|---|---|
| Week of Treatment | Group 1 | Group 2 | Group 3 |
| 1 | Vehicle (phosphate buffered saline) | DNA-C | DNA-C |
| | | 4 mg | 4 mg |
| 4 | Vehicle (phosphate buffered saline) | DNA-C | DNA-C |
| | | 4 mg | 4 mg |
| 7 | Vehicle (phosphate buffered saline) | DNA-C | DNA-C |
| | | 4 mg | 4 mg |
| 13 | Vehicle (gp 140 dilution buffer) | MVA-C | MVA-C/gp 140/GLA |
| | | 1.3 × 10⁸ TCID₅₀ | 1.3 × 10⁸ TCID₅₀/100 μg/5 μg |
| 16 | Vehicle (gp 140 dilution buffer) | MVA-C | MVA-C/gp 140/GLA |
| | | 1.3 × 10⁸ TCID₅₀ | 1.3 × 10⁸ TCID₅₀/100 μg/5 μg |
| 19 | Vehicle (gp 140 dilution buffer) | gp 140/GLA | — |
| | | 100 μg/5 μg | |
| 22 | Vehicle (gp 140 dilution buffer) | gp 140/GLA | — |
| | | 100 μg/5 μg | |
| 25 | Vehicle (gp 140 dilution buffer) | gp 140/GLA | — |
| | | 100 μg/5 μg | |

| | Pre-Trial | Week 18 | SEM | Week 21 | Week 24 | Week 27 | Terminal |
|---|---|---|---|---|---|---|---|
| Group 1 | 0 | 0 | 0 | 0 | 0 | 0 | 0 |
| Group 2 | 0 | 405121.352 | 35706.269 | 793033.836 | 1095834.11 | 1137791.81 | |
| Group 3 | 0 | 1097607.18 | 215816.103 | | | | 1356339.22 [symbol] |

See FIG. 17. [symbol]
indicates data missing or illegible when filed

| | Pre-Trial | SEM | Week 18 | SEM | Week 27 | SEM | |
|---|---|---|---|---|---|---|---|
| Group 1 | 0 | 0 | 0 | 0 | 0 | 0 | 0 |
| Group 2 | 0 | 0 | | | 1137791.81 | 514861.392 | 514861.392 |
| Group 3 | 0 | 0 | 1097607.18 | 215816.103 | | | 215816.103 |

See FIG. 18.
See also FIG. 19.

Analysis of the (presumed) peak antibody responses measured 2 weeks after the last immunisation in each group of 13-14 rabbits strongly corroborated what had been seen in the mouse and confirmed that the accelerated regimen did not result in attenuation of the antibody response, but rather the very similar (and possibly maximal) response of ˜1500 μg/ml of systemic CN54rgp140-specific IgG could be achieved in a shorter time, demonstrating the advantage of the accelerated regimen at least in respect of this immune response.

1. A method of raising an immune response in an individual against human immunodeficiency virus (HIV), the method comprising:
(a) administering to the individual DNA or SFV encoding an HIV Env protein and optionally nucleic acid encoding one, any two or all three of the HIV Gag, Pol and Nef proteins;
(b) subsequently administering to the individual a viral vector encoding an HIV Env protein and optionally a viral vector encoding one, any two or all three of the HIV Gag, Pol and Nef proteins; and
(c) administering to the individual an oligomeric HIV Env protein; and an adjuvant.

2. A method according to claim 1 wherein in step (a) the DNA is naked DNA.

3. A method according to claim 1 wherein in step (a) the DNA is comprised in one or more adenoviral vectors.

4. A method according to claim 1 wherein in step (a) nucleic acid encoding each of HIV Env, Gag, Pol and Nef proteins is administered to the individual.

5. A method according to claim 2 wherein in step (a) naked DNA is administered two or more times to the individual with at least three weeks between each administration.

6. A method according to claim 2 wherein at least 1 mg of naked DNA is administered to the individual in each administration.

7. A method according to claim 1 wherein in step (b) the viral vector is a pox virus vector, such as a vaccinia vector.

8. A method according to claim 1 wherein in step (b) the viral vector is an adenoviral vector.

9. A method according to claim 1 wherein in step (b) viral vector encoding each of HIV Env, Gag, Pol and Nef is administered to the individual.

10. A method according to claim 1 wherein in step (b) viral vector is administered two or more times with around four weeks between doses.

11. A method according to claim 1 wherein in step (c) the oligomeric HIV Env protein is oligomeric gp140.

12. A method according to claim 1 wherein in step (c) the adjuvant is selected from the group consisting of glucopyranosyl lipid A (such as GLA-AF) and AS02.

13. A method according to claim 1 wherein in each of steps (a), (b) and (c) HIV Env protein, and when it is present, HIV Gag, Pol and Nef protein has substantially the same amino acid sequence.

14. A method according to claim 1 wherein steps (b) and (c) are carried out simultaneously.

15. A method according to claim 1 wherein step (c) is carried out around four weeks after step (b).

16.-21. (canceled)

22. A composition comprising an oligomeric HIV Env protein and a GLA adjuvant.

23. A kit of parts comprising an oligomeric HIV Env protein; and an adjuvant.

24. A kit of parts according to claim 23 wherein the adjuvant is GLA.

25. A kit of parts according to claim 23 further comprising a viral vector encoding an HIV Env protein and optionally viral vector encoding one, any two or all three of the HIV Gag, Pol and Nef proteins.

26. A kit of parts according to claim 23 further comprising DNA or SFV encoding an HIV Env protein and optionally nucleic acid encoding one, any two or all three of the HIV Gag, Pol and Nef protein.


SEQUENCE LISTING (12 sequences)

SEQ ID NO: 1 - protein, 8 aa, artificial
  HIV - CN54gp140 N terminal species 1
SQEIHARF

SEQ ID NO: 2 - protein, 8 aa, artificial
  HIV - CN54gp140 N terminal species 2
GARSGNLW

SEQ ID NO: 3 - protein, 634 aa, artificial
  HIV - CN54gp140
GNLWVTVYYGVPVWKGATTTLFCASDAKAYDTEVHNVWATHACVPADPNPQEMVLENVTENFNMWKNEMVNQMQEDVISLWDQSLKPCVKLTPLCVTLECRNVSSNSNDTYHETYHESMKEMKNCSFNATTVVRDRKQTVYALFYRLDIVPLTKKNYSENSSEYYRLINCNTSAITQACPKVTFDPIPIHYCTPAGYAILKCNDKIFNGTGPCHNVSTVQCTHGIKPVVSTQLLLNGSLAEGEIIIRSENLTNNVKTIIVHLNQSVEIVCTRPGNNTRKSIRIGPGQTFYATGDIIGDIRQAHCNISEDKWNETLQRVSKKLAEHFQNKTIKFASSSGGDLEVTTHSFNCRGEFFYCNTSGLFNGAYTPNGTKSNSSSIITIPCRIKQIINMWQEVGRAMYAPPIKGNITCKSNITGLLLVRDGGTEPNDTETFRPGGGDMRNNWRSELYKYKVVEIKPLGVAPTTTKRRVVEREKRAVGIGAVFLGFLGVAGSTMGAASITLTVQARQLLSGIVQQQSNLLRAIEAQQHLLQLTVWGIKQLQTRVLAIERYLKDQQLLGIWGCSGKLICTTAVPWNSSWSNKSQKEIWDNMTWMQWDKEISNYTNTVYRLLEESQNQQERNEKDLLALDSWKN

SEQ ID NO: 4 - DNA, 13563 bp, artificial
  pLZAW1gp120C/gagpolnefC-14 transfer vector
gccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgtaattaactagctacccgggagaaaaattatttttgacaccagaccaactggtaatggtagcgaccggcgctcagctgtaattccgccgatactgacgggctccaggagtcgtcgccaccaatccccatatggaaaccgtcgatattcagccatgtgccttcttccgcgtgcagcagatggcgatggctggtttccatcagttgctgttgactgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttcttgcggccctaatccgagccagtttacccgctctgctacctgcgccagctggcagttcaggccaatccgcgccggatgcggtgtatcgctcgccacttcaacatcaacggtaatcgccatttgaccactaccatcaatccggtaggttttccggctgataaataaggttttcccctgatgctgccacgcgtgagcggtcgtaatcagcaccgcatcagcaagtgtatctgccgtgcactgcaacaacgctgcttcggcctggtaatggcccgccgccttccagcgttcgacccaggcgttagggtcaatgcgggtcgcttcacttacgccaatgtcgttatccagcggtgcacgggtgaactgatcgcgcagcggcgtcagcagttgttttttatcgccaatccacatctgtgaaagaaagcctgactggcggttaaattgccaacgcttattacccagctcgatgcaaaaatccatttcgctggtggtcagatgcgggatggcgtgggacgcggcggggagcgtcacactgaggttttccgccagacgccactgctgccaggcgctgatgtgcccggcttctgaccatgcggtcgcgttcggttgcactacgcgtactgtgagccagagttgcccggcgctctccggctgcggtagttcaggcagttcaatcaactgtttaccttgtggagcgacatccagaggcacttcaccgcttgccagcggcttaccatccagcgccaccatccagtgcaggagctcgttatcgctatgacggaacaggtattcgctggtcacttcgatggtttgcccggataaacggaactggaaaaactgctgctggtgttttgcttccgtcagcgctggatgcggcgtgcggtcggcaaagaccagaccgttcatacagaactggcgatcgttcggcgtatcgccaaaatcaccgccgtaagccgaccacgggttgccgttttcatcatatttaatcagcgactgatccacccagtcccagacgaagccgccctgtaaacggggatactgacgaaacgcctgccagtatttagcgaaaccgccaagactgttacccatcgcgtgggcgtattcgcaaaggatcagcgggcgcgtctctccaggtagcgaaagccattttttgatggaccatttcggcacagccgggaagggctggtcttcatccacgcgcgcgtacatcgggcaaataatatcggtggccgtggtgtcggctccgccgccttcatactgcaccgggcgggaaggatcgacagatttgatccagcgatacagcgcgtcgtgattagcgccgtggcctgattcattccccagcgaccagatgatcacactcgggtgattacgatcgcgctgcaccattcgcgttacgcgttcgctcatcgccggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgcctcgaatcagcaacggcttgccgttcagcagcagcagaccattttcaatccgcacctcgcggaaaccgacatcgcaggcttctgcttcaatcagcgtgccgtcggcggtgtgcagttcaaccaccgcacgatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacgcaactcgccgcacatctgaacttcagcctccagtacagcgcggctgaaatcatcattaaagcgagtggcaacatggaaatcgctgatttgtgtagtcggtttatgcagcaacgagacgtcacggaaaatgccgctcatccgccacatatcctgatcttccagataactgccgtcactccaacgcagcaccatcaccgcgaggcggttttctccggcgcgtaaaaatgcgctcaggtcaaattcagacggcaaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggttacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacgggatcgcgcttgagcagctccttgctggtgtccagaccaatgcctcccagaccggcaacgaaaatcacgttcttgttggtcaaagtaaacgacatttttagagagaactaacacaaccagcaataaaactgaacctactttatcatttttttattcggatcctagcttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgggtaccaaggcgcgatcgcattttctaacgtgatggatatattaaagtcgaataaagtgaacaataattaattctttattgtcatcatgtaattaactagctacccggaataaaaattccgggagatctctcgagagatctttatcacctcttctccctctccaccaccctcctcttggtggtggtgggggccacgcccaggggcttgatctccaccaccttgtacttgtacagctcgctcctccagttgttcctcatgtcgccgccgccgggcctgaaggtctcggtgtcgttgggctcggtgccgccgtccctcaccagcagcaggccggtgatgttgctcttgcaggtgatgttgcccttgatgggaggggcgtacatggccctgcccacctcctgccacatgttgatgatctgcttgatcctgcaggggatggtgatgatgctgctgctgttgctcttggtgccgttgggggtgtaggcgccgttgaacaggccgctggtgttgcagtagaagaactcgcccctgcagttgaagctgtgggtggtcacctccaggtcgccgccgctgctgctggcgaacttgatggtcttgttctggaagtgctcggcaagcttcttgctcaccctctgcagggtctcgttccacttgtcctcgctgatgttgcagtgggcctgcctgatgtcgccgatgatgtcgccggtggcgtagaaggtctggccggggccgatcctgatgctcttcctggtgttgttgccgggcctggtgcacacgatctccacgctctggttcaggtgcacgatgatggttttcacgttgttggtcaggttctcgctcctgatgatgatctcgccctcggccaggctgccgttcagcagcagctgggtgctcaccacgggcttgatgccgtgggtgcactgcacggtgctcacgttgtggcaggggccggtgccgttgaagatcttgtcgttgcacttcaggatggcgtagccggcgggggtgcagtagtggatggggatggggtcgaaggtcaccttggggcaggcctgggtgatggcgctggtgttgcagttgatcagcctgtagtactcgctgctgttctcgctgtagttcttcttggtcaggggcacgatgtccagcctgtagaacagggcgtacacggtctgcttcctgtccctcaccacggtggtggcgttgaagctgcagttcttcatctccttcatgctctcgtggtaggtctcgtggtaggtgtcgttgctgttgctgctcacgttcctgcactccagggtcacgcacaggggggtcagcttcacgcagggcttcaggctctggtcccacaggctgatgacgtcctcctgcatctggttcaccatctcgttcttccacatgttgaagttctcggtcacgttctccagcaccatctcctgggggttggggtcggcgggcacgcaggcgtgggtggcccacacgttgtgcacctcggtgtcgtaggccttggcgtcgctggcgcagaacagggtggtggtggcgcccttccacacgggcacgccgtagtacacggtcacccacaggttgcccacggcctgggcctggggcagcagcagcagcagcagcagcagcagcagcttggccctgtccatgctcgagcttatttatattccaaaaaaaaaaaataaaatttcaatttttaagcttgtcgacaaaaattgaaattttattttttttttttggaatataaatagactcgagcatggccgccagggccagcatcctgaggggcggcaagctggacaagtgggagaagatcaggctgaggcccggcggcaagaagcactacatgctgaagcacctggtgtgggccagcagggagctggagaggttcgccctgaaccccggcctgctggagaccagcgagggctgcaagcagatcatgaagcagctgcagagcgccctgcagaccggcaccgaggagctgaggagcctgttcaacaccgtggccaccccctactgcgtgcacaccgagatcgacgtgagggacaccagggaggccctggacaagatcgaggaggagcagaacaagatccagcagaagacccagcaggccaaggaggccgacggcaaggtgagccagaactaccccatcgtgcagaacctgcagggccagatggtgcaccagcccatcagccccaggaccctgaatgcatgggtgaaggtggtggaggagaaggccttcagccccgaggtgatccccatgttcagcgccctgagcgagggcgccacccctcaggacctgaacaccatgctgaacaccgtgggcggccaccaggccgccatgcagatcctgaaggacaccatcaacgaggaggccgccgagtgggacaggctgcaccccgtgcacgccggccccatcgcccccggccagatgagggagcccaggggcagcgacatcgccggcaccaccagcaacctgcaggagcagatcgcctggatgaccagcaacccacccgtgcccgtgggcgacatctacaagaggtggatcatcctgggtttaaacaagatcgtgaggatgtacagccccaccagcatcctggacatcaagcagggccccaaggagcccttcagggactacgtggacaggttcttcaagaccctgagggccgagcaggccacccagggcgtgaagaactggatgaccgacaccctgctggtgcagaacgccaaccccgactgcaagaccatcctgagggccctgggccccggcgccagcatcgaggagatgatgaccgcctgccagggcgtgggcggccccagccacaaggccaaggtgctggccgaggccatgagccagaccaacagcgccatcctgatgcagaggagcaacttcaagggcagcaagaggatcgtgaagtgcttcaactgcggcaaggagggccacatcgccaggaactgcagggcccccaggaagaagggctgctggaagtgcggcaaggagggccaccagatgaaggactgcaccgagaggcaggccaacttcctgggcaagatctggcccagccacaagggcggccccggcaacttcctgcagaacaggcccgagcccaccgccccccccgaggagagcttcaggttcgaggaggagaccaccacccccagccagaagcaggagcccatcgacaaggagctgtaccccctgaccagcctgaagagcctgttcggcaacgaccccagcagccaggaattcttcagggagaacctggccctgccccagggcagggccagggagttcagcagcgagcagaccagggccaacagccccaccaggggcgagctgcaggtgtggggcagggacaacaacagcatcagcgaggccggcgccaacaggcagggcaccatcagcttcaacttcccccagatcaccctgtggcagaggcccctggtgaccatcaagatcggcggccagctgaaggaggccctgctgaacaccggcgccggcgacaccgtgctggaggacctgaacctgcccggcaagtggaagcccaagatgatcggcggcatcggcggcttcatcaaggtgaggcagtacgagcagatccccatcgagatctgcggccacaaggccatcggcaccgtgctggtgggccccacccccgtgaacatcatcggcaggaacctgctgacccagctgggctgcaccctgaacttccccatcagccccatcgagaccgtgcccgtgaagctgaagcccggcatggacggccccaaggtgaagcagtggcccctgaccgaggagaagatcaaggccctgaccgccatctgcgacgagatggagaaggagggcaagatcaccaagatcggccccgagaacccctacaacacccccatcttcgccatcaagaagaaggacagcaccaagtggaggaagctggtggacttcagggagctgaacaagaggacccaggacttctgggaggtgcagctgggcatcccccaccccgccggcctgaagaagaagaagagcgtgaccgtgctggacgtgggcgacgcctacttcagcatccccctgtacgaggacttcaggaagtacaccgccttcaccatccccagcaggaacaacgagacccccggcatcagctaccagtacaacgtgctgccccagggctggaagggcagcctcgccatcttccagagcagcatgaccatcgaggagctgatctacagcaagaagaggcaggagatcctggacctgtgggtgtaccacacccagggctacttccccgactggcacaactacacccccggccccggcgtgaggttccccctgaccttcggctggtgcttcaagctggtgcccgtggaccccagggaggtggaggaggccaacgagggcgaggacaactgcctgctgcaccccgtgtgccagcacggcatggaggacgaccacagggaggtgctgaagtggaagttcgacagccagctggcccacaggcacagggccagggagctgcaccccgagttctacaaggactgcatgggcggcaagtggagcaagagcagcatcgtgggctggcccgccatcagggagaggatgaggaggaccgagcccgccgccgacggcgtgggcgccgtgagcagggacctggagaagcacggcgccatcaccagcagcaacaccgccgccaccaacgaggactgcgcctggctggaggcccaggaggagggcgaggtgggcttccccgtgaggccccaggtgcccctgaggcccatgacctacaagggcgccgtggacctgagcttcttcctgaaggagaagggcggcctggagggcctgaggcagcacctgctgaggtggggcttcaccacccccgacaagaagcaccagaaggagccccccttcctgtggatgggctacgagctgcaccccgacaagtggaccgtgcagcccacccagctgcccgagaaggatagctggaccgtgaacgacatccagaagctggtgggcaagctgaactgggccagccagatctaccccggcatcaaggtgaggcagctgtgcaagctgctgaggggcgccaaggccctgaccgacatcgtgcccctgaccgaggaggccgagctggagctggccgagaacagggagatcctgaaggagcccgtgcacggcgtgtactacgaccccagcaaggacctgatcgccgagatccagaagcagggccaggagcagtggacctaccagatctaccaggagcccttcaagaacctgaagaccggcaagtacgccaagatgaggaccgcccacaccaacgacgtgaagcagctgaccgaggccgtgcagaagatcgccatggagggcatcgtgatctggggcaagacccccaagttcaggctgcccatccagaaggagacctgggagacctggtggaccgactactggcaggccacctggatccccgagtgggagttcgtgaacacccctcccctggtgaagctgtggtatcagctggagaaggaccccatcgtgggcgtggagaccttctacgtggacggcgccgccaacagggagaccaagatcggcaaggccggctacgtgaccgacaggggcaggaagaagatcgtgagcctgaccgagaccaccaaccagaagaccgagctgcaggccatctgcatcgccctgcaggacagcggcagcgaggtgaacatcgtgaccgacagccagtacgccctgggcatcatccaggcccagcccgacaagagcgagagcgagctggtgaaccagatcatcgagcagctgatgaagaaggagagggtgtacctgagctgggtgcccgcccacaagggcatcggcggcaacgagcaggtggacaagctggtgagcagcggcatcaggaaggtgctgaagaccctggagcccttcaggaagcagaaccccggcatcgtgatctaccagtacatggacgacctgtacgtgggcagcgacctggagatcggccagcacaggaccaagtaaagatctctcgaggagctcaagcgggcggatcccccgggctgcaggaattcgatcgcgccaaatttaaatgatcctgatcctttttctgggtaagtaatacgtcaaggagaaaacgaaacgatctgtagttagcggccgcctaattaactaatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcaggggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgag

SEQ ID NO: 5 - DNA, 7347 bp, artificial
  HIV - MVA-C after homologous recombination
aagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgggtaccaaggcgcgatcgcattttctaacgtgatggatatattaaagtcgaataaagtgaacaataattaattctttattgtcatcatgtaattaactagctacccggaataaaaattccgggagatctctcgagagatctttatcacctcttctccctctccaccaccctcctcttggtggtggtgggggccacgcccaggggcttgatctccaccaccttgtacttgtacagctcgctcctccagttgttcctcatgtcgccgccgccgggcctgaaggtctcggtgtcgttgggctcggtgccgccgtccctcaccagcagcaggccggtgatgttgctcttgcaggtgatgttgcccttgatgggaggggcgtacatggccctgcccacctcctgccacatgttgatgatctgcttgatcctgcaggggatggtgatgatgctgctgctgttgctcttggtgccgttgggggtgtaggcgccgttgaacaggccgctggtgttgcagtagaagaactcgcccctgcagttgaagctgtgggtggtcacctccaggtcgccgccgctgctgctggcgaacttgatggtcttgttctggaagtgctcggcaagcttcttgctcaccctctgcagggtctcgttccacttgtcctcgctgatgttgcagtgggcctgcctgatgtcgccgatgatgtcgccggtggcgtagaaggtctggccggggccgatcctgatgctcttcctggtgttgttgccgggcctggtgcacacgatctccacgctctggttcaggtgcacgatgatggttttcacgttgttggtcaggttctcgctcctgatgatgatctcgccctcggccaggctgccgttcagcagcagctgggtgctcaccacgggcttgatgccgtgggtgcactgcacggtgctcacgttgtggcaggggccggtgccgttgaagatcttgtcgttgcacttcaggatggcgtagccggcgggggtgcagtagtggatggggatggggtcgaaggtcaccttggggcaggcctgggtgatggcgctggtgttgcagttgatcagcctgtagtactcgctgctgttctcgctgtagttcttcttggtcaggggcacgatgtccagcctgtagaacagggcgtacacggtctgcttcctgtccctcaccacggtggtggcgttgaagctgcagttcttcatctccttcatgctctcgtggtaggtctcgtggtaggtgtcgttgctgttgctgctcacgttcctgcactccagggtcacgcacaggggggtcagcttcacgcagggcttcaggctctggtcccacaggctgatgacgtcctcctgcatctggttcaccatctcgttcttccacatgttgaagttctcggtcacgttctccagcaccatctcctgggggttggggtcggcgggcacgcaggcgtgggtggcccacacgttgtgcacctcggtgtcgtaggccttggcgtcgctggcgcagaacagggtggtggtggcgcccttccacacgggcacgccgtagtacacggtcacccacaggttgcccacggcctgggcctggggcagcagcagcagcagcagcagcagcagcagcttggccctgtccatgctcgagcttatttatattccaaaaaaaaaaaataaaatttcaatttttaagcttgtcgacaaaaattgaaattttattttttttttttggaatataaatagactcgagcatggccgccagggccagcatcctgaggggcggcaagctggacaagtgggagaagatcaggctgaggcccggcggcaagaagcactacatgctgaagcacctggtgtgggccagcagggagctggagaggttcgccctgaaccccggcctgctggagaccagcgagggctgcaagcagatcatgaagcagctgcagagcgccctgcagaccggcaccgaggagctgaggagcctgttcaacaccgtggccaccccctactgcgtgcacaccgagatcgacgtgagggacaccagggaggccctggacaagatcgaggaggagcagaacaagatccagcagaagacccagcaggccaaggaggccgacggcaaggtgagccagaactaccccatcgtgcagaacctgcagggccagatggtgcaccagcccatcagccccaggaccctgaatgcatgggtgaaggtggtggaggagaaggccttcagccccgaggtgatccccatgttcagcgccctgagcgagggcgccacccctcaggacctgaacaccatgctgaacaccgtgggcggccaccaggccgccatgcagatcctgaaggacaccatcaacgaggaggccgccgagtgggacaggctgcaccccgtgcacgccggccccatcgcccccggccagatgagggagcccaggggcagcgacatcgccggcaccaccagcaacctgcaggagcagatcgcctggatgaccagcaacccacccgtgcccgtgggcgacatctacaagaggtggatcatcctgggtttaaacaagatcgtgaggatgtacagccccaccagcatcctggacatcaagcagggccccaaggagcccttcagggactacgtggacaggttcttcaagaccctgagggccgagcaggccacccagggcgtgaagaactggatgaccgacaccctgctggtgcagaacgccaaccccgactgcaagaccatcctgagggccctgggccccggcgccagcatcgaggagatgatgaccgcctgccagggcgtgggcggccccagccacaaggccaaggtgctggccgaggccatgagccagaccaacagcgccatcctgatgcagaggagcaacttcaagggcagcaagaggatcgtgaagtgcttcaactgcggcaaggagggccacatcgccaggaactgcagggcccccaggaagaagggctgctggaagtgcggcaaggagggccaccagatgaaggactgcaccgagaggcaggccaacttcctgggcaagatctggcccagccacaagggcggccccggcaacttcctgcagaacaggcccgagcccaccgccccccccgaggagagcttcaggttcgaggaggagaccaccacccccagccagaagcaggagcccatcgacaaggagctgtaccccctgaccagcctgaagagcctgttcggcaacgaccccagcagccaggaattcttcagggagaacctggccctgccccagggcagggccagggagttcagcagcgagcagaccagggccaacagccccaccaggggcgagctgcaggtgtggggcagggacaacaacagcatcagcgaggccggcgccaacaggcagggcaccatcagcttcaacttcccccagatcaccctgtggcagaggcccctggtgaccatcaagatcggcggccagctgaaggaggccctgctgaacaccggcgccggcgacaccgtgctggaggacctgaacctgcccggcaagtggaagcccaagatgatcggcggcatcggcggcttcatcaaggtgaggcagtacgagcagatccccatcgagatctgcggccacaaggccatcggcaccgtgctggtgggccccacccccgtgaacatcatcggcaggaacctgctgacccagctgggctgcaccctgaacttccccatcagccccatcgagaccgtgcccgtgaagctgaagcccggcatggacggccccaaggtgaagcagtggcccctgaccgaggagaagatcaaggccctgaccgccatctgcgacgagatggagaaggagggcaagatcaccaagatcggccccgagaacccctacaacacccccatcttcgccatcaagaagaaggacagcaccaagtggaggaagctggtggacttcagggagctgaacaagaggacccaggacttctgggaggtgcagctgggcatcccccaccccgccggcctgaagaagaagaagagcgtgaccgtgctggacgtgggcgacgcctacttcagcatccccctgtacgaggacttcaggaagtacaccgccttcaccatccccagcaggaacaacgagacccccggcatcagctaccagtacaacgtgctgccccagggctggaagggcagcctcgccatcttccagagcagcatgaccatcgaggagctgatctacagcaagaagaggcaggagatcctggacctgtgggtgtaccacacccagggctacttccccgactggcacaactacacccccggccccggcgtgaggttccccctgaccttcggctggtgcttcaagctggtgcccgtggaccccagggaggtggaggaggccaacgagggcgaggacaactgcctgctgcaccccgtgtgccagcacggcatggaggacgaccacagggaggtgctgaagtggaagttcgacagccagctggcccacaggcacagggccagggagctgcaccccgagttctacaaggactgcatgggcggcaagtggagcaagagcagcatcgtgggctggcccgccatcagggagaggatgaggaggaccgagcccgccgccgacggcgtgggcgccgtgagcagggacctggagaagcacggcgccatcaccagcagcaacaccgccgccaccaacgaggactgcgcctggctggaggcccaggaggagggcgaggtgggcttccccgtgaggccccaggtgcccctgaggcccatgacctacaagggcgccgtggacctgagcttcttcctgaaggagaagggcggcctggagggcctgaggcagcacctgctgaggtggggcttcaccacccccgacaagaagcaccagaaggagccccccttcctgtggatgggctacgagctgcaccccgacaagtggaccgtgcagcccacccagctgcccgagaaggatagctggaccgtgaacgacatccagaagctggtgggcaagctgaactgggccagccagatctaccccggcatcaaggtgaggcagctgtgcaagctgctgaggggcgccaaggccctgaccgacatcgtgcccctgaccgaggaggccgagctggagctggccgagaacagggagatcctgaaggagcccgtgcacggcgtgtactacgaccccagcaaggacctgatcgccgagatccagaagcagggccaggagcagtggacctaccagatctaccaggagcccttcaagaacctgaagaccggcaagtacgccaagatgaggaccgcccacaccaacgacgtgaagcagctgaccgaggccgtgcagaagatcgccatggagggcatcgtgatctggggcaagacccccaagttcaggctgcccatccagaaggagacctgggagacctggtggaccgactactggcaggccacctggatccccgagtgggagttcgtgaacacccctcccctggtgaagctgtggtatcagctggagaaggaccccatcgtgggcgtggagaccttctacgtggacggcgccgccaacagggagaccaagatcggcaaggccggctacgtgaccgacaggggcaggaagaagatcgtgagcctgaccgagaccaccaaccagaagaccgagctgcaggccatctgcatcgccctgcaggacagcggcagcgaggtgaacatcgtgaccgacagccagtacgccctgggcatcatccaggcccagcccgacaagagcgagagcgagctggtgaaccagatcatcgagcagctgatgaagaaggagagggtgtacctgagctgggtgcccgcccacaagggcatcggcggcaacgagcaggtggacaagctggtgagcagcggcatcaggaaggtgctgaagaccctggagcccttcaggaagcagaaccccggcatcgtgatctaccagtacatggacgacctgtacgtgggcagcgacctggagatcggccagcacaggaccaagtaaagatctctcgaggagctcaagcgggcggatcccccgggctgcaggaattcgatcgcgccaaatttaaatgatcctgatcctttttctgggtaagtaatacgtcaaggagaaaacgaaacgatctgtagttagcggccgcctaattaactaatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcaggggaattc

SEQ ID NO: 6 - DNA, 8682 bp, artificial
  nucleotide sequence of the 96zm651-GPN plasmid vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagccaccatggccgccagagcctctatcctgagaggcggcaagctggacaagtgggagaagatccggctgcggcctggcggcaagaaacggtacatgatcaagcacctcgtgtgggccagccgggagctggaaagattcgccctgaaccccggactgctggaaaccagcgagggctgcaagcagatcatgaagcagctgcagcctgccctgcagaccggcaccgaggaactgcggagcctgtacaacaccgtggccaccctgtactgtgtgcacgagggcgtggaagtgcgggacaccaaagaggccctggaccggatcgaggaagaacagaacaagatccagcagaagattcagcagaaaacccagcaggccgccgatggcaaggtgtcccagaactaccccatcgtgcagaacctgcagggccagatggtgcaccagaagctgagccccagaaccctgaacgcctgggtgaaagtgatcgaggaaaaggccttcagccccgaagtgatccccatgttcaccgccctgagcgaaggcgccaccccccaggacctgaacaccatgctgaacaccgtgggaggacaccaggctgccatgcagatgctgaaggacaccatcaacgaagaggccgccgagtgggacagactgcaccctgtgcacgccggacctatcgcccctggccagatgagagagcccagaggcagcgatatcgccggcaccaccagcaccctgcaggaacagatcgcctggatgaccagcaacccccccatccccgtgggcgacatctacaagcggtggatcatcctgggcctgaacaagatcgtgcggatgtacagccccgtgtccatcctggacatcaagcagggccccaaagagcccttccgggactacgtggaccggttcttcaagaccctgcgggccgagcaggccacccaggaagtgaagaactggatgaccgacaccctgctggtgcagaacgccaaccccgactgcaagaccatcctgaaggccctgggacctggcgccaccctggaagagatgatgaccgcctgtcagggcgtgggcggaccttctcacaaggccagagtgctggccgaggccatgagccagaccaacagcgtgaacatcctgatgcagaagtccaacttcaagggcaacaagcggatggtcaagtgcttcaactgcggcaaagagggccacattgcccggaactgcagagcccccagaaagaaaggctgctggaagtgtgggaaagaggggcaccagatgaaggactgcaccgagcggcaggccaacttcctgggcaaaatctggcctagccacaagggcagacccggcaacttcctgcagaatagacccgagcctacagcccctcccgccgagagcttcagattcgaggaaaccacccctgcccccaagcaggaaagcaaggacagagaggccctgacctccctgaagtccctgttcggcagcgaccccctgagccagtttttcagagagaacctcgcctttccacagggcaaggcccgggagtttcccagcgaacaggccagagccaactcccccaccagcagagaactgcaggtccggggcgacaatcctagaagcgaggccggcgtggagagacagggcagcctgaacttccctcagatcaccctgtggcagaggcccctggtgtctatcaaagtgggcggccagatcaaagaagccctgctgaataccggcgctggcgacaccgtgctggaagaaatcaacctgcccggcaagtggaagcccaagatgatcggcggcatcggcggattcatcgaagtgcggcagtacgaccagatccccatggaaatctgcggcaagaaggccatcggcaccgtcctcgtgggccccacccccgtgaacatcatcggccggaacatgctgacacagctgggctgcaccctgaactttcccatcagccccatcgagacagtgcccgtgaagctgaagcccggcatggacggccccaaagtgaagcagtggcctctcaccgaggaaaagatcaaggccctgacagccatctgcgaggaaatggaaaaagagggcaagatcaccaagatcggccccgagaacccctacaacacccccgtgttcgccatcaagaagaaagacagcaccaagtggcggaagctggtggattttcgggagctgaacaagcggacccaggacttctgggaggtgcagctgggcatccctcaccctgccggcctgaagaaaaagaaaagcgtgacagtgctggacgtgggcgacgcctacttcagcgtgcccctggacgagagcttccggaagtacaccgccttcaccatccccagcaccaacaacgagacacccggcatcagataccagtacaacgtgctgccccagggctggaaaggcagccccgccatcttccagagcagcatgaccctggaaggcctgatctacagcaagaagcggcaggaaatcctggacctctgggtgtaccacacccagggctttttccctgactggcaaaactacacccccggacccggcgtgagataccccctgaccttcggctggtgcttcaagctggtgcccgtggaccctggcgaggtggaagaggccaacgagggcgagaacaactgcctgctgcaccctatgtcccagcagggcatggacgacgaccaccgggaggtgctgaagtggaagttcgacagccacctggcccacaagcacatggccagagaactgcaccccgagtactacaaggactgcatgggcggcaagtggtccaagagcagcatcgtgggctggcctgccgtgcgggagcggatcagaagaaccgagcctgccgccgaaggcgtgggagccgccagccaggacctggataagtacggcgccctgaccagcagcaacacctccaccaccaacgccgcctgtgcctggctggaagcccaggaagaggaagaagtcggcttccccgtcagacctcaggtgccactgcggcccatgacatacaaggccgccgtggacctgtcattcttcctgaaagagaagggcggcctggaagggctgcgggagcatctgctgaaatggggcttcaccacccccgacaagaagcaccagaaagaaccccccttcctgtggatgggctacgagctgcatcccgacaagtggaccgtgcagcccatccagctggccgagaaggacagctggaccgtgaacgacatccagaaactcgtgggcaagctgaactgggccagccagatctacgccggcatcaaagtgcgccagctgtgcaagctgctgagaggcgccaaagccctgaccgacatcgtgcctctgacagaggaagccgagctggaactggccgaaaacaaagagatcctcaaagaacccgtccacggcgtgtactacgaccccagcaaggacctgatcgccgagatccagaagcagggccacgaccagtggacctaccagatctaccaggaacccttcaagaacctcaagaccggcaagtacgccaagatgcggaccgcccacaccaacgacgtgaaacagctgaccgaggccgtgcagaagatcgccctggaatccatcgtgatctggggaaagatccccaagttccggctgcccatccagaaagagacatgggaaacctggtggaccgactactggcaggccacctggattcccgagtgggagttcgtgaacacccccctgctggtcaagctgtggtatcagctcgagaaagaacctatcgtgggcgccgagacattctatgtggacggcgctgccaacagagagacaaagctgggcaaggccggctacatcaccgaccggggcagacagaaaatcgtgaccctgaccgaaaccaccaaccagaaaacagagctgcaggccatctacctggccctgcaggatagcggcagcgaagtgaacatcgtgaccgacagccagtacgccctgggcatcatccaggcccaccccgacaagtctgagagcgagctggtcaaccagattatcgagcagctgatcaagaaagaacgggtgtacctgagctgggtgcccgcccacaagggaatcggcggcaacgaacaggtggacaagctggtgtccaagggcatccggaaagtgctgaagatcctggaacccttccgggcccagaaccccgatatcgtgatctaccagtacatggacgacctgtacgtgggcagcgacctggaaatcggccagcaccgggccaagtgatgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctc

SEQ ID NO: 7 - DNA, 8682 bp, artificial
  nucleotide sequence of the 96ZM651-GPN plasmid vector bottom strand
agcgcgcaaagccactactgccacttttggagactgtgtacgtcgagggcctctgccagtgtcgaacagacattcgcctacggccctcgtctgttcgggcagtcccgcgcagtcgcccacaaccgcccacagccccgaccgaattgatacgccgtagtctcgtctaacatgactctcacgtggtatacgccacactttatggcgtgtctacgcattcctcttttatggcgtagtctaaccgataaccggtaacgtatgcaacataggtatagtattatacatgtaaatataaccgagtacaggttgtaatggcggtacaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctagcggacctctgcggtaggtgcgacaaaactggaggtatcttctgtggccctggctaggtcggaggtagccgagcgtagagaggaagtgcgcgggcggcgggatggactccggcggtaggtgcggccaactcagcgcaagacggcggagggcggacaccacggaggacttgacgcaggcggcagatccattcaaatttcgagtccagctctggcccggaaacaggccgcgagggaacctcggatggatctgagtcggccgagaggtgcgaaacggactgggacgaacgagttgagatcaattgccacctcccgtcacatcagactcgtcatgagcaacgacggcgcgcgcggtggtctgtattatcgactgtctgattgtctgacaaggaaaggtacccagaaaagacgtcagtggcagcagctgtgcacactagtctatagcgccggcgagatctcggtggtaccggcggtctcggagataggactctccgccgttcgacctgttcaccctcttctaggccgacgccggaccgccgttctttgccatgtactagttcgtggagcacacccggtcggccctcgacctttctaagcgggacttggggcctgacgacctttggtcgctcccgacgttcgtctagtacttcgtcgacgtcggacgggacgtctggccgtggctccttgacgcctcggacatgttgtggcaccggtgggacatgacacacgtgctcccgcaccttcacgccctgtggtttctccgggacctggcctagctccttcttgtcttgttctaggtcgtcttctaagtcgtcttttgggtcgtccggcggctaccgttccacagggtcttgatggggtagcacgtcttggacgtcccggtctaccacgtggtcttcgactcggggtcttgggacttgcggacccactttcactagctccttttccggaagtcggggcttcactaggggtacaagtggcgggactcgcttccgcggtggggggtcctggacttgtggtacgacttgtggcaccctcctgtggtccgacggtacgtctacgacttcctgtggtagttgcttctccggcggctcaccctgtctgacgtgggacacgtgcggcctggatagcggggaccggtctactctctcgggtctccgtcgctatagcggccgtggtggtcgtgggacgtccttgtctagcggacctactggtcgttgggggggtaggggcacccgctgtagatgttcgccacctagtaggacccggacttgttctagcacgcctacatgtcggggcacaggtaggacctgtagttcgtcccggggtttctcgggaaggccctgatgcacctggccaagaagttctgggacgcccggctcgtccggtgggtccttcacttcttgacctactggctgtgggacgaccacgtcttgcggttggggctgacgttctggtaggacttccgggaccctggaccgcggtgggaccttctctactactggcggacagtcccgcacccgcctggaagagtgttccggtctcacgaccggctccggtactcggtctggttgtcgcacttgtaggactacgtcttcaggttgaagttcccgttgttcgcctaccagttcacgaagttgacgccgtttctcccggtgtaacgggccttgacgtctcgggggtctttctttccgacgaccttcacaccctttctccccgtggtctacttcctgacgtggctcgccgtccggttgaaggacccgttttagaccggatcggtgttcccgtctgggccgttgaaggacgtcttatctgggctcggatgtcggggagggcggctctcgaagtctaagctcctttggtggggacgggggttcgtcctttcgttcctgtctctccgggactggagggacttcagggacaagccgtcgctgggggactcggtcaaaaagtctctcttggagcggaaaggtgtcccgttccgggccctcaaagggtcgcttgtccggtctcggttgagggggtggtcgtctcttgacgtccaggccccgctgttaggatcttcgctccggccgcacctctctgtcccgtcggacttgaagggagtctagtgggacaccgtctccggggaccacagatagtttcacccgccggtctagtttcttcgggacgacttatggccgcgaccgctgtggcacgaccttctttagttggacgggccgttcaccttcgggttctactagccgccgtagccgcctaagtagcttcacgccgtcatgctggtctaggggtacctttagacgccgttcttccggtagccgtggcaggagcacccggggtgggggcacttgtagtagccggccttgtacgactgtgtcgacccgacgtgggacttgaaagggtagtcggggtagctctgtcacgggcacttcgacttcgggccgtacctgccggggtttcacttcgtcaccggagagtggctccttttctagttccgggactgtcggtagacgctcctttacctttttctcccgttctagtggttctagccggggctcttggggatgttgtgggggcacaagcggtagttcttctttctgtcgtggttcaccgccttcgaccacctaaaagccctcgacttgttcgcctgggtcctgaagaccctccacgtcgacccgtagggagtgggacggccggacttctttttcttttcgcactgtcacgacctgcacccgctgcggatgaagtcgcacggggacctgctctcgaaggccttcatgtggcggaagtggtaggggtcgtggttgttgctctgtgggccgtagtctatggtcatgttgcacgacggggtcccgacctttccgtcggggcggtagaaggtctcgtcgtactgggaccttccggactagatgtcgttcttcgccgtcctttaggacctggagacccacatggtgtgggtcccgaaaaagggactgaccgttttgatgtgggggcctgggccgcactctatgggggactggaagccgaccacgaagttcgaccacgggcacctgggaccgctccaccttctccggttgctcccgctcttgttgacggacgacgtgggatacagggtcgtcccgtacctgctgctggtggccctccacgacttcaccttcaagctgtcggtggaccgggtgttcgtgtaccggtctcttgacgtggggctcatgatgttcctgacgtacccgccgttcaccaggttctcgtcgtagcacccgaccggacggcacgccctcgcctagtcttcttggctcggacggcggcttccgcaccctcggcggtcggtcctggacctattcatgccgcgggactggtcgtcgttgtggaggtggtggttgcggcggacacggaccgaccttcgggtccttctccttcttcagccgaaggggcagtctggagtccacggtgacgccgggtactgtatgttccggcggcacctggacagtaagaaggactttctcttcccgccggaccttcccgacgccctcgtagacgactttaccccgaagtggtgggggctgttcttcgtggtctttcttggggggaaggacacctacccgatgctcgacgtagggctgttcacctggcacgtcgggtaggtcgaccggctcttcctgtcgacctggcacttgctgtaggtctttgagcacccgttcgacttgacccggtcggtctagatgcggccgtagtttcacgcggtcgacacgttcgacgactctccgcggtttcgggactggctgtagcacggagactgtctccttcggctcgaccttgaccggcttttgtttctctaggagtttcttgggcaggtgccgcacatgatgctggggtcgttcctggactagcggctctaggtcttcgtcccggtgctggtcacctggatggtctagatggtccttgggaagttcttggagttctggccgttcatgcggttctacgcctggcgggtgtggttgctgcactttgtcgactggctccggcacgtcttctagcgggaccttaggtagcactagacccctttctaggggttcaaggccgacgggtaggtctttctctgtaccctttggaccacctggctgatgaccgtccggtggacctaagggctcaccctcaagcacttgtggggggacgaccagttcgacaccatagtcgagctctttcttggatagcacccgcggctctgtaagatacacctgccgcgacggttgtctctctgtttcgacccgttccggccgatgtagtggctggccccgtctgtcttttagcactgggactggctttggtggttggtcttttgtctcgacgtccggtagatggaccgggacgtcctatcgccgtcgcttcacttgtagcactggctgtcggtcatgcgggacccgtagtaggtccgggtggggctgttcagactctcgctcgaccagttggtctaatagctcgtcgactagttctttcttgcccacatggactcgacccacgggcgggtgttcccttagccgccgttgcttgtccacctgttcgaccacaggttcccgtaggcctttcacgacttctaggaccttgggaaggcccgggtcttggggctatagcactagatggtcatgtacctgctggacatgcacccgtcgctggacctttagccggtcgtggcccggttcactactcctaggtctagacgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcgtccgtacgacccctacgccacccgagatacccatgggtccacgacttcttaactgggccaaggaggacccggtctttcttcgtccgtgtaggggaagagacactgtgtgggacaggtgcggggaccaagaatcaaggtcggggtgagtatcctgtgagtatcgagtcctcccgaggcggaagttagggtgggcgatttcatgaacctcgccagagagggagggagtagtcgggtggtttggtttggatcggaggttctcacccttctttaatttcgttctatccgataattcacgtctccctctcttttacggaggttgtacactccttcattactctctttagtatcttaaaattccggtactaaattccggtagtaccggaattagaaggcgaaggagcgagtgactgagcgacgcgagccagcaagccgacgccgctcgccatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcttgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggactgagccccccccccccgcgactccagacggagcacttcttccacaacgactgagtatggtccggacttagcggggtagtaggtcggtctttcactccctcggtgccaactactctcgaaacaacatccacctggtcaaccactaaaacttgaaaacgaaacggtgccttgccagacgcaacagcccttctacgcactagactaggaagttgagtcgttttcaagctaaataagttgtttcggcggcagggcagttcagtcgcattacgagacggtcacaatgttggttaattggttaagactaatctttttgagtagctcgtagtttactttgacgttaaataagtatagtcctaatagttatggtataaaaactttttcggcaaagacattacttcctcttttgagtggctccgtcaaggtatcctaccgttctaggaccatagccagacgctaaggctgagcaggttgtagttatgttggataattaaaggggagcagtttttattccaatagttcactctttagtggtactcactgctgacttaggccactcttaccgttttcgaatacgtaaagaaaggtctgaacaagttgtccggtcggtaatgcgagcagtagttttagtgagcgtagttggtttggcaataagtaagcactaacgcggactcgctctgctttatgcgctagcgacaattttcctgttaatgtttgtccttagcttacgttggccgcgtccttgtgacggtcgcgtagttgttataaaagtggacttagtcctataagaagattatggaccttacgacaaaagggcccctagcgtcaccactcattggtacgtagtagtcctcatgcctattttacgaactaccagccttctccgtatttaaggcagtcggtcaaatcagactggtagagtagacattgtagtaaccgttgcgatggaaacggtacaaagtctttgttgagaccgcgtagcccgaagggtatgttagctatctaacagcgtggactaacgggctgtaatagcgctcgggtaaatatgggtatatttagtcgtaggtacaaccttaaattagcgccggagctcgttctgcaaagggcaacttataccgagtattgtggggaacataatgacaaatacattcgtctgtcaaaataacaagtactactatataaaaatagaacacgttacattgtagtctctaaaactctgtgttgcaccgaaaggggggggggggtaataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagagcgcgcaaagccactactgccacttttggag

SEQ ID NO: 8 - protein, 1411 aa, artificial
  encoded GagPolNef (GPN) amino acid sequence of the 96ZM651-GPN plasmid vector
MAARASILRGGKLDKWEKIRLRPGGKKRYMIKHLVWASRELERFALNPGLLETSEGCKQIMKQLQPALQTGTEELRSLYNTVATLYCVHEGVEVRDTKEALDRIEEEQNKIQQKIQQKTQQAADGKVSQNYPIVQNLQGQMVHQKLSPRTLNAWVKVIEEKAFSPEVIPMFTALSEGATPQDLNTMLNTVGGHQAAMQMLKDTINEEAAEWDRLHPVHAGPIAPGQMREPRGSDIAGTTSTLQEQIAWMTSNPPIPVGDIYKRWIILGLNKIVRMYSPVSILDIKQGPKEPFRDYVDRFFKTLRAEQATQEVKNWMTDTLLVQNANPDCKTILKALGPGATLEEMMTACQGVGGPSHKARVLAEAMSQTNSVNILMQKSNFKGNKRMVKCFNCGKEGHIARNCRAPRKKGCWKCGKEGHQMKDCTERQANFLGKIWPSHKGRPGNFLQNRPEPTAPPAESFRFEETTPAPKQESKDREALTSLKSLFGSDPLSQFFRENLAFPQGKAREFPSEQARANSPTSRELQVRGDNPRSEAGVERQGSLNFPQITLWQRPLVSIKVGGQIKEALLNTGAGDTVLEEINLPGKWKPKMIGGIGGFIEVRQYDQIPMEICGKKAIGTVLVGPTPVNIIGRNMLTQLGCTLNFPISPIETVPVKLKPGMDGPKVKQWPLTEEKIKALTAICEEMEKEGKITKIGPENPYNTPVFAIKKKDSTKWRKLVDFRELNKRTQDFWEVQLGIPHPAGLKKKKSVTVLDVGDAYFSVPLDESFRKYTAFTIPSTNNETPGIRYQYNVLPQGWKGSPAIFQSSMTLEGLIYSKKRQEILDLWVYHTQGFFPDWQNYTPGPGVRYPLTFGWCFKLVPVDPGEVEEANEGENNCLLHPMSQQGMDDDHREVLKWKFDSHLAHKHMARELHPEYYKDCMGGKWSKSSIVGWPAVRERIRRTEPAAEGVGAASQDLDKYGALTSSNTSTTNAACAWLEAQEEEEVGFPVRPQVPLRPMTYKAAVDLSFFLKEKGGLEGLREHLLKWGFTTPDKKHQKEPPFLWMGYELHPDKWTVQPIQLAEKDSWTVNDIQKLVGKLNWASQIYAGIKVRQLCKLLRGAKALTDIVPLTEEAELELAENKEILKEPVHGVYYDPSKDLIAEIQKQGHDQWTYQIYQEPFKNLKTGKYAKMRTAHTNDVKQLTEAVQKIALESIVIWGKIPKFRLPIQKETWETWWTDYWQATWIPEWEFVNTPLLVKLWYQLEKEPIVGAETFYVDGAANRETKLGKAGYITDRGRQKIVTLTETTNQKTELQAIYLALQDSGSEVNIVTDSQYALGIIQAHPDKSESELVNQIIEQLIKKERVYLSWVPAHKGIGGNEQVDKLVSKGIRKVLKILEPFRAQNPDIVIYQYMDDLYVGSDLEIGQHRAK

SEQ ID NO: 9 - protein, 1411 aa, artificial; identical to SEQ ID NO: 8
  HIV - 96ZM651-GPN

SEQ ID NO: 10 - DNA, 6373 bp, artificial
  nucleotide sequence in plasmid map of 97CN54-gp140, top strand
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagccaccatggaccgggccaagctgctgctcctgctcctgctgctgctcctgcctcaggcccaggctgtgggcaatctgtgggtgaccgtgtactacggcgtgccagtgtggaagggcgccacaaccaccctgttttgcgccagcgacgccaaggcctacgacaccgaggtgcacaacgtgtgggccacccatgcctgtgtgcccgccgaccccaaccctcaggaaatggtcctggaaaacgtgaccgagaacttcaacatgtggaagaacgagatggtcaaccagatgcaggaagatgtcatcagcctgtgggaccagagcctgaagccctgcgtgaagctgacccccctgtgcgtgaccctggaatgccggaacgtgtccagcaacagcaacgacacctaccacgagacataccacgagagcatgaaggaaatgaagaactgcagcttcaacgccaccaccgtcgtgcgggaccggaagcagaccgtgtacgccctgttctaccggctggacatcgtgcccctgaccaagaagaactacagcgagaacagcagcgagtactaccggctgatcaactgcaacaccagcgccatcacccaggcctgccccaaagtgaccttcgaccccatccccatccactactgcacccctgctggctacgccatcctgaagtgcaacgacaagatcttcaacggcaccggcccctgccacaatgtgtccaccgtgcagtgcacccacggcatcaagcccgtggtgtccacccagctgctgctgaatggcagcctggccgagggcgagatcatcatcagaagcgagaacctgaccaacaacgtcaagaccatcatcgtgcacctgaaccagagcgtggagatcgtgtgcaccagacccggcaacaacacccggaagtccatcagaatcggccctggccagaccttttacgccaccggcgacatcatcggcgatatccggcaggcccactgcaacatctccgaggacaagtggaacgagacactgcagcgggtgtccaagaagctggccgagcacttccagaacaagacaatcaagttcgccagcagcagcggaggcgacctggaagtgaccacccacagcttcaactgcagaggcgagttcttctactgcaatacctccggcctgttcaacggcgcctacacccccaacggcaccaagagcaacagcagcagcatcatcaccatcccttgccggatcaagcagatcatcaatatgtggcaggaagtgggcagagccatgtacgcccctcccatcaagggcaacatcacatgcaagtccaacatcaccggcctgctgctcgtcagagatggcggcaccgagcccaacgacaccgaaaccttcagacccggcggaggcgatatgcggaacaactggcggagcgagctgtacaagtacaaggtggtggagatcaagcctctgggcgtggcccctaccaccaccaagcggagagtggtggagagggaaaaggccgccgtgggcatcggagccgtgtttctgggcttcctgggagtggccggctctacaatgggagccgccagcatcacactgaccgtgcaggccagacagctgctcagcggcatcgtgcagcagcagagcaacctgctgagagccatcgaggcccagcagcatctgctgcagctgaccgtctggggcattaagcagctccagacaagagtcctggctatcgagagatacctgaaggaccagcagctcctggggatttggggctgctctggaaaactcatctgcactactgctgtgccctggaatagcagctggtccaacaagagccagaaagaaatctgggacaacatgacctggatgcagtgggataaagaaattagtaattacacaaacacagtgtaccggctgctggaagagagccagaaccagcaggaacggaacgagaaggacctgctggctctggacagctgatgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 6373 bp, artificial
  nucleotide sequence in plasmid map of 97CN54-gp140, bottom strand
agcgcgcaaagccactactgccacttttggagactgtgtacgtcgagggcctctgccagtgtcgaacagacattcgcctacggccctcgtctgttcgggcagtcccgcgcagtcgcccacaaccgcccacagccccgaccgaattgatacgccgtagtctcgtctaacatgactctcacgtggtatacgccacactttatggcgtgtctacgcattcctcttttatggcgtagtctaaccgataaccggtaacgtatgcaacataggtatagtattatacatgtaaatataaccgagtacaggttgtaatggcggtacaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctagcggacctctgcggtaggtgcgacaaaactggaggtatcttctgtggccctggctaggtcggaggtagccgagcgtagagaggaagtgcgcgggcggcgggatggactccggcggtaggtgcggccaactcagcgcaagacggcggagggcggacaccacggaggacttgacgcaggcggcagatccattcaaatttcgagtccagctctggcccggaaacaggccgcgagggaacctcggatggatctgagtcggccgagaggtgcgaaacggactgggacgaacgagttgagatcaattgccacctcccgtcacatcagactcgtcatgagcaacgacggcgcgcgcggtggtctgtattatcgactgtctgattgtctgacaaggaaaggtacccagaaaagacgtcagtggcagcagctgtgcacactagtctatagcgccggcgagatctcggtggtacctggcccggttcgacgacgaggacgaggacgacgacgaggacggagtccgggtccgacacccgttagacacccactggcacatgatgccgcacggtcacaccttcccgcggtgttggtgggacaaaacgcggtcgctgcggttccggatgctgtggctccacgtgttgcacacccggtgggtacggacacacgggcggctggggttgggagtcctttaccaggaccttttgcactggctcttgaagttgtacaccttcttgctctaccagttggtctacgtccttctacagtagtcggacaccctggtctcggacttcgggacgcacttcgactggggggacacgcactgggaccttacggccttgcacaggtcgttgtcgttgctgtggatggtgctctgtatggtgctctcgtacttcctttacttcttgacgtcgaagttgcggtggtggcagcacgccctggccttcgtctggcacatgcgggacaagatggccgacctgtagcacggggactggttcttcttgatgtcgctcttgtcgtcgctcatgatggccgactagttgacgttgtggtcgcggtagtgggtccggacggggtttcactggaagctggggtaggggtaggtgatgacgtggggacgaccgatgcggtaggacttcacgttgctgttctagaagttgccgtggccggggacggtgttacacaggtggcacgtcacgtgggtgccgtagttcgggcaccacaggtgggtcgacgacgacttaccgtcggaccggctcccgctctagtagtagtcttcgctcttggactggttgttgcagttctggtagtagcacgtggacttggtctcgcacctctagcacacgtggtctgggccgttgttgtgggccttcaggtagtcttagccgggaccggtctggaaaatgcggtggccgctgtagtagccgctataggccgtccgggtgacgttgtagaggctcctgttcaccttgctctgtgacgtcgcccacaggttcttcgaccggctcgtgaaggtcttgttctgttagttcaagcggtcgtcgtcgcctccgctggaccttcactggtgggtgtcgaagttgacgtctccgctcaagaagatgacgttatggaggccggacaagttgccgcggatgtgggggttgccgtggttctcgttgtcgtcgtcgtagtagtggtagggaacggcctagttcgtctagtagttatacaccgtccttcacccgtctcggtacatgcggggagggtagttcccgttgtagtgtacgttcaggttgtagtggccggacgacgagcagtctctaccgccgtggctcgggttgctgtggctttggaagtctgggccgcctccgctatacgccttgttgaccgcctcgctcgacatgttcatgttccaccacctctagttcggagacccgcaccggggatggtggtggttcgcctctcaccacctctcccttttccggcggcacccgtagcctcggcacaaagacccgaaggaccctcaccggccgagatgttaccctcggcggtcgtagtgtgactggcacgtccggtctgtcgacgagtcgccgtagcacgtcgtcgtctcgttggacgactctcggtagctccgggtcgtcgtagacgacgtcgactggcagaccccgtaattcgtcgaggtctgttctcaggaccgatagctctctatggacttcctggtcgtcgaggacccctaaaccccgacgagaccttttgagtagacgtgatgacgacacgggaccttatcgtcgaccaggttgttctcggtctttctttagaccctgttgtactggacctacgtcaccctatttctttaatcattaatgtgtttgtgtcacatggccgacgaccttctctcggtcttggtcgtccttgccttgctcttcctggacgaccgagacctgtcgactactcctaggtctagacgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcgtccgtacgacccctacgccacccgagatacccatgggtccacgacttcttaactgggccaaggaggacccggtctttcttcgtccgtgtaggggaagagacactgtgtgggacaggtgcggggaccaagaatcaaggtcggggtgagtatcctgtgagtatcgagtcctcccgaggcggaagttagggtgggcgatttcatgaacctcgccagagagggagggagtagtcgggtggtttggtttggatcggaggttctcacccttctttaatttcgttctatccgataattcacgtctccctctcttttacggaggttgtacactccttcattactctctttagtatcttaaaattccggtactaaattccggtagtaccggaattagaaggcgaaggagcgagtgactgagcgacgcgagccagcaagccgacgccgctcgccatagtcgagtgagtttccgccattatgccaataggtgtcttagtcccctattgcgtcctttcttgtacactcgttttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcttgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttaatttttacttcaaaatttagttagatttcatatatactcatttgaaccagactgtcaatggttacgaattagtcactccgtggatagagtcgctagacagataaagcaagtaggtatcaacggactgagccccccccccccgcgactccagacggagcacttcttccacaacgactgagtatggtccggacttagcggggtagtaggtcggtctttcactccctcggtgccaactactctcgaaacaacatccacctggtcaaccactaaaacttgaaaacgaaacggtgccttgccagacgcaacagcccttctacgcactagactaggaagttgagtcgttttcaagctaaataagttgtttcggcggcagggcagttcagtcgcattacgagacggtcacaatgttggttaattggttaagactaatctttttgagtagctcgtagtttactttgacgttaaataagtatagtcctaatagttatggtataaaaactttttcggcaaagacattacttcctcttttgagtggctccgtcaaggtatcctaccgttctaggaccatagccagacgctaaggctgagcaggttgtagttatgttggataattaaaggggagcagtttttattccaatagttcactctttagtggtactcactgctgacttaggccactcttaccgttttcgaatacgtaaagaaaggtctgaacaagttgtccggtcggtaatgcgagcagtagttttagtgagcgtagttggtttggcaataagtaagcactaacgcggactcgctctgctttatgcgctagcgacaattttcctgttaatgtttgtccttagcttacgttggccgcgtccttgtgacggtcgcgtagttgttataaaagtggacttagtcctataagaagattatggaccttacgacaaaagggcccctagcgtcaccactcattggtacgtagtagtcctcatgcctattttacgaactaccagccttctccgtatttaaggcagtcggtcaaatcagactggtagagtagacattgtagtaaccgttgcgatggaaacggtacaaagtctttgttgagaccgcgtagcccgaagggtatgttagctatctaacagcgtggactaacgggctgtaatagcgctcgggtaaatatgggtatatttagtcgtaggtacaaccttaaattagcgccggagctcgttctgcaaagggcaacttataccgagtattgtggggaacataatgacaaatacattcgtctgtcaaaataacaagtactactatataaaaatagaacacgttacattgtagtctctaaaactctgtgttgcaccgaaaggggggggggggtaataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcag

SEQ ID NO: 12 - protein, 652 aa, artificial
  encoded gp140 amino acid sequence in plasmid map of 97CN54-gp140
MDRAKLLLLLLLLLLPQAQAVGNLWVTVYYGVPVWKGATTTLFCASDAKAYDTEVHNVWATHACVPADPNPQEMVLENVTENFNMWKNEMVNQMQEDVISLWDQSLKPCVKLTPLCVTLECRNVSSNSNDTYHETYHESMKEMKNCSFNATTVVRDRKQTVYALFYRLDIVPLTKKNYSENSSEYYRLINCNTSAITQACPKVTFDPIPIHYCTPAGYAILKCNDKIFNGTGPCHNVSTVQCTHGIKPVVSTQLLLNGSLAEGEIIIRSENLTNNVKTIIVHLNQSVEIVCTRPGNNTRKSIRIGPGQTFYATGDIIGDIRQAHCNISEDKWNETLQRVSKKLAEHFQNKTIKFASSSGGDLEVTTHSFNCRGEFFYCNTSGLFNGAYTPNGTKSNSSSIITIPCRIKQIINMWQEVGRAMYAPPIKGNITCKSNITGLLLVRDGGTEPNDTETFRPGGGDMRNNWRSELYKYKVVEIKPLGVAPTTTKRRVVEREKAAVGIGAVFLGFLGVAGSTMGAASITLTVQARQLLSGIVQQQSNLLRAIEAQQHLLQLTVWGIKQLQTRVLAIERYLKDQQLLGIWGCSGKLICTTAVPWNSSWSNKSQKEIWDNMTWMQWDKEISNYTNTVYRLLEESQNQQERNEKDLLALDS